(19) United States
(12) Patent Application Publication
(10) Pub. No.: US 2025/0011388 A1
(43) Pub. Date: Jan. 9, 2025

(54) VARIANT CTLA4 PROTEINS

(71) Applicants: MONASH UNIVERSITY, Clayton (AU); THE WALTER AND ELIZA HALL INSTITUTE OF MEDICAL RESEARCH, 3052 (AU)

(72) Inventors: Ross Alexander Dickins, Clayton (AU); Ethan Paul Oxley, Clayton (AU); Katharine Jennifer Goodall, Clayton (AU); Maximilian Meredith Garwood, Clayton (AU); Nadia Jean Kershaw, Parkville (AU)

(21) Appl. No.: 18/705,274

(22) PCT Filed: Oct. 28, 2022
(86) PCT No.: PCT/AU2022/051296

§ 371 Date: Apr. 26, 2024

(30) Foreign Application Priority Data
Oct. 28, 2021 (AU) 2021903455

(51) Int. Cl. C07K 14/705; A61K 38/00; A61P 35/00

(52) U.S. Cl. CPC C07K 14/70521; A61P 35/00; A61K 38/00; C07K 2319/30

(57) ABSTRACT

The present application relates to CTLA4 fusion proteins, compositions comprising the same, and uses thereof, wherein the CTLA4 fusion proteins do not displace cis-bound PDL1 from B7 upon binding.

### FIELD OF THE INVENTION

The present application relates to CTLA4 fusion proteins, compositions comprising the same, and uses thereof.

### RELATED APPLICATION

The present application claims priority from Australian provisional patent application AU 2021903455, the entire contents of which are hereby incorporated by reference.

### BACKGROUND OF THE INVENTION

T cell activation depends on sustained interaction with APCs expressing surface ligands for the T cell receptor (TCR) and the co-stimulatory receptor CD28. Without co-stimulation, TCR signalling induces T cell unresponsiveness. Upon activation T cells induce CTLA4, a homodimeric transmembrane protein homologous to CD28 but lacking signalling activity.

Cell surface CTLA4 and CD28 share the APC-expressed co-stimulatory ligands CD80 (B7-1) and CD86 (B7-2). Whereas CD28 interacts monovalently with its ligands, CTLA4 binds bivalently with high avidity. Hence surface CTLA4 outcompetes CD28 for their ligands, inhibiting co-stimulation and limiting T cell activation.

CTLA4 therefore inhibits the immune response in two principal ways: it competes with CD28 for the B7 ligands and thus blocks co-stimulation.

Regulatory T cells (Tregs) constitutively express abundant CTLA4, and inhibit co-stimulation by an additional mechanism whereby CD80 and CD86 are depleted from the APC surface through CTLA4-mediated transendocytosis. CTLA4-deficient mice develop fatal T cell-mediated multi-organ autoimmunity and in human studies reduced CTLA4 activity is associated with autoimmune disease, highlighting an essential role for CTLA4 in limiting steady state T cell activity. CTLA4 checkpoint blockade with monoclonal antibodies such as ipilimumab can trigger potent anti-tumour T cell immunity in many cancers, but can also lead to immune-related adverse events including dose-limiting autoimmunity.

Due to the critical role of the B7 (i.e., B7-1 and B7-2) co-stimulatory pathway in promoting and maintaining immune response, therapeutic agents designed to antagonize the pathway are in clinical use for the treatment of autoimmune diseases and disorders. Abatacept (Orencia®) is a CTLA4-Ig fusion protein consisting of the extracellular binding domain of CTLA4 linked to the Fc domain of a human IgG. Abatacept was developed to inhibit B7-1-mediated co-stimulation and is approved for the treatment of rheumatoid arthritis (RA) and in clinical trials for a number of other autoimmune indications. Belatacept was further developed and differs from abatacept by only two amino acids and binds with greater affinity to B7-1 compared to abatacept.

Similarly to membrane-bound CTLA4, CTLA4-Ig-based therapies including abatacept and belatacept, bind APC surface B7-1/B7-2 to block co-stimulation. Existing CTLA4-Ig based therapeutics provide a potent immunosuppressive effect that is often accompanied with wide ranging side effects.

The ability to reduce the side effect profile, and fine-tune pharmacological interventions via the CTLA4-B7 axis is desirable. Accordingly, there is a need for alternative and improved therapies which target the CTLA4/B7 axis.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention is based on the recognition by the inventors, that CTLA4-Ig fusion proteins that do not displace cis-bound PDL1 from B7-1 (CD80) upon binding, or only partly displace cis-bound PDL1 from CD80, are immunostimulatory. This is in contrast to other known CTLA-4-Ig fusion proteins, such as abatacept, which are known to be immunosuppressive.

In a first aspect the present invention provides a fusion protein comprising:
a first portion comprising or consisting of the amino acid sequence of a CTLA4 protein, or functional variant thereof,
a second portion comprising an Fc region of an antibody, wherein the fusion protein is capable of covalent bonding to form a dimer, preferably a homodimer, and of bivalent ligand binding, wherein the first portion comprises one or more mutations, insertions, substitutions or deletions for reducing the stability of the CTLA4 homodimerisation interface compared to the homodimerisation interface of wild-type CTLA4 protein, such that upon bivalent binding of the fusion protein to CD80, PDL1 remains cis-bound to CD80.

Preferably, upon bivalent binding of the fusion protein to CD80, PDL1 remains cis-bound to CD80 such that there is no liberation or substantially no liberation of PDL1 upon binding of the fusion protein to CD80. In alternative embodiments, upon bivalent binding, PDL1 is partially liberated from binding to CD80 such that some PDL1 molecules remain cis-bound to CD80.

In a preferred embodiment, the covalent bonding is disulphide bonding.

The first portion preferably comprises the sequence of a CTLA4 protein (whether derived from the soluble isoform of the protein, or transmembrane isoform), that binds to or specifically binds to one or both of CD80 and CD86.

Preferably the first portion comprises at least the sequence of the ligand binding domain of a CTLA4 protein, wherein the sequence of the ligand binding domain is common to both the soluble and transmembrane forms of the protein. Preferably, the first portion comprises the sequence of the ligand binding domain of a CTLA4 protein, along with residues C-terminal to the ligand binding domain for facilitating homodimerisation of the protein.

An example of the ligand binding domain of a CTLA4 protein is defined herein in SEQ ID NO: 1. The amino acid sequence set forth in SEQ ID NO: 1 corresponds to residues M3 to 1117 of mature CTLA4 protein (numbered according to the convention used for the transmembrane form of the protein). In other words, M1 in SEQ ID NO: 1 and 2, corresponds to M3 of the CTLA4 transmembrane protein numbered according to convention.

In a preferred embodiment, the first portion of the fusion protein comprises or consists the amino acid sequence set forth in SEQ ID NO: 1, or a functional variant thereof having at least 80%, at least 81%, at least 82%, at least 83%, at least 84%, at least 85%, at least 86%, at least 87%, at least 88%, at least 89%, at least 90%, at least 91%, at least 91%, at least 92%, at least 93%, at least 94%, at least 95%, at least 96%, at least 97% at least 98% or at least 99% sequence identity thereto.

Alternatively, the first portion of the fusion protein comprises or consists of the amino acid sequence set forth in SEQ ID NO: 2 or a functional variant thereof having at least 80%, at least 81%, at least 82%, at least 83%, at least 84%, at least 85%, at least 86%, at least 87%, at least 88%, at least 89%, at least 90%, at least 91%, at least 91%, at least 92%, at least 93%, at least 94%, at least 95%, at least 96%, at least 97% at least 98% or at least 99% sequence identity thereto.

Further still, the first portion of the fusion protein may comprise or consist of the amino acid sequence set forth in SEQ ID NO: 3 or a functional variant thereof having at least 80%, at least 81%, at least 82%, at least 83%, at least 84%, at least 85%, at least 86%, at least 87%, at least 88%, at least 89%, at least 90%, at least 91%, at least 91%, at least 92%, at least 93%, at least 94%, at least 95%, at least 96%, at least 97% at least 98% or at least 99% sequence identity thereto. In one example, the functional variant of SEQ ID NO: 3 may comprise an amino acid sequence that is C terminally truncated by at least 1, at least 2, at least 3, at least 4, at least 5, at least 6, at least 7, at least 8, at least 9 or at least 10 amino acid residues, such as the amino acid sequence of SEQ ID NO: 59.

The second portion of the fusion protein preferably comprises the CH2 and CH3 domains of an antibody. In certain embodiments the second portion of the fusion protein also comprises an Fc hinge region (for example, hinge-CH2-CH3).

In a preferred embodiment, there is provided a dimeric protein formed by covalently bonded, preferably disulphide bonded, monomers of the fusion protein described herein.

The present invention contemplates the use of any deletion, insertion, mutation or substitution in the regions of CTLA4 which are known to contribute to homodimerisation, for reducing the stability of the homodimerisation interface of the protein, when dimerised.

Two regions of the transmembrane isoform of CTLA4 are known to contribute to its homodimerisation following formation of a disulphide bond between monomers of the protein via C120:
(1) the ligand-binding domain, defined as the region shared by both the transmembrane and soluble isoforms of CTLA4 and corresponding to the sequence defined herein in SEQ ID NO: 1;
(2) residues between the ligand-binding domain (eg the sequence motif “DPEP”, as defined in SEQ ID NO: 2, residues 116 to 119) and C120.

As such, residues involved in the formation of hydrophobic interactions to form the homodimerisation interface of a CTLA4 protein are generally understood to include amino acid residues V8, V9, L10, A11, S12, S13, and amino acid residues T110, Q111, 1112, Y113, V114, 1115, D116, P117, E118, and P119, or residues at positions equivalent thereto, in the CTLA4 protein.

In the wild-type transmembrane form of CTLA4, residues V8 to S13 interact with residues T110 to D116 following formation of a disulphide bond between the cysteine residues at C120 of the CTLA4 protein (numbering defined according to SEQ ID NO: 2). More specifically, residues V8, L10, Y113 and I115 form a hydrophobic interface with equivalent residues on the adjacent molecule, which contributes to the homodimer interface, along with the “DPEP” motif (residues 116 to 119) and disulphide bonding between cysteine residues at position 120. The amino acid sequence of these regions is depicted in Table 1 herein in the context of SEQ ID NO: 2, and comprising the sequences VVLASS and TQIYVI[DPEP].

Destabilisation of the homodimerisation interface of CTLA4 may be achieved by any means, as further described herein. Destabilisation of the homodimerisation interface may be achieved by insertion, mutation, substitution or deletion of the regions: VVLASS and/or TQIYVI[DPEP] as defined above.

Preferably, destabilisation of the homodimerisation interface is accomplished through the introduction of one or more amino acid insertions or flexible regions, N-terminal to the residues involved in covalent bond, preferably disulphide bond, formation between two monomers of the fusion protein. In any embodiment, the insertion or flexible region is comprised of amino acid residues selected from glycine, serine, alanine residues. The insertion may be a single amino acid residue, or 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid residues.

In further embodiments, destabilisation of the homodimerisation interface of CTLA4 is achieved by substituting or deleting one or more residues N-terminal to the residues of the fusion protein involved in covalent bond, preferably disulphide bond, formation, wherein preferably disulphide bond formation occurs between cysteine residues in the C-terminal 25 amino acids of the first portion, or between cysteine residues in the N-terminal region of the second portion (eg the Fc hinge region). Examples of suitable amino acid residues for substitution include glycine, serine, alanine, lysine and glutamic acid, preferably glycine or serine.

In accordance with the present invention, destabilisation of the homodimerisation interface of the first portion is preferably accomplished through the insertion of one or more residues or a flexible domain, or by introduction of one or more mutations, deletions of substitutions in the region of the first portion corresponding to the homodimeration interface of CTLA4, preferably corresponding to regions of CTLA4 involved in the formation of hydrophobic interactions in the homodimerisation interface.

Accordingly, in any embodiment of the invention, the fusion protein comprises one or more deletions, mutations or substitutions of any one of residues V8, V9, L10, A11, S12, S13, T110, Q111, 1112, Y113, V114, 1115 and D116, or at positions equivalent thereto, in the CTLA4 ligand binding domain (as numbered according to SEQ ID NO: 1, 2 or 3).

Alternatively, the fusion protein may comprise an insertion of one or more amino acid residues or a flexible region in the sequence between the ligand-binding domain and the remainder of the ectodomain of the CTLA4 protein (ie, an insertion in any position between residues I115 and C120, preferably between I115 and P119, or between I115 and E118, or I115 and P117, most preferably between I115 and D116).

In further embodiments, the residues involved in homodimerisation may be substituted. In other words, destabilisation of the homodimerisation interface may be accomplished through the substitution of one or more amino acid residues involved in the homodimerisation interface, including substitution of one or more amino acid residues selected from: V8, V9, L10, A11, S12, S13, T110, Q111, 1112, Y113, V114, 1115, D116, P117, E118 and P119 or at positions equivalent thereto, in the CTLA4 protein (as numbered according to SEQ ID NO: 2).

Formation of covalent bonds, preferably disulphide bonds, between monomeric fusion proteins of the invention may occur via residues (eg cysteine residues) in the first portion of the fusion protein or via residues (eg cysteine residues) in the second portion of the fusion protein, or both.

In one embodiment, the first portion of the fusion protein comprises one or more cysteine residues, capable of forming disulphide bonds to facilitate dimerisation, preferably homodimerisation Preferably, the one or more cysteine residues are located in the C-terminal region of the first portion of the fusion protein, preferably within the C-terminal 25 amino acids of the sequence. Most preferably, the one or more cysteine residues are located within 20 amino acids, 15 amino acids, 10 amino acids or 5 amino acids from the C-terminus of the first portion of the fusion protein.

The one or more cysteine residues may be located within the C-terminal 25 amino acid sequences of any of SEQ ID NO: 1, 2 or 3. For example, the one or more cysteine residues may be located within 20, 15, 10 or 5 amino acids of the C-terminus of any of SEQ ID NO: 1, 2 or 3, or sequences at least 80%, at least 81%, at least 82%, at least 83%, at least 84%, at least 85%, at least 86%, at least 87%, at least 88%, at least 89%, at least 90%, at least 91%, at least 91%, at least 92%, at least 93%, at least 94%, at least 95%, at least 96%, at least 97% at least 98% or at least 99% sequence identity thereto. In the context of SEQ ID NO: 2, disulphide bond formation may occur via the cysteine residue located at residue 120 of SEQ ID NO: 2 (corresponding to C122 of the CTLA4 wild-type sequence), or a position equivalent thereto.

Alternatively, dimerisation, preferably homodimerisation, may be achieved via interactions of the second portion. For example, the second portion may comprise one or more cysteine residues for facilitating homodimerisation. In particularly preferred embodiments, the second portion of the fusion protein comprises a hinge region of an Fc region of an antibody, wherein the hinge region comprises one or more cysteine residues for enabling homodimerisation between two molecules of the fusion protein via the Fc region. Further still homodimersation may be achieved via interactions between the CH3 domain of an Fc region. In certain embodiments, the dimerisation may result from the interaction of fusion proteins of the invention having identical first portions, but differing in the sequence of the second portion comprising an Fc region of an antibody (eg via a “knob in hole” dimerisation).

In a second aspect, the present invention provides a fusion protein comprising:
a first portion comprising or consisting of the amino acid sequence of an extracellular domain of the transmembrane isoform of CTLA4, or functional variant thereof, wherein the sequence comprises an insertion, substitution, or deletion of one or more amino acid residues, located at a position that is within the C-terminal 25 amino acids of the extracellular domain of CTLA4, or region equivalent thereto; and
a second portion comprising the amino acid sequence an Fc region of an antibody.

The extracellular domain of the transmembrane isoform of CTLA4 may comprise the sequence of SEQ ID NO: 2, or is a functional variant or homolog thereof. As such, in a further embodiment of the first aspect of the invention, there is provided a fusion protein comprising:
a first portion comprising a variant CTLA4 as compared to the amino acid sequence set forth in SEQ ID NO: 2; and
a second portion comprising an Fc region of an antibody,
wherein the variant CTLA4 comprises an insertion, substitution, or deletion of one or more amino acid residues at a position between residues 100 and 124 of SEQ ID NO: 2 or position equivalent thereto.

The insertion, substitution, or deletion of one or more amino acid residues in the extracellular domain of CTLA4, or the sequence of SEQ ID NO: 2, preferably forms a flexible domain such that in a further embodiment of the first aspect of the invention, there is provided a fusion protein comprising:
a first portion comprising a variant CTLA4 as compared to the amino acid sequence set forth in SEQ ID NO: 2; and
a second portion comprising an Fc region of an antibody,
wherein the variant CTLA4 comprises a flexible domain located at a position that is between residues 100 to 124 of SEQ ID NO: 2, or position equivalent thereto.

In any embodiment, the insertion, substitution or deletion in the extracellular domain of the transmembrane isoform of CTLA4, or flexible domain, is located at a position between residues 110 and 124 of SEQ ID NO: 2, or a position equivalent thereto. Preferably, the insertion, substitution or deletion in the extracellular domain of the transmembrane isoform of CTLA4, or flexible domain, is located at a position between residues 110 and 120 of SEQ ID NO: 2, or a position equivalent thereto. More preferably, the insertion, substitution or deletion in the extracellular domain of the transmembrane isoform of CTLA4, or flexible domain, is located at a position between residues 115 and 120 of SEQ ID NO: 2, or a position equivalent thereto.

In any embodiment, the insertion, substitution or deletion is of one or more amino acid residues compared to the sequence of the wild-type CTLA4 extracellular domain. An example of the amino acid sequence of the wild-type CTLA extracellular domain is set forth in SEQ ID NO: 2.

In the case of an insertion, typically, the insertion comprises between about 1 to about 30 amino acid residues, optionally wherein the insertion comprises 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues at a position that is between residues 100 to 124 of SEQ ID NO: 2, or position equivalent thereto.

The substitution may be a substitution of one or more amino acid residues. Typically, the number of amino acid substitutions comprises between about 1 to about 30 amino acid residues, optionally wherein the number of amino acid substitutions comprise substitutions of about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues, compared to the sequence of SEQ ID NO: 2 in the region between residues 100 to 124 of SEQ ID NO: 2, or position(s) equivalent thereto.

The deletion may be deletion of one or more amino acid residues. Typically, the number of amino acid deletions comprises between about 1 to about 30 amino acid residues, preferably no more than a deletion of about 1, 2, 3, 4, or 5 amino acids in the region between residues 100 to 124 of SEQ ID NO: 2, or position(s) equivalent thereto.

In certain embodiments, more than one insertion, substitution or deletion may be included in the C-terminal region of the CTLA4, or region between residues 100 to 124 SEQ ID NO: 2, or region equivalent thereto. Indeed, any arrangement may be contemplated provided that the amino acid residues in the C-terminal region of SEQ ID NO: 2 or of the CTLA4, provide for flexibility in this region of the protein compared to a protein having the sequence of SEQ ID NO: 2.

In certain preferred embodiments, the insertion, deletion or substitution is N-terminal to the cysteine residue located at residue 120 of SEQ ID NO: 2 (or equivalent position). Preferably, there is an insertion, substitution or deletion of at least one amino acid residue in the region 20 amino acids N-terminal to the cysteine at residue 120 of SEQ ID NO: 2. In particularly preferred embodiments, insertion, substitution or deletion causes destabilisation of the homodimer interface N-terminal of the cysteine residue located at residue 120 of SEQ ID NO: 2.

In alternative embodiments, the deletion or substitution is of the cysteine residue located at residue 120 of SEQ ID NO: 2 (or equivalent position).

In certain preferred embodiments, the insertion, deletion or substitution is C-terminal to the cysteine residue located at residue 120 of SEQ ID NO: 2 (or equivalent position). Preferably, there is an insertion, substitution or deletion of at least one amino acid residue in the region C-terminal to the cysteine at residue 120 of SEQ ID NO: 2. In particularly preferred embodiments, insertion, substitution or deletion causes destabilisation of the homodimer interface of the CTLA4 sequence.

In certain preferred embodiments, the insertion, substitution or deletion is between or is equivalent to being between residues 100 and 101; between residues 101 and 102; between residues 102 and 103; between residues 103 and 104; between residues 104 and 105; between residues 105 and 106; between residues 106 and 107; between residues 107 and 108; between residues 108 and 109; between residues 109 and 110; between residues 110 and 111; between residues 111 and 112; between residues 112 and 113; between residues 113 and 114; between residues 114 and 115; between residues 115 and 116; between residues 116 and 117; between residues 117 and 118; between residues 118 and 119; between residues 119 and 120 of SEQ ID NO: 1; between residues 120 and 121 of SEQ ID NO: 2; between residues 121 and 122 of SEQ ID NO: 2; between residues 122 and 123 of SEQ ID NO: 2; or between residues 123 and 124 of SEQ ID NO: 2. In certain preferred embodiments, the insertion is between residues 115 and 116 of SEQ ID NO: 2, or position equivalent thereto.

In alternative embodiments, the amino acid substitution may be substitution of a region comprising any one of residues 110, 111, 112, 113, 114, 115, 116, 117, 118, 119, 120, 121, 122, 123 or 124 of SEQ ID NO: 2, or position equivalent thereto. Preferably, the amino acid substitution may be substitution of a region comprising any of residues 110, 111, 112, 113, 114, 115, 116, 117, 118, or 119 of SEQ ID NO: 2, or position equivalent to. In certain embodiments, the substitution may comprise residues 118 and 119 of SEQ ID NO: 2, residues 117 to 119 of SEQ ID NO: 2, residues 116 to 119 of SEQ ID NO: 2, residues 115 to 119 of SEQ ID NO: 2, residues 114 to 119 of SEQ ID NO: 2, residues 113 to 119 of SEQ ID NO: 2, residues 112 to 119 of SEQ ID NO: 2, residues 111 to 119 of SEQ ID NO: 2, residues 110 to 119 of SEQ ID NO: 2, or of positions equivalent thereto.

In a preferred embodiment, the amino acid substitution is of the region comprising residues 116 to 119 of SEQ ID NO: 2 (or region equivalent thereto).

In any embodiment, and regardless of the location of the insertion, substitution or deletion, the insertion, substitution or deletion may be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 or more amino acids.

It will be appreciated that any amino acid or combination of amino acids may be inserted, or included in the flexible domain (e.g., by substitution of the amino acid residues defined in SEQ ID NO: 2). In any embodiment, the insertion or flexible domain is comprised of amino acid residues selected from glycine, serine, alanine, lysine and glutamine residues, preferably glycine, serine and alanine residues.

Similarly, in the context of an amino acid substitution, the amino acids that are substituted preferably comprise any amino acids that facilitate formation of a flexible domain and/or result in destabilisation of the C-terminal region of CTLA4 domain (e.g., the region between residues 100 to 124 of SEQ ID NO: 2, or position equivalent thereto, preferably N-terminal to the cysteine residue at position 120 of SEQ ID NO: 2 or position equivalent thereto). Examples of suitable amino acid residues for substitution include glycine, serine, alanine, lysine and glutamic acid, preferably glycine or serine.

In certain embodiments of the first or second aspects of the invention, the functional variant of the extracellular domain of CTLA4 or the variant CTLA4 retains its capacity to dimerise, for example, via the cysteine residue located at position 120 of SEQ ID NO: 2. Accordingly, in accordance with the first aspect of the invention, the first portion may comprise any variant or functional homolog of the extracellular domain of CTLA4, provided that the polypeptide retains its ability to bind to a ligand of CTLA4, such as B7-1 (CD80) and/or B7-2 (CD86).

Alternatively, in certain embodiments of the first aspect of the invention, the fusion protein is able to dimerise via amino acid residues in the second portion (i.e., in the Fc region of an antibody). For example, the fusion protein may dimerise via cysteine residues present in the second portion.

The functional variant of the extracellular domain of CTLA4, or the variant CTLA4 may comprise or consist of a sequence that is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or at least 99% identical to the amino acid sequence set forth in SEQ ID NO: 1, 2 or 3, wherein the protein retains its ability to bind to a ligand of CTLA4, such as B7-1 (CD80) and/or B7-2 (CD86) and optionally, wherein the protein retains its ability to form a homodimer.

In a third aspect, the present invention also provides a fusion protein comprising:
a first portion comprising the sequence of a soluble isoform of CTLA4; and
a second portion comprising an Fc region of an antibody.

In certain embodiments, the soluble isoform of CTLA4 comprises an amino acid sequence as set forth in SEQ ID NO: 3, or a functional variant or homolog thereof. In one example, the functional variant of SEQ ID NO: 3 may comprise an amino acid sequence that is C terminally truncated by at least 1, at least 2, at least 3, at least 4, at least 5, at least 6, at least 7, at least 8, at least 9 or at least 10 amino acid residues, such as the amino acid sequence of SEQ ID NO: 59.

Preferably, the fusion protein comprises a sequence for facilitating homodimerisation. In particularly preferred examples, dimerisation occurs via the formation of covalent bonds, preferably disulphide bonds, between amino acids in the second portion.

The functional variant or homolog of the soluble isoform of CTLA4 may comprise any variant or functional homolog of soluble CTLA4, provided that the polypeptide retains its ability to bind to a ligand of CTLA4, such as B7-1 (CD80) and/or B7-2 (CD86). The functional variant of the soluble isoform of CTLA4 may comprise or consist of a sequence that is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or at least 99% identical to the amino acid sequence set forth in SEQ ID NO: 3, wherein the protein retains its ability to bind to a ligand of CTLA4, such as B7-1 (CD80) and/or B7-2 (CD86). In one example, the functional variant of SEQ ID NO: 3 may comprise an amino acid sequence that is C terminally truncated by at least 1, at least 2, at least 3, at least 4, at least 5, at least 6, at least 7, at least 8, at least 9 or at least 10 amino acid residues, such as the amino acid sequence of SEQ ID NO: 59.

In any embodiment of the first, second and third aspects, the second portion comprising the sequence of Fc region of an antibody, comprises the amino acid sequence of an Fc region of an immunoglobulin G (Ig), preferably of an IgG1, IgG2, an IgG3 or an IgG4.

Preferably, the second portion comprises the amino acid sequence of an IgG1.

In any embodiment of the first, second and third aspects, the second portion comprises the amino acid sequence of a native Fc region. Preferred native Fc regions of the invention include but are not limited to IgG1, IgG2, IgG3, and IgG4 Fc regions.

In any embodiment of the first, second and third aspects of the invention, the second portion comprises the amino acid sequence of a variant Fc region. In a preferred aspect of the invention, the variant Fc region enhances affinity to the neonatal Fc receptor FcRn. In a most preferred aspect of the invention, the variant Fc region extends half-life of the CTLA4-Ig in vivo. Preferred variants for enhancing FcRn affinity and/or extending half-life in vivo include but are not limited to 2591, 307Q, 308F, 3111, 311V, 378V, 378T, 426V, 428L, 434S, 4361, 436V, 250Q, 434A, 252Y, 254T, and 256E, wherein numbering is according to the EU index. Most preferred variants for enhancing FcRn affinity and/or extending half-life in vivo are 428L and 434S, wherein numbering is according to the EU index.

Preferably, the Fc region (i.e., the second amino acid sequence in the fusion protein) comprises two heavy chain fragments, more preferably the CH2 and CH3 domains of said heavy chain.

In any embodiment of the first, second and third aspects of the invention, the second portion comprises, consists essentially of or consists of an amino acid sequence set forth in any one of SEQ ID NOs: 17 to 24 or 56, or an amino acid sequence having 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence set forth in any one of SEQ ID NOs: 17 to 24 or 56.

In a preferred embodiment of the first and second aspects of the invention, the second portion comprises, consists essentially of or consists of an amino acid sequence set forth in SEQ ID NO: 23, or an amino acid sequence having 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity to the sequence set forth in SEQ ID NO: 23.

In a preferred embodiment of the first and second aspects of the invention, the second portion comprises, consists essentially of or consists of an amino acid sequence set forth in SEQ ID NO: 24, or an amino acid sequence having 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity to the sequence set forth in SEQ ID NO: 24.

In a preferred embodiment of the third aspect of the invention, the second portion comprises, consists essentially of or consists of an amino acid sequence set forth in SEQ ID NO: 25, or an amino acid sequence having 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity the amino acid sequence set forth in SEQ ID NO: 25.

In a preferred embodiment of the third aspect of the invention, the second portion comprises, consists essentially of or consists of an amino acid sequence set forth in SEQ ID NO: 56, or an amino acid sequence having 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity the amino acid sequence set forth in SEQ ID NO: 56.

In any embodiment of the first and second aspects of the invention, the second portion comprises, consists essentially of or consists of an amino acid sequence of any one of SEQ ID NOs: 17 to 24 or 56 having 0 to 8 amino acid insertions, deletions, substitutions or additions (or a combination thereof). In some embodiments, the relevant amino acid sequence may have from 0 to 7, preferably from 0 to 6, preferably from 0 to 5, preferably from 0 to 4, preferably from 0 to 3, preferably from 0 to 2, preferably from 0 to 1 amino acid insertions, deletions, substitutions or additions (or a combination thereof).

In any embodiment of the third aspect of the invention, the second portion comprises, consists essentially of or consists of an amino acid sequence of SEQ ID NO: 25 having with 0 to 8 amino acid insertions, deletions, substitutions or additions (or a combination thereof). In some embodiments, the relevant amino acid sequence may have from 0 to 7, preferably from 0 to 6, preferably from 0 to 5, preferably from 0 to 4, preferably from 0 to 3, preferably from 0 to 2, preferably from 0 to 1 amino acid insertions, deletions, substitutions or additions (or a combination thereof).

In any embodiment of the third aspect of the invention, the second portion comprises, consists essentially of or consists of an amino acid sequence of SEQ ID NO: 56 having with 0 to 8 amino acid insertions, deletions, substitutions or additions (or a combination thereof). In some embodiments, the relevant amino acid sequence may have from 0 to 7, preferably from 0 to 6, preferably from 0 to 5, preferably from 0 to 4, preferably from 0 to 3, preferably from 0 to 2, preferably from 0 to 1 amino acid insertions, deletions, substitutions or additions (or a combination thereof).

In preferred embodiments of any aspects of the invention, the fusion proteins specifically bind CD80/86, preventing the interaction of CD80/86 with CD28, but wherein the cis:CD80:PDL1 interaction remains intact. In other words, the fusion proteins of the invention, enable blocking at CD80/86 without liberating PDL1 relative to abatacept.

In preferred embodiments of any aspect of the invention, a fusion protein as described herein is in the form of a dimer. In the context of the first aspect of the invention, dimerisation may be mediated via the cysteine residue located at residue 120 of SEQ ID NO: 2, or an equivalent position thereto. Alternatively, the dimerisation may be mediated via the second portion of the fusion protein, particularly via a linker sequence included in the second portion for linking the first portion to the second portion or cysteine residues in the second portion. In accordance with the third aspect of the invention, dimerization is preferably mediated via the second portion (i.e., comprising the Fc region of an antibody). In such instances, dimerisation may occur via cysteine residues, and may also be through the use of “knob- and-hole” copies of Fc regions, each fused to identical sequences corresponding to the first portion of the fusion proteins, as described herein. Accordingly, the invention provides for a heterodimer comprising two fusion proteins, wherein the fusion proteins comprise identical first portions corresponding to a CTLA4 ligand binding domain, but wherein the sequences of the second portions of the fusion proteins are complementary and able to interact to form a dimer.

In any aspect of the invention, the first portion of the fusion protein is fused at the C-terminus to second portion in the fusion protein. Alternatively, the first portion of the fusion protein is fused via a linker at the C-terminus to the second portion.

In any aspect, the fusion protein of the present invention includes a peptide linker between the first and second portion (i.e., between the sequence of the CTLA4 variant and the sequence of an Fc of an antibody). In one embodiment, the linker comprises or consists of amino acids. The linker may be any linker known in the art to the skilled person and may be a flexible linker (such as those comprising repeats of glycine and serine residues), a rigid linker (such as those comprising glutamic acid and lysine residues, flanking alanine repeats) and/or a cleavable linker (such as sequences that are susceptible by protease cleavage).

The peptide linker may be any one or more repeats of Gly-Gly-Ser (GGS), Gly-Gly-Gly-Ser (GGGS) or Gly-Gly-Gly-Gly-Ser (GGGGS) or variations thereof. In one embodiment, may comprise or consist of the sequence GGGGSGGGGSGGGGS (G4S)3. In one embodiment, the peptide linker can include the amino acid sequence GGGGS (a linker of 6 amino acids in length) or even longer. The linker may a series of repeating glycine and serine residues (GS) of different lengths, i.e., (GS)n where n is any number from 1 to 15 or more. For example, the linker may be (GS)3 (i.e., GSGSGS) or longer (GS)11 or longer. It will be appreciated that n can be any number including 1, 2, 3, 4, 5, 6, 7, 8, 9, 10, 11 or more.

Linkers used in accordance with the present invention include sequences from the human IgG constant chains, including the C-terminal end of the CH1 domain and upper hinge. Exemplary linker sequences based on the natural IgG isotypes are provided in Table 1 at SEQ ID NOs: 26 to 34. Modified linkers may also be used. An exemplary linker used in the present work is a modified IgG linker (for example an IgG1- or IgG2-based linker) where the cysteines are replaced with serines. However, it will be appreciated that where dimerisation is to be mediated via the linker region, it will be preferably to retain the cysteine residues so as to promote dimerisation.

In preferred embodiments, a fusion protein according to the present invention may have a sequence as shown in any one of SEQ ID NOs: 8 to 15.

In any embodiment, a fusion protein according to the present invention has a sequence as shown in SEQ ID NO: 4.

In any embodiment, a fusion protein according to the present invention has a sequence as shown in any of SEQ ID NOs: 49, 50, 52, 54, 57 or 58.

In any aspect of the invention, there is provided a dimeric protein formed by covalent bonded, preferably disulphide bonded, monomers of a fusion protein described herein.

The present invention also provides nucleic acids, preferably isolated, encoding a fusion protein as described herein. The present invention provides vectors comprising said nucleic acids, optionally, operably linked to control sequences.

The present invention provides host cells containing the vectors, and methods for producing and optionally recovering the fusion proteins.

Further, the present invention provides a pharmaceutical composition comprising a CTLA4-Ig fusion protein as described herein, and a physiologically or pharmaceutically acceptable carrier or diluent.

The present invention further contemplates therapeutic and diagnostic uses for the CTLA4-Ig fusion proteins disclosed herein. The CTLA4-Ig fusion proteins of the invention are preferably used to treat cancer, or a disease, disorder or condition in which immunostimulation is required to elicit treatment. In the most preferred embodiments of the invention, the CTLA4-Ig fusion proteins described herein are used to treat cancer, or an infectious disease.

Accordingly, the present invention provides a method of treating cancer or a condition or disorder requiring immunostimulation in a subject in need thereof, the method comprising administering to said subject, a fusion protein or pharmaceutical composition of the invention, thereby treating cancer or a condition or disorder requiring immunostimulation in the subject.

Further, the present invention provides for a use of a fusion protein as described herein, in the manufacture of a medicament for the treatment of cancer or a condition or disorder requiring immunostimulation in a subject.

The present invention also provides a fusion protein or pharmaceutical composition as described herein, for use in the treatment of cancer or a condition or disorder requiring immunostimulation.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Abatacept liberates PDL1 from surface cis-CD80:PDL1 complexes. (A) Flow cytometry strategy for generating CHO cell clones co-expressing CD80 and PDL1, but with low free PDL1 (MIH1 mAb staining). Shown in red is the CD80^{high}PDL1^{high}/MIH1^{low} sorting gate from which cis-CD80:PDL1 clone 12 and others were derived. (B) Mean fluorescent intensity (MFI) correlation of PD1-Fc binding and MIH1 mAb staining (separate experiments) in 4 different cis-CD80:PDL1 clones. Arrows indicate MFI change upon abatacept treatment. (C) CD28-Fc and abatacept binding to cis-CD80:PDL1 clone 12 cells by mIgG2a or hIgG antibody staining respectively. (D) Free PDL1 (MIH1 staining) on cis-CD80:PDL1 clone 12 cells, either untreated (blue) or treated for 40 mins with CD28-Fc, abatacept, or abatacept pre-incubated with ipilimumab as indicated. (E) MIH1 MFI of cis-CD80:PDL1 clone 12 cells with treatments indicated. Mean±standard deviation (SD), n=3, ***p<0.001, **p<0.01, Student's t-test with Welch's correction. (F) Free PDL1 measured by PD1-Fc binding and MIH1 mAb staining (separate experiments) of untreated (blue) and abatacept treated (red) parental PDL1 clone cells and cis-CD80:PDL1 clone 12 cells. (G) Abatacept dose/response analysis of free PDL1 (MIH1 mAb staining) on cis-CD80:PDL1 clone 12 cells (left) with corresponding abatacept binding (hIgG antibody staining; right). (H) Schematic showing detection of free PDL1 with monoclonal anti-PDL1 antibody MIH1 or PD1-Fc. (I) Schematic showing the binding potential of CD28-Fc and PD1-Fc on surface cis-CD80:PDL1 complexes. (J) Schematic of ipilumumab blockade of abatacept/CTLA4-Fc allowing sequestration of PDL1 by CD80.

FIG. 2. Liberation of surface PDL1 by CTLA4-Ig promotes binding to PD1 cells. (A) Flow cytometry of interactions between CTFR-stained cis-CD80:PDL1 clone 12 cells and CFSE-stained PD1-expressing cells after 1 h co-culture, with Fc fusion and antibody treatments as indicated. Numbers indicate the proportion of events in the interacting cell gate. (B) Proportion of interacting cells from A. Mean±SD, n=3, **p<0.01, Student's t-test with Welch's correction. Schematics of CD28-Fc binding to cell surface cis-CD80:PDL1 complexes and of abatacept-induced trans-PSL1:PD1 cell interactions susceptible to nivolumab. (C) Flow cytometry of interactions between CTFR-stained cis-CD80:PDL1 clone 12 cells and CFSE-stained CD28- or CTLA4-expressing cells or uninfected control cells. The right panel shows PD1-Fc staining of events within the interacting cell gate as indicated. (D) PD1-Fc staining of events within the interacting cell gate shown in C following co-culture of cis-CD80:PDL1 clone 12 cells with CD28- or CTLA4-expressing cells or uninfected control cells, and TGN1412 or ipilimumab as indicated. Mean±SD, n=3, **p<0.01, Student's t-test with Welch's correction. (E) Flow cytometry of interactions between CTFR-stained cis-CD80:PDL1 clone 12 cells and CFSE-stained PD1-expressing cells after 1 h co-culture with unstained third-party CD28- or CTLA4-expressing cells or uninfected control cells (lower left quadrant). Numbers indicate the proportion of events in the interacting cell gate not including (or including) third party cells in the total event count. (F) Proportion of interacting cells as described in E, with TGN1412, ipilimumab, or nivolumab antibody treatments as indicated. Mean±SD, n=3, ****p<0.0001 ***p<0.001, Student's t-test with Welch's correction. (G) Schematics of cis-CD80:PDL1 cell interactions with CD28 cells and of cis-CD80: PSL1 cell interactions with CTLA4 cells resulting in PDL1 liberation and binding to PD1 cells.

FIG. 3. Interaction of CTLA4 expressing cells with cis-CD80:PDL1 cells induces transendocytosis of CD80 but not PDL1. (A) Flow cytometry of CD80mCherry, PDL1mGFP and PD1-Fc staining for a cis-CD80mCherry:PDL1mGFP clone cells, either untreated (blue) or abatacept treated (red). (B) Time course of CD80mCherry, PDL1mGFP, and PD1-Fc staining of cis-CD80mCherry:PDL1mGFP cells during culture with CD28 or CTLA4WT cells. Flow cytometry was gated on singlet cis-CD80mCherry:PDL1mGFP cells to exclude currently-interacting cells. (C) MFI data from the time course shown in B. (D) CD80mCherry (left) and PD1-Fc staining (right) of cis-CD80mCherry:PDL1mGFP cells when untreated (blue) or after 24 h co-culture with CTLA4 cells (red) with ipilimumab added as indicated. (E) CD80mCherry and PD1-Fc MFI from D. Mean±SD, n=3, ****p<0.0001, ***p<0.001, **p<0.01, unpaired t-test with Welch's correction. (F) Free PDL1 assessed by PD1-Fc staining of cis-CD80mCherry:PDL1mGFP cells following 40 mins abatacept (black) or 3 h co-culture with CTLA4^{WT} cells (red).

FIG. 4. Engineered CTLA4-Ig variants block CD80 without liberating PDL1. (A) Schematic of endogenous CTLA4 isoforms. Regions involved in transmembrane CTLA4 homodimerisation are shown as squares, disulphide bonded through Cys122 (equivalent to Cys120 in SEQ ID NO: 2). (B) Engineered CTLA4-Ig fusion proteins. MonoCTLA4-Ig and dimCTLA4-Ig include a human IgG1 Fc domain (gray) with S364N, Y407N, and K409T mutations that prevent dimerisation (monoFc). (C) CTLA4-Ig variant protein alignment showing the C-terminal residues of the CTLA4 ligand-binding domain (bold) followed by divergent sCTLA4 and transmembrane CTLA4 sequences. Disulphide-bonded CTLA4 Cys122 is blue and 4 residues of fused human IgG1 Fc domain are gray. (D) Flow cytometry of cis-CD80:PDL1 cells incubated with CTLA4-Ig fusion proteins (red), showing fusion protein binding (hIgG1 antibody) free PDL1 assessed by MIH1 antibody or PD1-Fc binding, or ipilimumab binding to free ligand-binding domains of cell-bound fusion proteins. Each stain was performed in a different tube to avoid potential crosslinking of monoCTLA4-Ig by anti-IgG1 antibodies. Gated on singlets. (E) Schematic of flexCTLA4-Ig (upper) including a human IgG1 Fc domain (gray) with a C220S mutation but retaining Cys226 and Cys229 to promote interchain disulphide bonding as indicated. Protein sequence (middle) indicates flexCTLA4-Ig Gly insertion site, which is marked by an asterisk in the CTLA4 homodimer structure (lower). Also shown is a schematic model of flexCTLA4-Ig, flex30CTLA4-Ig and flex5CTLA4-Ig fusion proteins showing linker insert lengths approximately to scale. Protein sequence as described in (C) showing linker insert site for the indicated CTLA4-Ig variant proteins. (F) Flow cytometry of cis-CD80:PDL1 cells as described in D following incubation with abatacept, flexCTLA4-Ig, or its corresponding control CTLA4-Ig. (G) Binding of CTLA4-Ig variants to cis-CD80:PDL1 clone 12 cells measured by anti-hIgG1 antibody staining. (H) PDL1 liberation (MIH1 staining) induced by CTLA4-Ig and indicated variants upon binding (hIgG1 staining) to cis-CD80:PDL1 clone 12 cells. In each variant the 118-DPEP-121 peptide in CTLA4 is substituted with GGGS or AKEK as indicated, disabling PDL1 liberation activity. (I) Schematic model of monoCTLA4-Ig binding to a cis-CD80:PDL1 complex. (J) Schematic model showing an equilibrium between monovalent abatacept binding to intact cis-CD80:PDL1 complexes (left) versus bivalent CD80 binding associated with PDL1 liberation (right). (K) Schematic model of homodimeric CTLA4 (abatacept or transmembrane CTLA4) bound to CD80 based on crystal structures of the CTLA4:CD80 complex (left), and a potential model of bivalent flexCTLA4-Ig binding to two cis-CD80:PDL1 complexes (right).

FIG. 5: Binding of CTLA4-Ig variant of the invention to cis-CD80:PDL1 complexes. Association of the fusion protein was tracked over time by anti-IgG1 flow cytometry. In two clones tested, monoCTLA4-Ig dissociation is more rapid, consistent with its inherent monovalent binding.

FIG. 6. FlexCTLA4-Ig variants induce graded mPD-L1 liberation from mouse cis-mCD80: mPD-L1 complexes. (A) Flow cytometry of CHO mPD-L1+mCD80 cells (left) or 24 h LPS-treated primary mouse splenic DCs (right) incubated with CTLA4-Ig variants (red) showing free mPD-L1 assessed by MIH6 antibody. (B) Relative MFI of free PD-L1 on CHO mPD-L1+mCD80 cells treated with CTLA4-Ig variants with different linker insert lengths (aa), with abatacept and CTLA4-Ig controls indicated. (C) Schematic model of CTLA4-induced PD-L1 liberation in mouse and human. (D-E) Schematic models of interactions between CTLA4-Ig variants and human cis-CD80:PDL1 complexes (D) or mouse cis-mCD80: mPDL1 complexes (E). For human (D), bivalent FlexCTLA4-Ig binding to two cis-CD80:PD-L1 complexes does not displace PDL1. For mouse (E), FlexCTLA4-Ig binding to cis-mCD80: mPD-L1 complexes partially displaces PDL1, potentially by disrupting only a single cis-mCD80: mPD-L1 complex upon CD80 crosslinking.

FIG. 7. PD-L1 liberation is essential for immunosuppression by CTLA4-Ig in vivo. (A) CIA schematic showing CTLA4-Ig variant treatment schedule. (B) Arthritis measured by clinical index and ankle thickening over time. Mean±SEM, n=9, p values versus vehicle, two-way analysis of variance (ANOVA) with Tukey's multiple comparisons test. Control CTLA4-Ig versus abatacept p=0.13. (C) CIA phenotypes at endpoint. Left to right: final arthritis score; percentage of Tfh and Treg cells in the splenic CD4⁺ population; percentage of plasma cells (PCs); percentage of germinal centre (GC) B cells in the CD19+ population. Mean±SD, n=9, *p<0.05, **p<0.01, ***p<0.001, ****p<0.0001, unpaired t-test. (D) AIA schematic showing CTLA4-Ig variant treatment schedule. (E) AIA phenotypes at endpoint. Left to right: joint CD45⁺ infiltration by cell count, percentage of PCs, and percentage of GC B cells in the CD19+ population. Mean±SD, n=6, *p<0.05, **p<0.01, unpaired t-test. (F) Collagen-induced arthritis score (mean+/− standard error, n=8 mice per group) for the indicated treatment groups (Flex5 CTLA4-Ig^{LALAPG}, S4d10 CTLA4-Ig^{LALAPG}, vehicle, control CTLA4-Ig and abatacept).

FIG. 8. FlexCTLA4-Ig triggers immune activation in naïve mice. (A) Schematic of naïve wild type mouse treatment. (B) Naïve mouse treatment phenotypes at endpoint. Left to right: percentage CXCR5⁺ cells among CD4⁺ splenocytes; percentage of PCs; percentage of GC B cells in the CD19+ population. (C) Flow cytometry of total mPDL1 (MIH5 staining) on total splenocytes, showing expression profiles of representative mice following treatment. Mean±SD, n=3, *p<0.05, unpaired t-test. (D) Flow cytometry of mCD80 (16-10A1 staining) in Cd11c⁺MHCII⁺ DCs from mice following treatment. Mean±SD, n=3, *p<0.05, **p<0.01, unpaired t-test. (E) CD80mCherry, PD-L1mGFP, and free surface PD-L1 (PD1-Fc binding) on cis-CD80mCherry:PD-L1mGFP cells incubated with the indicated CTLA4-Ig variants during culture with (red) or without (blue) CTLA4WT cells. Flow cytometry was gated on singlet cis-CD80mCherry:PD-L1mGFP cells to exclude currently-interacting cells. (F) Percentage of cells cis-CD80mCherry:PD-L1mGFP cells from (E) with free PD-L1 (PD1-Fc⁺) and the corresponding relative change in CD80mCherry protein MFI. Control CD28 cell co-incubation is indicated (green). Mean±SD, n=3, *p<0.05, paired t-test.

FIG. 9. (A) Binding of abatacept (CTLA4-Ig), Y100F-CTLA4-Ig, and Y100F-Flex-CTLA4-Ig to CHO cells expressing mouse mCD80 or mCD86 (left) or human hCD80 or hCD86 (right). Across both species the Y100F and Y100F-Flex variants specifically bind CD80 proteins but not CD86 proteins. (B) Free PD-L1 (MIH1 staining) on CHO cis-CD80:PD-L1 cells treated with control CTLA4-Ig, FlexCTLA4-Ig, Y100F-CTLA4-Ig, or Y100F-FlexCTLA4-Ig. The ‘wild type’ and Y100F versions of CTLA4-Ig both trigger PD-L1 liberation, whereas both versions of FlexCTLA4-Ig fail to liberate PD-L1.

FIG. 10. FlexCTLA4-Ig triggers anti-tumor immunity. (A) Schematic of MC38 tumor mouse treatments. (B) MC38 tumor volume (mm³). Mean±standard error, n=10 mice per group, Flex5CTLA4-Ig versus vehicle p=0.001, atezolizumab versus vehicle p<0.0001, Flex5CTLA4-Ig versus atezolizumab p=0.39. Two-way ANOVA with Tukey's multiple comparisons test. (C) Tumor growth of the 10 individual mice within each group shown in B.

Sequence information

| | | SEQ ID |
|---|---|---|
| Description | Sequence | NO: |
| CTLA4 | MHVAQPAVVLASSRGIASFVCEYASPGKATEVRVTVL | 1 |
| CD80/CD86 | RQADSQVTEVCAATYMMGNELTFLDDSICTGTSSGN | |
| ligand binding | QVNLTIQGLRAMDTGLYICKVELMYPPPYYLGIGNGTQ | |
| domain | IYVI | |
| Residues | | |
| involved in | | |
| homo- | | |
| dimerisation | | |
| interface | | |
| underlined | | |
| CTLA4 | MHVAQPAVVLASSRGIASFVCEYASPGKATEVRVTVL | 2 |
| extracellular | RQADSQVTEVCAATYMMGNELTFLDDSICTGTSSGN | |
| domain (ECD) | QVNLTIQGLRAMDTGLYICKVELMYPPPYYLGIGNGTQ | |
| mature | IYVIDPEPCPDSD | |
| sequence | | |
| Residues | | |
| involved in | | |
| homo- | | |
| dimerisation | | |
| interface | | |
| underlined | | |
| CTLA4 soluble | MHVAQPAVVLASSRGIASFVCEYASPGKATEVRVTVL | 3 |
| isoform- | RQADSQVTEVCAATYMMGNELTFLDDSICTGTSSGN | |
| mature | QVNLTIQGLRAMDTGLYICKVELMYPPPYYLGIGNGTQ | |
| sequence | IYVIAKEKKPSYNRGLCENAPNRARM | |
| MonoCTLA4-Ig | MHVAQPAVVLASSRGIASFVCEYASPGKATEVRVTVL | 4 |
| | RQADSQVTEVCAATYMMGNELTFLDDSICTGTSSGN | |
| | QVNLTIQGLRAMDTGLYICKVELMYPPPYYLGIGNGTQ | |
| | IYVIAKEKKPSYNRGLCENAPNRARMQEPKSSDKTHT | |
| | SPPSPAPELLGGSSVFLFPPKPKDTLMISRTPEVTCVV | |
| | VDVSHEDPEVKFNWYVDGVEVHNAKTKPREEQYNST | |
| | YRVVSVLTVLHQDWLNGKEYKCKVSNKALPAPIEKTIS | |
| | KAKGQPREPQVYTLPPSRDELTKNQVNLTCLVKGFYP | |
| | SDIAVEWESNGQPENNYKTTPPVLDSDGSFFLNSTLT | |
| | VDKSRWQQGNVFSCSVMHEALHNHYTQKSLSLSPGK | |
| DimCTLA4-Ig | MHVAQPAVVLASSRGIASFVCEYASPGKATEVRVTVL | 5 |
| [control for | RQADSQVTEVCAATYMMGNELTFLDDSICTGTSSGN | |
| monoCTLA4-Ig] | QVNLTIQGLRAMDTGLYICKVELMYPPPYYLGIGNGTQ | |
| | IYVIDPEPCPDSDQEPKSSDKTHTSPPSPAPELLGGSS | |
| | VFLFPPKPKDTLMISRTPEVTCVVVDVSHEDPEVKFN | |
| | WYVDGVEVHNAKTKPREEQYNSTYRVVSVLTVLHQD | |
| | WLNGKEYKCKVSNKALPAPIEKTISKAKGQPREPQVY | |
| | TLPPSRDELTKNQVNLTCLVKGFYPSDIAVEWESNGQ | |
| | PENNYKTTPPVLDSDGSFFLNSTLTVDKSRWQQGNV | |
| | FSCSVMHEALHNHYTQKSLSLSPGK | |
| Control CTLA4- | MHVAQPAVVLASSRGIASFVCEYASPGKATEVRVTVL | 6 |
| Ig [control for all | RQADSQVTEVCAATYMMGNELTFLDDSICTGTSSGN | |
| flexCTLA4-Ig | QVNLTIQGLRAMDTGLYICKVELMYPPPYYLGIGNGTQ | |
| variants] | IYVIDPEPCPDSDQEPKSSDKTHTCPPCPAPELLGGSS | |
| | VFLFPPKPKDTLMISRTPEVTCVVVDVSHEDPEVKFN | |
| | WYVDGVEVHNAKTKPREEQYNSTYRVVSVLTVLHQD | |
| | WLNGKEYKCKVSNKALPAPIEKTISKAKGQPREPQVY | |
| | TLPPSRDELTKNQVSLTCLVKGFYPSDIAVEWESNGQ | |
| | PENNYKTTPPVLDSDGSFFLYSKLTVDKSRWQQGNV | |
| | FSCSVMHEALHNHYTQKSLSLSPGK | |
| FlexCTLA4-Ig | MHVAQPAVVLASSRGIASFVCEYASPGKATEVRVTVL | 7 |
| [could also be | RQADSQVTEVCAATYMMGNELTFLDDSICTGTSSGN | |
| called | QVNLTIQGLRAMDTGLYICKVELMYPPPYYLGIGNGTQ | |
| ‘flex1CTLA4-Ig'] | IYVIGDPEPCPDSDQEPKSSDKTHTCPPCPAPELLGG | |
| | SSVFLFPPKPKDTLMISRTPEVTCVVVDVSHEDPEVKF | |
| | NWYVDGVEVHNAKTKPREEQYNSTYRVVSVLTVLHQ | |
| | DWLNGKEYKCKVSNKALPAPIEKTISKAKGQPREPQV | |
| | YTLPPSRDELTKNQVSLTCLVKGFYPSDIAVEWESNG | |
| | QPENNYKTTPPVLDSDGSFFLYSKLTVDKSRWQQGN | |
| | VFSCSVMHEALHNHYTQKSLSLSPGK | |
| Flex2CTLA4-Ig | MHVAQPAVVLASSRGIASFVCEYASPGKATEVRVTVL | 8 |
| | RQADSQVTEVCAATYMMGNELTFLDDSICTGTSSGN | |
| | QVNLTIQGLRAMDTGLYICKVELMYPPPYYLGIGNGTQ | |
| | IYVIGSDPEPCPDSDQEPKSSDKTHTCPPCPAPELLG | |
| | GSSVFLFPPKPKDTLMISRTPEVTCVVVDVSHEDPEVK | |
| | FNWYVDGVEVHNAKTKPREEQYNSTYRVVSVLTVLH | |
| | QDWLNGKEYKCKVSNKALPAPIEKTISKAKGQPREPQ | |
| | VYTLPPSRDELTKNQVSLTCLVKGFYPSDIAVEWESN | |
| | GQPENNYKTTPPVLDSDGSFFLYSKLTVDKSRWQQG | |
| | NVFSCSVMHEALHNHYTQKSLSLSPGK | |
| Flex3CTLA4-Ig | MHVAQPAVVLASSRGIASFVCEYASPGKATEVRVTVL | 9 |
| | RQADSQVTEVCAATYMMGNELTFLDDSICTGTSSGN | |
| | QVNLTIQGLRAMDTGLYICKVELMYPPPYYLGIGNGTQ | |
| | IYVIGGSDPEPCPDSDQEPKSSDKTHTCPPCPAPELL | |
| | GGSSVFLFPPKPKDTLMISRTPEVTCVVVDVSHEDPE | |
| | VKFNWYVDGVEVHNAKTKPREEQYNSTYRVVSVLTV | |
| | LHQDWLNGKEYKCKVSNKALPAPIEKTISKAKGQPRE | |
| | PQVYTLPPSRDELTKNQVSLTCLVKGFYPSDIAVEWE | |
| | SNGQPENNYKTTPPVLDSDGSFFLYSKLTVDKSRWQ | |
| | QGNVFSCSVMHEALHNHYTQKSLSLSPGK | |
| Flex5CTLA4-Ig | MHVAQPAVVLASSRGIASFVCEYASPGKATEVRVTVL | 10 |
| | RQADSQVTEVCAATYMMGNELTFLDDSICTGTSSGN | |
| | QVNLTIQGLRAMDTGLYICKVELMYPPPYYLGIGNGTQ | |
| | IYVIGGGGSDPEPCPDSDQEPKSSDKTHTCPPCPAPE | |
| | LLGGSSVFLFPPKPKDTLMISRTPEVTCVVVDVSHEDP | |
| | EVKFNWYVDGVEVHNAKTKPREEQYNSTYRVVSVLT | |
| | VLHQDWLNGKEYKCKVSNKALPAPIEKTISKAKGQPR | |
| | EPQVYTLPPSRDELTKNQVSLTCLVKGFYPSDIAVEW | |
| | ESNGQPENNYKTTPPVLDSDGSFFLYSKLTVDKSRW | |
| | QQGNVFSCSVMHEALHNHYTQKSLSLSPGK | |
| Flex10CTLA4- | MHVAQPAVVLASSRGIASFVCEYASPGKATEVRVTVL | 11 |
| Ig | RQADSQVTEVCAATYMMGNELTFLDDSICTGTSSGN | |
| | QVNLTIQGLRAMDTGLYICKVELMYPPPYYLGIGNGTQ | |
| | IYVIGGGGSGGGGSDPEPCPDSDQEPKSSDKTHTCP | |
| | PCPAPELLGGSSVFLFPPKPKDTLMISRTPEVTCVVVD | |
| | VSHEDPEVKFNWYVDGVEVHNAKTKPREEQYNSTYR | |
| | VVSVLTVLHQDWLNGKEYKCKVSNKALPAPIEKTISKA | |
| | KGQPREPQVYTLPPSRDELTKNQVSLTCLVKGFYPSD | |
| | IAVEWESNGQPENNYKTTPPVLDSDGSFFLYSKLTVD | |
| | KSRWQQGNVFSCSVMHEALHNHYTQKSLSLSPGK | |
| Flex15CTLA4- | MHVAQPAVVLASSRGIASFVCEYASPGKATEVRVTVL | 12 |
| Ig | RQADSQVTEVCAATYMMGNELTFLDDSICTGTSSGN | |
| | QVNLTIQGLRAMDTGLYICKVELMYPPPYYLGIGNGTQ | |
| | IYVIGGGGSGGGGSGGGGSDPEPCPDSDQEPKSSDK | |
| | THTCPPCPAPELLGGSSVFLFPPKPKDTLMISRTPEVT | |
| | CVVVDVSHEDPEVKFNWYVDGVEVHNAKTKPREEQY | |
| | NSTYRVVSVLTVLHQDWLNGKEYKCKVSNKALPAPIE | |
| | KTISKAKGQPREPQVYTLPPSRDELTKNQVSLTCLVK | |
| | GFYPSDIAVEWESNGQPENNYKTTPPVLDSDGSFFLY | |
| | SKLTVDKSRWQQGNVFSCSVMHEALHNHYTQKSLSL | |
| | SPGK | |
| Flex20CTLA4- | MHVAQPAVVLASSRGIASFVCEYASPGKATEVRVTVL | 13 |
| Ig | ROADSQVTEVCAATYMMGNELTFLDDSICTGTSSGN | |
| | QVNLTIQGLRAMDTGLYICKVELMYPPPYYLGIGNGTQ | |
| | IYVIGGGGSGGGGSGGGGSGGGGSDPEPCPDSDQE | |
| | PKSSDKTHTCPPCPAPELLGGSSVFLFPPKPKDTLMIS | |
| | RTPEVTCVVVDVSHEDPEVKFNWYVDGVEVHNAKTK | |
| | PREEQYNSTYRVVSVLTVLHQDWLNGKEYKCKVSNK | |
| | ALPAPIEKTISKAKGQPREPQVYTLPPSRDELTKNQVS | |
| | LTCLVKGFYPSDIAVEWESNGQPENNYKTTPPVLDSD | |
| | GSFFLYSKLTVDKSRWQQGNVFSCSVMHEALHNHYT | |
| | QKSLSLSPGK | |
| Flex25CTLA4- | MHVAQPAVVLASSRGIASFVCEYASPGKATEVRVTVL | 14 |
| Ig | RQADSQVTEVCAATYMMGNELTFLDDSICTGTSSGN | |
| | QVNLTIQGLRAMDTGLYICKVELMYPPPYYLGIGNGTQ | |
| | IYVIGGGGSGGGGSGGGGSGGGGSGGGGSDPEPCP | |
| | DSDQEPKSSDKTHTCPPCPAPELLGGSSVFLFPPKPK | |
| | DTLMISRTPEVTCVVVDVSHEDPEVKFNWYVDGVEVH | |
| | NAKTKPREEQYNSTYRVVSVLTVLHQDWLNGKEYKC | |
| | KVSNKALPAPIEKTISKAKGQPREPQVYTLPPSRDELT | |
| | KNQVSLTCLVKGFYPSDIAVEWESNGQPENNYKTTPP | |
| | VLDSDGSFFLYSKLTVDKSRWQQGNVFSCSVMHEAL | |
| | HNHYTQKSLSLSPGK | |
| Flex30CTLA4- | MHVAQPAVVLASSRGIASFVCEYASPGKATEVRVTVL | 15 |
| Ig | RQADSQVTEVCAATYMMGNELTFLDDSICTGTSSGN | |
| | QVNLTIQGLRAMDTGLYICKVELMYPPPYYLGIGNGTQ | |
| | IYVIGGGGSGGGGSGGGGSGGGGSGGGGSGGGGS | |
| | DPEPCPDSDQEPKSSDKTHTCPPCPAPELLGGSSVFL | |
| | FPPKPKDTLMISRTPEVTCVVVDVSHEDPEVKFNWYV | |
| | DGVEVHNAKTKPREEQYNSTYRVVSVLTVLHQDWLN | |
| | GKEYKCKVSNKALPAPIEKTISKAKGQPREPQVYTLPP | |
| | SRDELTKNQVSLTCLVKGFYPSDIAVEWESNGQPENN | |
| | YKTTPPVLDSDGSFFLYSKLTVDKSRWQQGNVFSCSV | |
| | MHEALHNHYTQKSLSLSPGK | |
| Abatacept | MHVAQPAVVLASSRGIASFVCEYASPGKATEVRVTVL | 16 |
| [note: mature | RQADSQVTEVCAATYMMGNELTFLDDSICTGTSSGN | |
| protein | QVNLTIQGLRAMDTGLYICKVELMYPPPYYLGIGNGTQ | |
| sequence, | IYVIDPEPCPDSDQEPKSSDKTHTSPPSPAPELLGGSS | |
| without N- | VFLFPPKPKDTLMISRTPEVTCVVVDVSHEDPEVKFN | |
| terminal signal | WYVDGVEVHNAKTKPREEQYNSTYRVVSVLTVLHQD | |
| peptide] | WLNGKEYKCKVSNKALPAPIEKTISKAKGQPREPQVY | |
| | TLPPSRDELTKNQVSLTCLVKGFYPSDIAVEWESNGQ | |
| | PENNYKTTPPVLDSDGSFFLYSKLTVDKSRWQQGNV | |
| | FSCSVMHEALHNHYTQKSLSLSPGK | |
| Fc(IgG1) (EU | PAPELLGGPSVFLFPPKPKDTLMISRTPEVTCWVDVS | 17 |
| numbering 230- | HEDPEVKFNWYVDGVEVHNAKTKPREEQYNSTYRVV | |
| 447) | SVLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| Fc(IgG2) (EU | PAPPVAGPSVFLFPPKPKDTLMISRTPEVTCVVVDVSH | 18 |
| numbering 230- | EDPEVQFNWYVDGVEVHNAKTKPREEQFNSTFRVVS | |
| 447) | VLTVVHQDWLNGKEYKCKVSNKGLPAPIEKTISKTKG | |
| | QPREPQVYTLPPSREEMTKNQVSLTCLVKGFYPSDIA | |
| | VEWESNGQPENNYKTTPPMLDSDGSFFLYSKLTVD | |
| | KSRWQQGNVFSCSVMHEALHNHYTQKSLSLSPGK | |
| Fc(IgG3) (EU | PAPELLGGPSVFLFPPKPKDTLMISRTPEVTCVVVDVS | 19 |
| numbering 230- | HEDPEVQFKWYVDGVEVHNAKTKPREEQYNSTFRVV | |
| 447) | SVLTVLHQDWLNGKEYKCKVSNKALPAPIEKTISKTKG | |
| | QPREPQVYTLPPSREEMTKNQVSLTCLVKGFYPSDIA | |
| | VEWESSGQPENNYNTTPPMLDSDGSFFLYSKLTVDK | |
| | SRWQQGNIFSCSVMHEALHNRFTQKSLSLSPGK | |
| Fc(IgG4) (EU | PAPEFLGGPSVFLFPPKPKDTLMISRTPEVTCVVVDVS | 20 |
| numbering 230- | QEDPEVQFNWYVDGVEVHNAKTKPREEQFNSTYRVV | |
| 447) | SVLTVLHQDWLNGKEYKCKVSNKGLPSSIEKTISKAKG | |
| | QPREPQVYTLPPSQEEMTKNQVSLTCLVKGFYPSDIA | |
| | VEWESNGQPENNYKTTPPVLDSDGSFFLYSRLTVDKS | |
| | RWQEGNVFSCSVMHEALHNHYTQKSLSLSLGK | |
| Fc(IgG1-238S) | PAPELLGGSSVFLFPPKPKDTLMISRTPEVTCVVVDVS | 21 |
| (EU numbering | HEDPEVKFNWYVDGVEVHNAKTKPREEQYNSTYRVV | |
| 230-447) | SVLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| Fc(IgG2- | PAPELLGGPSVFLFPPKPKDTLMISRTPEVTCWVDVS | 22 |
| 233E/234L/235L/ | HEDPEVQFNWYVDGVEVHNAKTKPREEQFNSTFRVV | |
| 236G) (EU | SVLTVVHQDWLNGKEYKCKVSNKGLPAPIEKTISKTKG | |
| numbering 230- | QPREPQVYTLPPSREEMTKNQVSLTCLVKGFYPSDIA | |
| 447) | VEWESNGQPENNYKTTPPMLDSDGSFFLYSKLTVDK | |
| | SRWQQGNVFSCSVMHEALHNHYTQKSLSLSPGK | |
| Fc(IgG2- | PAPELLGGSSVFLFPPKPKDTLMISRTPEVTCVVVDVS | 23 |
| 233E/234L/235L/ | HEDPEVQFNWYVDGVEVHNAKTKPREEQFNSTFRVV | |
| 236G/238S) | SVLTVVHQDWLNGKEYKCKVSNKGLPAPIEKTISKTKG | |
| (EU numbering | QPREPQVYTLPPSREEMTKNQVSLTCLVKGFYPSDIA | |
| 230-447) | VEWESNGQPENNYKTTPPMLDSDGSFFLYSKLTVDK | |
| | SRWQQGNVFSCSVMHEALHNHYTQKSLSLSPGK | |
| Fc used in this | PAPELLGGSSVFLFPPKPKDTLMISRTPEVTCVVVDVS | 24 |
| work (first | HEDPEVKFNWYVDGVEVHNAKTKPREEQYNSTYRVV | |
| aspect) | SVLTVLHQDWLNGKEYKCKVSNKALPAPIEKTISKAKG | |
| | QPREPQVYTLPPSRDELTKNQVSLTCLVKGFYPSDIAV | |
| | EWESNGQPENNYKTTPPVLDSDGSFFLYSKLTVDKSR | |
| | WQQGNVFSCSVMHEALHNHYTQKSLSLSPGK | |
| Fc used in this | PAPELLGGSSVFLFPPKPKDTLMISRTPEVTCVVVDVS | 25 |
| work (second | HEDPEVKFNWYVDGVEVHNAKTKPREEQYNSTYRVV | |
| aspect) | SVLTVLHQDWLNGKEYKCKVSNKALPAPIEKTISKAKG | |
| | QPREPQVYTLPPSRDELTKNQVNLTCLVKGFYPSDIA | |
| | VEWESNGQPENNYKTTPPVLDSDGSFFLNSTLTVDKS | |
| | RWQQGNVFSCSVMHEALHNHYTQKSLSLSPGK | |
| Linker(IgG1) | EPKSCDKTHTCPPC | 26 |
| (EU numbering | | |
| 216-229) | | |
| Linker(IgG2) | ERKCCVECPPC | 27 |
| (EU numbering | | |
| 216-229) | | |
| Linker(IgG3) | ELKTPLGDTTHTCPRCPEPKSCDTPPPCPRCPEPKSC | 28 |
| (EU numbering | DTPPPCPRCPEPKSCDTPPPCPRC | |
| 216-229) | | |
| Linker(IgG4) | ESKYGPPCPSC | 29 |
| (EU numbering | | |
| 216-229) | | |
| Linker(IgG1- | EPKSSDKTHTSPPS | 30 |
| 220S/226S/229S) | | |
| (EU | | |
| numbering 216- | | |
| 229) | | |
| Linker(IgG2- | ERKSSVESPPS | 31 |
| 219S/220S/226S/ | | |
| 229S) | | |
| Linker(Q-IgG1- | QEPKSSDKTHTSPPS | 32 |
| 220S/226S/229S) | | |
| [used for | | |
| ″monoCTLA4- | | |
| Ig″ | | |
| Linker(Q-IgG2- | QERKSSVESPPS | 33 |
| 219S/220S/226S/ | | |
| 229S) | | |
| Linker used in | QEPKSSDKTHTCPPC | 34 |
| Flex CTL4A-Ig | | |
| constructs | | |
| Primer mCD80 | TTCTCTAGGCGCCGGAATTCGCCACCATGGCTTGC | 35 |
| (NM_001359898.1): | AATTG | |
| forward | | |
| Primer mCD80 | CGGAATTGATCCCGCTCGAGCTAAAGGAAGACGGT | 36 |
| (NM_001359898.1): | CTGTTCAGC | |
| reverse | | |
| Primer mPDL1 | TGTATCGAATTCATGAGGATATTTGCTGGCATTATAT | 37 |
| (NM_021893.3): | TCACAGCC | |
| forward | | |
| Primer mPDL1 | TCTATCCTCGAGTTACGTCTCCTCGAATTGTGTATC | 38 |
| (NM_021893.3): | ATTTCGG | |
| reverse | | |
| hCD80 | ATTACAGAATTCGCCACCATGGGCCACACACG | 39 |
| (NM_005191.3): | | |
| forward | | |
| hCD80 | CTGAGACTCGAGTTATACAGGGCGTACACTTTCC | 40 |
| (NM_005191.3): | | |
| reverse | | |
| hPDL1 | TAAGCAGAATTCGCCACCATGAGGATATTTGCTGTC | 41 |
| (NM_014143.3): | TTTATATTC | |
| forward | | |
| hPDL1 | TAAGCACTCGAGTTACGTCTCCTCCAAATGTGTATC | 42 |
| (NM_014143.3): | | |
| reverse | | |
| hPD1 | TAAGCAGAATTCGCCACCATGCAGATCCCACAGGC | 43 |
| (NM_005018.2): | G | |
| forward | | |
| hPD1 | TAAGCACTCGAGTCAGAGGGGCCAAGAG | 44 |
| (NM_005018.2): | | |
| reverse | | |
| hCD28 | TAAGCAGAATTCGCCACCATGCTCAGGCTGCTCTTG | 45 |
| (NM_006139.3): | | |
| forward | | |
| hCD28 | CCTGTACTCGAGTCAGGAGCGATAGGCTGC | 46 |
| (NM_006139.3): | | |
| reverse | | |
| hCTLA4 | GTTCTAGAATTCGCCACCATGGCTTGCCTTGGATTT | 47 |
| (NM_005214.4 | C | |
| with SNP | | |
| rs231775 in | | |
| signal peptide): | | |
| forward | | |
| hCTLA4 | CCTGTACTCGAGTCAATTGATGGGAATAAAATAAGG | 48 |
| (NM_005214.4 | CTGAAATTG | |
| with SNP | | |
| rs231775 in | | |
| signal peptide): | | |
| reverse | | |
| Example of a | MHVAQPAVVLASSRGIASFVCEYASPGKATEVRVTVL | 49 |
| substitution in | RQADSQVTEVCAATYMMGNELTFLDDSICTGTSSGN | |
| the region N- | QVNLTIQGLRAMDTGLYICKVELMYPPPYYLGIGNGTQ | |
| terminal to | IYVIGGGSCPDSDQEPKSSDKTHTCPPCPAPELLGGS | |
| cysteine at | SVFLFPPKPKDTLMISRTPEVTCVVVDVSHEDPEVKFN | |
| position 120 of | WYVDGVEVHNAKTKPREEQYNSTYRVVSVLTVLHQD | |
| SEQ ID NO: 2 | WLNGKEYKCKVSNKALPAPIEKTISKAKGQPREPQVY | |
| (″DPEP″ | TLPPSRDELTKNQVSLTCLVKGFYPSDIAVEWESNGQ | |
| substitution to | PENNYKTTPPVLDSDGSFFLYSKLTVDKSRWQQGNV | |
| GGGS shown in | FSCSVMHEALHNHYTQKSLSLSPGK | |
| bold) | | |
| Example of a | MHVAQPAVVLASSRGIASFVCEYASPGKATEVRVTVL | 50 |
| substitution in | RQADSQVTEVCAATYMMGNELTFLDDSICTGTSSGN | |
| the region N- | QVNLTIQGLRAMDTGLYICKVELMYPPPYYLGIGNGTQ | |
| terminal to | IYVIAKEKCPDSDQEPKSSDKTHTCPPCPAPELLGGS | |
| cysteine at | SVFLFPPKPKDTLMISRTPEVTCVVVDVSHEDPEVKFN | |
| position 120 of | WYVDGVEVHNAKTKPREEQYNSTYRVVSVLTVLHQD | |
| SEQ ID NO: 2 | WLNGKEYKCKVSNKALPAPIEKTISKAKGQPREPQVY | |
| | TLPPSRDELTKNQVSLTCLVKGFYPSDIAVEWESNGQ | |
| | PENNYKTTPPVLDSDGSFFLYSKLTVDKSRWQQGNV | |
| | FSCSVMHEALHNHYTQKSLSLSPGK | |
| (″DPEP″ | GCAATGCACGTGGCCCAGCCTGCTGTGGTACTGGC | 51 |
| substitution to | CAGCAGCCGAGGCATCGCCAGCTTTGTGTGTGAGT | |
| AKEK shown in | ATGCATCTCCAGGCAAAGCCACTGAGGTCCGGGTG | |
| bold) | ACAGTGCTTCGGCAGGCTGACAGCCAGGTGACTGA | |
| LALA-CTLA4-Ig | AGTCTGTGCGGCAACCTACATGATGGGGAATGAGT | |
| nucleic acid | TGACCTTCCTAGATGATTCCATCTGCACGGGCACCT | |
| sequence | CCAGTGGAAATCAAGTGAACCTCACTATCCAAGGAC | |
| | TGAGGGCCATGGACACGGGACTCTACATCTGCAAG | |
| | GTGGAGCTCATGTACCCACCGCCATACTACCTGGG | |
| | CATAGGCAACGGAACCCAGATTTATGTAATTGATCC | |
| | AGAACCGTGCCCAGATTCTGACCAGGAGCCCAAGA | |
| | GCAGCGACAAGACCCACACCTGCCCTCCTTGCCCT | |
| | GCTCCAGAGGCTGCTGGCGGCCCTTCCGTGTTCCT | |
| | GTTTCCCCCTAAGCCAAAGGACACCCTGATGATCAG | |
| | CCGGACCCCAGAGGTGACATGCGTGGTGGTGGAC | |
| | GTGTCCCACGAGGACCCCGAGGTGAAGTTCAACTG | |
| | GTACGTGGATGGCGTGGAGGTGCACAATGCCAAGA | |
| | CAAAGCCTCGGGAGGAGCAGTACAACAGCACCTAT | |
| | AGAGTGGTGTCCGTGCTGACAGTGCTGCACCAGGA | |
| | CTGGCTGAACGGCAAGGAGTACAAGTGCAAGGTGT | |
| | CTAATAAGGCCCTGCCCGCCCCTATCGAGAAGACC | |
| | ATCAGCAAGGCAAAGGGACAGCCAAGGGAGCCACA | |
| | GGTGTATACACTGCCACCCTCTCGCGACGAGCTGA | |
| | CCAAGAACCAAGTGAGCCTGACATGTCTGGTGAAG | |
| | GGCTTCTACCCCTCCGACATCGCCGTGGAGTGGGA | |
| | GTCTAATGGCCAGCCTGAGAACAATTATAAGACCAC | |
| | ACCTCCAGTGCTGGACTCCGATGGCTCTTTTTTCT | |
| | GTACTCTAAGCTGACAGTGGATAAGAGCAGGTGGC | |
| | AGCAGGGCAACGTGTTCAGCTGTAGCGTGATGCAC | |
| | GAAGCCCTGCACAATCACTACACTCAGAAGAGCCT | |
| | GTCCCTGTCACCTGGAAAATGA | |
| CTLA4-Ig^{LALA} | MHVAQPAVVLASSRGIASFVCEYASPGKATEVRVTVL | 52 |
| (amino acid | RQADSQVTEVCAATYMMGNELTFLDDSICTGTSSGN | |
| sequence) | QVNLTIQGLRAMDTGLYICKVELMYPPPYYLGIGNGTQ | |
| CTLA4 | IYVIDPEPCPDSDQEPKSSDKTHTCPPCPAPEAAGGP | |
| sequence in | SVFLFPPKPKDTLMISRTPEVTCVVVDVSHEDPEVKFN | |
| italics | WYVDGVEVHNAKTKPREEQYNSTYRVVSVLTVLHQD | |
| Linker in bold | WLNGKEYKCKVSNKALPAPIEKTISKAKGQPREPQVY | |
| IgG sequence | TLPPSRDELTKNQVSLTCLVKGFYPSDIAVEWESNGQ | |
| underlined | PENNYKTTPPVLDSDGSFFLYSKLTVDKSRWQQGNV | |
| | FSCSVMHEALHNHYTQKSLSLSPGK | |
| flexCTLA4-Ig^{LALA} | GCAATGCACGTGGCCCAGCCTGCTGTGGTACTGGC | 53 |
| (nucleic | CAGCAGCCGAGGCATCGCCAGCTTTGTGTGTGAGT | |
| acid sequence) | ATGCATCTCCAGGCAAAGCCACTGAGGTCCGGGTG | |
| | ACAGTGCTTCGGCAGGCTGACAGCCAGGTGACTGA | |
| | AGTCTGTGCGGCAACCTACATGATGGGGAATGAGT | |
| | TGACCTTCCTAGATGATTCCATCTGCACGGGCACCT | |
| | CCAGTGGAAATCAAGTGAACCTCACTATCCAAGGAC | |
| | TGAGGGCCATGGACACGGGACTCTACATCTGCAAG | |
| | GTGGAGCTCATGTACCCACCGCCATACTACCTGGG | |
| | CATAGGCAACGGAACCCAGATTTATGTAATTGGCGA | |
| | TCCAGAACCGTGCCCAGATTCTGACCAGGAGCCCA | |
| | AGAGCAGCGACAAGACCCACACCTGCCCTCCTTGC | |
| | CCTGCTCCAGAGGCTGCTGGCGGCCCTTCCGTGTT | |
| | CCTGTTTCCCCCTAAGCCAAAGGACACCCTGATGAT | |
| | CAGCCGGACCCCAGAGGTGACATGCGTGGTGGTG | |
| | GACGTGTCCCACGAGGACCCCGAGGTGAAGTTCAA | |
| | CTGGTACGTGGATGGCGTGGAGGTGCACAATGCCA | |
| | AGACAAAGCCTCGGGAGGAGCAGTACAACAGCACC | |
| | TATAGAGTGGTGTCCGTGCTGACAGTGCTGCACCA | |
| | GGACTGGCTGAACGGCAAGGAGTACAAGTGCAAGG | |
| | TGTCTAATAAGGCCCTGCCCGCCCCTATCGAGAAG | |
| | ACCATCAGCAAGGCAAAGGGACAGCCAAGGGAGCC | |
| | ACAGGTGTATACACTGCCACCCTCTCGCGACGAGC | |
| | TGACCAAGAACCAAGTGAGCCTGACATGTCTGGTG | |
| | AAGGGCTTCTACCCCTCCGACATCGCCGTGGAGTG | |
| | GGAGTCTAATGGCCAGCCTGAGAACAATTATAAGAC | |
| | CACACCTCCAGTGCTGGACTCCGATGGCTCTTTCTT | |
| | TCTGTACTCTAAGCTGACAGTGGATAAGAGCAGGTG | |
| | GCAGCAGGGCAACGTGTTCAGCTGTAGCGTGATGC | |
| | ACGAAGCCCTGCACAATCACTACACTCAGAAGAGC | |
| | CTGTCCCTGTCACCTGGAAAATGA | |
| -flexCTLA4-Ig^{LALA} | MHVAQPAVVLASSRGIASFVCEYASPGKATEVRVTVL | 54 |
| (amino acid | RQADSQVTEVCAATYMMGNELTFLDDSICTGTSSGN | |
| sequence) | QVNLTIQGLRAMDTGLYICKVELMYPPPYYLGIGNGTQ | |
| CTLA4 | IYVIGDPEPCPDSDQEPKSSDKTHTCPPCPAPEAAGG | |
| sequence in | PSVFLFPPKPKDTLMISRTPEVTCVVVDVSHEDPEVKF | |
| italics | NWYVDGVEVHNAKTKPREEQYNSTYRVVSVLTVLHQ | |
| | DWLNGKEYKCKVSNKALPAPIEKTISKAKGQPREPQV | |
| | YTLPPSRDELTKNQVSLTCLVKGFYPSDIAVEWESNG | |
| | QPENNYKTTPPVLDSDGSFFLYSKLTVDKSRWQQGN | |
| | VFSCSVMHEALHNHYTQKSLSLSPGK | |
| LALA Ig | QEPKSSDKTHTCPPCPAPEAAGGPSVFLFPPKPKDTL | 55 |
| sequence for | MISRTPEVTCVVVDVSHEDPEVKFNWYVDGVEVHNA | |
| use in second | KTKPREEQYNSTYRVVSVLTVLHQDWLNGKEYKCKV | |
| portion, with | SNKALPAPIEKTISKAKGQPREPQVYTLPPSRDELTKN | |
| linker sequence | QVSLTCLVKGFYPSDIAVEWESNGQPENNYKTTPPVL | |
| shown in bold | DSDGSFFLYSKLTVDKSRWQQGNVFSCSVMHEALHN | |
| | HYTQKSLSLSPGK | |
| LALAPG Ig | QEPKSSDKTHTCPPCPAPEAAGGPSVFLFPPKPKDTL | 56 |
| sequence for | MISRTPEVTCVVVDVSHEDPEVKFNWYVDGVEVHNA | |
| use in second | KTKPREEQYNSTYRVVSVLTVLHQDWLNGKEYKCKV | |
| portion, with | SNKALGAPIEKTISKAKGQPREPQVYTLPPSRDELTKN | |
| linker sequence | QVSLTCLVKGFYPSDIAVEWESNGQPENNYKTTPPVL | |
| shown in bold; | DSDGSFFLYSKLTVDKSRWQQGNVFSCSVMHEALHN | |
| LALA mutations | HYTQKSLSLSPGK | |
| shown in bold, | | |
| italics, and ″PG″ | | |
| mutation | | |
| (corresponding | | |
| to P329G) in | | |
| bold, underline | | |
| Flex5 CTLA4- | MHVAQPAVVLASSRGIASFVCEYASPGKATEVRVTVL | 57 |
| Ig^{LALALAPG} | RQADSQVTEVCAATYMMGNELTFLDDSICTGTSSGN | |
| CTLA4 | QVNLTIQGLRAMDTGLYICKVELMYPPPYYLGIGNGTQ | |
| sequence in | IYVIGGGGSDPEPCPDSDQEPKSSDKTHTCPPCPAPE | |
| italics | AAGGPSVFLFPPKPKDTLMISRTPEVTCVVVDVSHED | |
| ″LALA″ | PEVKFNWYVDGVEVHNAKTKPREEQYNSTYRVVSVL | |
| mutations in Ig | TVLHQDWLNGKEYKCKVSNKALGAPIEKTISKAKGQP | |
| portion shown | REPQVYTLPPSRDELTKNQVSLTCLVKGFYPSDIAVE | |
| in bold and | WESNGQPENNYKTTPPVLDSDGSFFLYSKLTVDKSR | |
| ″PG″ mutation | WQQGNVFSCSVMHEALHNHYTQKSLSLSPGK | |
| shown bold | | |
| underlined | | |
| S4d10 CTLA4- | MHVAQPAVVLASSRGIASFVCEYASPGKATEVRVTVL | 58 |
| Ig^{LALAPG} | RQADSQVTEVCAATYMMGNELTFLDDSICTGTSSGN | |
| CTLA4 | QVNLTIQGLRAMDTGLYICKVELMYPPPYYLGIGNGTQ | |
| sequence in | IYVIAKEKKPSYNRGLEPKSSDKTHTCPPCPAPEAAG | |
| italics | GPSVFLFPPKPKDTLMISRTPEVTCVVVDVSHEDPEVK | |
| ″LALA″ | FNWYVDGVEVHNAKTKPREEQYNSTYRVVSVLTVLH | |
| mutations in lg | QDWLNGKEYKCKVSNKALGAPIEKTISKAKGQPREPQ | |
| portion shown | VYTLPPSRDELTKNQVSLTCLVKGFYPSDIAVEWESN | |
| in bold and | GQPENNYKTTPPVLDSDGSFFLYSKLTVDKSRWQQG | |
| ″PG″ mutation | NVFSCSVMHEALHNHYTQKSLSLSPGK | |
| shown bold | | |
| underlined | | |
| S4d10 CTLA4 | MHVAQPAVVLASSRGIASFVCEYASPGKATEVRVTVL | 59 |
| (C terminal | RQADSQVTEVCAATYMMGNELTFLDDSICTGTSSGN | |
| truncated | QVNLTIQGLRAMDTGLYICKVELMYPPPYYLGIGNGTQ | |
| variant of | IYVIAKEKKPSYNRGL | |
| soluble CTLA4 | | |
| sequence | | |
| shown in SEQ | | |
| ID NO: 3; 10 | | |
| amino acid | | |
| truncation of C | | |
| terminus to | | |
| remove | | |
| sequence | | |
| comprising | | |
| cysteine) | | |
| Alternative | EPKSSDKTHTCPPC | 60 |
| linker sequence | | |
| for linking first | | |
| portion with | | |
| second portion | | |
| Alternative | EPKSSDKTHTCPPCPAPEAAGGPSVFLFPPKPKDTLM | 61 |
| LALA Ig | ISRTPEVTCVVVDVSHEDPEVKFNWYVDGVEVHNAKT | |
| sequence for | KPREEQYNSTYRVVSVLTVLHQDWLNGKEYKCKVSN | |
| use in second | KALPAPIEKTISKAKGQPREPQVYTLPPSRDELTKNQV | |
| portion, with | SLTCLVKGFYPSDIAVEWESNGQPENNYKTTPPVLDS | |
| linker sequence | DGSFFLYSKLTVDKSRWQQGNVFSCSVMHEALHNHY | |
| shown in bold | TQKSLSLSPGK | |
| Alternative | EPKSSDKTHTCPPCPAPEAAGGPSVFLFPPKPKDTLM | 62 |
| LALAPG Ig | ISRTPEVTCVVVDVSHEDPEVKFNWYVDGVEVHNAKT | |
| sequence for | KPREEQYNSTYRVVSVLTVLHQDWLNGKEYKCKVSN | |
| use in second | KALGAPIEKTISKAKGQPREPQVYTLPPSRDELTKNQV | |
| portion, with | SLTCLVKGFYPSDIAVEWESNGQPENNYKTTPPVLDS | |
| linker sequence | DGSFFLYSKLTVDKSRWQQGNVFSCSVMHEALHNHY | |
| shown in bold; | TQKSLSLSPGK | |
| LALA mutations | | |
| shown in bold, | | |
| italics, and ″PG″ | | |
| mutation | | |
| (corresponding | | |
| to P329G) in | | |
| bold, underline | | |

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

The general chemical terms used in the formulae herein have their usual meaning.

### The CTLA4/B7 Axis

The CTLA4 immune checkpoint restrains T cell activity and prevents autoimmunity. T cell surface CTLA4 inhibits CD28 receptor costimulatory signaling by sequestering their shared ligands CD80 and CD86 (B7-1 and B7-2, respectively) expressed on antigen-presenting cells (APCs). Similarly, recombinant CTLA4-Ig fusion proteins including abatacept block costimulation and are effective therapies in autoimmune disease and organ transplantation.

Recent studies show that CD80 and the coinhibitory ligand PDL1 interact in cis on the same APC surface, but how this affects CTLA4 and PD1 checkpoint function is poorly understood.

Here the inventors have surprisingly shown that upon binding to their cognate ligands, existing therapeutics acting as CTLA4 agonists (such as abatacept) disrupt surface cis-CD80:PDL1 complexes, promoting PDL1 engagement of neighbouring cell surface PD1. Transmembrane CTLA4 similarly releases PDL1 from cis-bound CD80 on interacting cells, reinforcing a PDL1-replete inhibitory state upon CTLA4-induced CD80 transendocytosis.

In contrast, the inventors demonstrate that monomeric CTLA4 binds CD80 without liberating PDL1. The inventors have therefore developed a series of homodimeric CTLA4-Ig variants that maintain bivalent CD80 binding without displacing, or only partly displacing, cis-bound PDL1. Surprisingly, the inventors found that rather than reducing the immunosuppressive action that normally results from CD80/CTLA4 binding, administration of the CTLA-Ig variants of the invention resulted in immunostimulation in accepted animal models of inflammatory disease. These findings indicate that CTLA4-Ig variants that fail to liberate PDL1 from cis-bound CD80, or only partially liberate PDL1 from cis-bound CD80, may block the PDL1-liberating functions of endogenous CTLA4, resulting in a net immunostimulatory effect.

### First Portion of the Fusion Protein

The fusion proteins of the present invention comprise a first portion comprising or consisting of the amino acid sequence of a CTLA4 protein, or functional variant thereof (comprising a CTLA4-ligand binding domain, or having a sequence corresponding to the transmembrane form of CTLA4 or the soluble isoform of CTLA4).

In the context of the first or second aspects of the invention, the fusion protein may comprise a variant extracellular domain of a transmembrane isoform of CTLA4. In the context of the third aspect of the invention, the fusion protein comprises the sequence of a soluble isoform of CTLA4. The sequence of the soluble isoform may be a variant sequence or it may be a “wild-type” sequence.

However, it will generally be appreciated that the fusion proteins of the invention may comprise any sequence derived from a CTLA4 protein, whether the transmembrane form or soluble protein, provided that the sequence comprises amino acid residues for enabling binding to CD80. Typically, the fusion proteins of the invention will comprise at least the sequence at set forth in SEQ ID NO: 1, or a sequence at least 80%, 81%, 82%, 83%, 84%, 85%, 86%, 87%, 88%, 89%, 90%, 91%, 92%, 93%, 94%, 95%, 96%, 97%, 98%, or at least 99% identical thereto.

In any aspect of the invention, and preferably in the context of the second aspect of the invention, the first portion of the fusion protein comprises an insertion, substitution or deletion of amino acid residues in the C-terminal region of the portion (corresponding to the dimerisation interface of the TM form of CTLA4). This region corresponds to residues 100 to 124 of SEQ ID NO: 2, (or region equivalent thereto), wherein SEQ ID NO: 2 corresponds to the amino acid sequence of the extracellular domain of a transmembrane isoform of CTLA4. The insertion, substitution or deletion may be of any one or more amino acid sequences.

The insertion, substitution or deletion is preferably located in the C-terminal region of the CTLA4 sequence, preferably within the last 20 amino acids of an extracellular domain of CTLA4. More than one insertion, substitution or deletion may be included, and indeed any arrangement may be contemplated. Preferably there is an insertion of at least one amino acid residue in the region 20 amino acids N-terminal to the cysteine at residue 120 of SEQ ID NO: 2 or a substitution of at least one amino acid residue in this region (or equivalent thereto), provided that the sequence N-terminal to the cysteine residue provides for destabilisation of the homodimer interface of the CTLA4 protein, compared to a protein having the sequence of SEQ ID NO: 2.

In certain embodiments, the insertion is at or C-terminal to the cysteine at residue 120 of SEQ ID NO: 2, or is a substitution of at least one amino acid in this region. In such embodiments, dimerisation may be further provided by residues in the second portion (i.e., the Fc region of an antibody).

In any embodiment, variation comprises an insertion, substitution or deletion of one or more amino acid residues compared to the sequence of SEQ ID NO: 1 or 2. In the case of an insertion, typically, the insertion comprises between about 1 to about 30 amino acid residues, optionally wherein the insertion comprises 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues at a position that is between residues 100 to 124 of SEQ ID NO: 2 or between residues 100 to 115 of SEQ ID NO: 1, or position equivalent thereto.

The substitution may be a substitution of one or more amino acid residues. Typically, the number of amino acid substitutions comprises between about 1 to about 30 amino acid residues, optionally wherein the number of amino acid substitutions comprise substitutions of about 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid residues, compared to the sequence of SEQ ID NO: 1 or 2 in the region between residues 100 to 124 of SEQ ID NO: 2 or between residues 100 to 115 of SEQ ID NO: 1, or position(s) equivalent thereto.

The deletion may be deletion of one or more amino acid residues. Typically, the number of amino acid deletions comprises between about 1 to about 30 amino acid residues, preferably no more than a deletion of about 1, 2, 3, 4, or 5 amino acids in the region between residues 100 to 124 of SEQ ID NO: 2, or position(s) equivalent thereto.

In certain embodiments, more than one insertion, substitution or deletion may be included in the C-terminal region of the CTLA4, or region between residues 100 to 124 SEQ ID NO: 2, or region equivalent thereto. Indeed, any arrangement may be contemplated provided that the amino acid residues in the C-terminal region of SEQ ID NO: 2, or of the CTLA4, provide for flexibility in this region of the protein compared to a protein having the sequence of SEQ ID NO: 2.

In certain preferred embodiments, the insertion, deletion or substitution is N-terminal to the cysteine residue located at residue 120 of SEQ ID NO: 2 (or equivalent position). Preferably, there is an insertion, substitution or deletion of at least one amino acid residue in the region 20 amino acids N-terminal to the cysteine at residue 120 of SEQ ID NO: 2. In particularly preferred embodiments, insertion, substitution or deletion causes destabilisation of the homodimer interface N-terminal of the cysteine residue located at residue 120 of SEQ ID NO: 2.

In certain preferred embodiments, an insertion, substitution or deletion is between, or is between residues equivalent to residues 100 and 101; between residues 101 and 102; between residues 102 and 103; between residues 103 and 104; between residues 104 and 105; between residues 105 and 106; between residues 106 and 107; between residues 107 and 108; between residues 108 and 109; between residues 109 and 110; between residues 110 and 111; between residues 111 and 112; between residues 112 and 113; between residues 113 and 114; between residues 114 and 115; between residues 115 and 116; between residues 116 and 117; between residues 117 and 118; between residues 118 and 119; between residues 119 and 120 of SEQ ID NO: 2 between residues 120 and 121 of SEQ ID NO: 1, between residues 121 and 122 of SEQ ID NO: 2, between residues 122 and 123 of SEQ ID NO: 1 or 2, or between residues 123 and 124 of SEQ ID NO: 2, or residues equivalent to those residues defined in SEQ ID NO: 2.

In certain preferred embodiments, the insertion is inserted between residues 115 and 116 of SEQ ID NO: 2.

In alternative embodiments, the amino acid substitution may be substitution of a region comprising any one of residues 110, 111, 112, 113, 114, 115, 116, 117, 118, 119, 120, 121, 122, 123 or 124 of SEQ ID NO: 2; or may preferably comprise residues 118 and 119 of SEQ ID NO: 2, residues 117 to 119 of SEQ ID NO: 2, residues 116 to 119 of SEQ ID NO: 2, residues 115 to 119 of SEQ ID NO: 2, residues 114 to 119 of SEQ ID NO: 2, residues 113 to 119 of SEQ ID NO: 2, residues 112 to 119 of SEQ ID NO: 2, residues 111 to 119 of SEQ ID NO: 2, residues 110 to 119 of SEQ ID NO: 2, or of regions equivalent thereto.

In a preferred embodiment, the amino acid substitution is of the region comprising residues 116 to 119 of SEQ ID NO: 2 (or region equivalent thereto).

In any embodiment, and regardless of the location of the insertion, substitution or deletion, the insertion, substitution or deletion may be at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 or more amino acids.

It will be appreciated that any amino acid or combination of amino acids may be inserted, or included in the flexible domain (e.g., by substitution of the amino acid residues defined in SEQ ID NO: 1 or 2). In any embodiment, the insertion or flexible domain is comprised of amino acid residues selected from glycine, serine, alanine residues.

Similarly, in the context of an amino acid substitution, the amino acids that are substituted preferably comprise any amino acids that facilitate formation of the flexible domain and/or result in destabilisation of the region of the protein N-terminal to the cysteine residue at position 120 of SEQ ID NO: 2 (or position equivalent thereto). Examples of suitable amino acid residues for substitution include glycine, serine, alanine, lysine and glutamic acid, preferably glycine or serine.

As used herein, a CTLA4 molecule or a CTLA4 domain comprises a CD28-binding domain of CTLA4A. Typically, the sequence is derived from a wild-type CTLA4 molecule, although in certain embodiments, the sequence may be from a mutant CTLA4 molecule or a fragment thereof or a portion thereof. The mutant CTLA4 molecule may have increased binding affinity for B7-1.

As used herein and in the context of the first aspect of the invention, “wild type CTLA4” has the amino acid sequence of naturally occurring, full length transmembrane isoform of CTLA4 (U.S. Pat. Nos. 5,434,131, 5,844,095, 5,851,795), or the extracellular domain thereof, which binds CD80 and/or CD86, and/or interferes with CD80 and/or CD86 from binding their receptors.

Exemplary amino acid sequences of mature, wild type CTLA4 sequence are provided in Table 1 as SEQ ID NOs: 2 and 3, with the ligand binding sequence shown in SEQ ID NO: 1. (The N-terminal secretion peptide is not shown in this sequence). It will be appreciated that wild-type CTLA4 sequences may comprise alternative N-terminal leader sequences which may alter the N-terminal sequence relative to SEQ ID NO: 1, 2 or 3. Such variants are included within the scope of the present invention. In one example, the sequence of the wild type CTLA4 sequence may be as described in the NCBI database at accession number NP_005205.2. In the context of that accession entry, the cysteine in the C-terminal region is located at position 122 of the sequence (rather than position 120 as is the case in SEQ ID NO: 1 or 2). It will be appreciated, therefore, that any reference to a position or residue “equivalent thereto” as described herein, is to be interpreted as defining an equivalent amino acid residue which is defined as being at a different position by virtue of a different accession sequence.

As used herein an amino acid residue at the position equivalent to any position in SEQ ID NO: 1 (or SEQ ID NO: 2 or 3) can be determined by any means known to a person skilled in the art. For example, an alignment of one or more sequences with an amino acid sequence of SEQ ID NO: 1, 2 or 3 would allow a person skilled in the art to determine the amino acid at the position equivalent to position in SEQ ID NO: 1, 2 or 3. A person skilled in the art can compare the three dimensional structure of a polypeptide with the three dimensional structure of a polypeptide having the amino acid sequence of SEQ ID NO: 1 and determine the amino acid residue that is at an equivalent position to that in SEQ ID NO: 1.

In particular embodiments, the extracellular domain of wild type CTLA4 begins with methionine at position +1 and ends at aspartic acid at position +124, or the extracellular domain of wild type CTLA4 begins with alanine at position-1 and ends at aspartic acid at position +124.

Wild type CTLA4 is a cell surface protein, having an N-terminal extracellular domain, a transmembrane domain, and a C-terminal cytoplasmic domain. The extracellular domain binds to target antigens, such as CD80 and CD86. In a cell, the naturally occurring, wild type CTLA4 protein is translated as an immature polypeptide, which includes a signal peptide at the N-terminal end. The immature polypeptide undergoes post-translational processing, which includes cleavage and removal of the signal peptide to generate a CTLA4 cleavage product having a newly generated N-terminal end that differs from the N-terminal end in the immature form. One skilled in the art will appreciate that additional post-translational processing may occur, which removes one or more of the amino acids from the newly generated N-terminal end of the CTLA4 cleavage product. The mature form of the CTLA4 molecule includes the extracellular domain of CTLA4, or any portion thereof, which binds to CD80 and/or CD86.

As used herein “the extracellular domain of CTLA4” is a portion of the CTLA4 that recognizes and binds CD80 and/or CD86. For example, an extracellular domain of CTLA4 comprises methionine at position 1 to aspartic acid at position +124. Alternatively, an extracellular domain of CTLA4 comprises alanine at position-1 to aspartic acid at position +124. The extracellular domain includes fragments or derivatives of CTLA4 that bind CD80 and/or CD86.

As used herein “a CTLA4 mutant molecule” comprises any variant of a CTLA4 molecule (such as any variant of the amino acid sequence set forth in SEQ ID NO: 1 or 2), wherein the molecule recognizes and binds its target, e.g., CD80 and/or CD86.

As used herein a “fragment of a CTLA4 mutant molecule” is a part of a CTLA4 mutant molecule, preferably the extracellular domain of CTLA4 or a part thereof, that recognizes and binds its target, e.g., CD80 and/or CD86.

As used herein a “derivative of a CTLA4 mutant molecule” is a molecule that shares at least 70% sequence similarity with and functions like the extracellular domain of CTLA4, i.e., it recognizes and binds CD80 and/or CD86.

As used herein, a “portion of a CTLA4 molecule” includes fragments and derivatives of a CTLA4 molecule that binds CD80 and/or CD86.

As used herein “a position equivalent thereto” refers to an amino acid residue that is at an equivalent position within the CTLA4 protein but may be numbered according to a different numbering convention. The skilled person will be able to determine which amino acid position is being referred to by the context of the surrounding amino acid sequences. For example, the arginine residue in the context of the amino acid sequence ATEVRVTVL is numbered residue R33 according to the numbering of SEQ ID NO: 2, but may be referred to differently according to a different numbering convention.

Included within the scope of the present invention, are fusion proteins wherein the first portion comprises additional variations to the CTLA4 sequence, for example, to improve binding affinity for the ligands of CTLA4. In certain examples, the first portion further includes one or more of the substitutions selected from the group consisting of A29E, A29F, A29H, A29K, A29N, A29Q, A29R, T30E, T30H, T30R, T30V, E31D, E311, E31M, E31T, E31V, R33E, R33F, R331, R33L, R33M, R33Q, R33T, R33W, R33Y, T35D, T35E, T35F, T35M, T35V, T35Y, A49D, A49E, A49F, A49T, A49W, A49Y, T51D, T51E, T51H, T51L, T51N, T51Q, T51R, T51S, T51V, M53E, M53F, M53H, M53Q, M53W, M53Y, T59H, T591, T59L, T59N, T59Q, T59V, T59Y, L61A, L61D, L61E, L61F, L61G, L61H, L61, L61K, L61M, L61N, L61P, L61Q, L61R, L61S, L61T, L61V, L61W, L61Y, D63E, S64K, S64R, S64Y, K93D, K93E, K93F, K93H, K93N, K93Q, K93R, K93S, K93T, K93V, K93W, K93Y, E95D, E95H, E95L, E95Q, E95Y, M97D, M97F, M971, M97N, M97V, Y98F, Y98W, Y102F, Y102W, Y103D, Y103E, Y103F, Y103H, Y103N, Y103Q, Y103W, L104F, L104H, L104M, L104V, L104Y, G105D, G105E, 1106E, and 1106Y (numbering according to SEQ ID NO: 2; it will be well within the purview of the skilled person to determine the particular amino acid substitutions listed above, if comparing to an alternative numbering convention for CTLA4. For example, the “Y100F” substitution sometimes referred to in the literature is numbered Y98F according to the numbering of any of SEQ ID NO: 1 or 2 or 3.). Other variants contemplated by the present invention include 116R, A24T, S25N, G27S, L58A, S70A and M85Q (numbered according to any of SEQ ID NOs: 1 to 3).

In accordance with the third aspect of the invention, the first comprises the sequence of a soluble isoform of CTLA4. An exemplary amino acid sequence of mature, soluble CTLA4 is provided in Table 1 as SEQ ID NO: 3.

### Second Portion: Fc Region of an Antibody

The Fc portion of the fusion proteins of the invention are comprised of the Fc region or some portion of the Fc region of an antibody. Antibodies are immunoglobulins that bind a specific antigen. In most mammals, including humans and mice, antibodies are constructed from paired heavy and light polypeptide chains. The light and heavy chain variable regions show significant sequence diversity between antibodies, and are responsible for binding the target antigen. Each chain is made up of individual immunoglobulin (Ig) domains, and thus the generic term immunoglobulin is used for such proteins.

The term “Fc region” herein is used to define a C-terminal region of an immunoglobulin heavy chain that contains at least a portion of the constant region. In other words, the Fc region contains two heavy chain fragments comprising the CH2 and CH3 domains of an antibody. In the context of the present invention, the Fc region comprises two heavy chain fragments, preferably the CH2 and CH3 domains of said heavy chain. The two heavy chain fragments are held together by two or more disulfide bonds and by hydrophobic interactions of the CH3 domains.

In some aspects, the fusion protein does not exhibit any effector function or any detectable effector function. “Effector functions” or “effector activities” refer to those biological activities attributable to the Fc region of an antibody, which vary with the antibody isotype. Examples of antibody effector functions include: Clq binding and complement dependent cytotoxicity (CDC); Fc receptor binding; antibody dependent cell-mediated cytotoxicity (ADCC); phagocytosis; down regulation of cell surface receptors (e.g. B cell receptor); and B cell activation. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). Clq binding assays may also be carried out to confirm that the antibody is unable to bind Clq and hence lacks CDC activity. See, e.g., Clq and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18 (12): 1759-1769 (2006); WO 2013/120929 AI).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). For example, an antibody variant may comprise an Fc region with one or more amino acid substitutions which diminish FcγR binding, e.g., substitutions at positions 234 and 235 of the Fc region (EU numbering of residues). For example, the substitutions are L234A and L235A (LALA) (See, e.g., WO 2012/130831); (eg SEQ ID NO: 55 or 61). The substitutions may additionally include substitution of the proline residue at position 329, such as a P329G mutation to disable binding to FcR (eg SEQ ID NO: 56 or 62). Further, alterations may be made in the Fc region that result in altered (i.e., diminished) Clq binding and/or Complement Dependent Cytotoxicity (CDC), e.g., as described in U.S. Pat. No. 6,194,551, WO 99/51642, and Idusogie et al. J. Immunol. 164:4178-4184 (2000).

In some aspects, the Fc region includes mutations to the complement (Clq) and/or to Fc gamma receptor (FcγR) binding sites. In some aspects, such mutations can render the fusion protein incapable of antibody directed cytotoxicity (ADCC) and complement directed cytotoxicity (CDC).

The Fc region as used in the context of the present invention does not trigger cytotoxicity such as antibody-dependent cellular cytotoxicity (ADCC) or complement dependent cytotoxicity (CDC).

The term “Fc region” also includes native sequence Fc regions and variant Fc regions. The Fc region may include the carboxyl-terminus of the heavy chain. Antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991. Amino acid sequence variants of the Fc region of an antibody may be contemplated. Amino acid sequence variants of an Fc region of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the Fc region of the antibody. Any combination of deletion, insertion, and substitution can be made to arrive at the final construct, provided that the final construct possesses the desired characteristics, e.g., inducing or supporting an immunostimulatory response.

The Fc region of the antibody may be an Fc region of any of the classes of antibody, such as IgA, IgD, IgE, IgG, and IgM. The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. Accordingly, as used in the context of the present invention, the antibody may be an Fc region of an IgG. For example, the Fc region of the antibody may be an Fc region of an IgG1, an IgG2, an IgG2b, an IgG3 or an IgG4. In some aspects, the fusion protein of the present invention comprises an IgG of an Fc region of an antibody. In the context of the present invention, the Fc region of the antibody is an Fc region of an IgG, preferably IgG1.

In preferred embodiments, the fusion proteins disclosed herein may incorporate Fc variants that improve FcRn binding. Such variants may enhance the in vivo pharmacokinetic properties of the CTLA4-Ig fusions. Preferred variants that increase binding to FcRn and/or improve pharmacokinetic properties include but are not limited to substitutions at positions 259, 308, 428, and 434, including but not limited to for example 2591, 308F, 428L, 428M, 434S, 434H, 434F, 434Y, and 434M (U.S. Ser. No. 12/341,769, filed Dec. 22, 2008, entitled “Fc Variants with Altered Binding to FcRn”, entirely incorporated by reference). Other variants that increase Fc binding to FcRn include but are not limited to: 250E, 250Q, 428L, 428F, 250Q/428L (Hinton et al., 2004, J. Biol. Chem. 279 (8): 6213-6216, Hinton et al. 2006 Journal of Immunology 176:346-356), 256A, 272A, 286A, 305A, 307A, 307Q, 311A, 312A, 376A, 378Q, 380A, 382A, 434A (Shields et al, Journal of Biological Chemistry, 2001, 276 (9): 6591-6604, entirely incorporated by reference), 252F, 252T, 252Y, 252W, 254T, 256S, 256R, 256Q, 256E, 256D, 256T, 309P, 311S, 433R, 433S, 433I, 433P, 433Q, 434H, 434F, 434Y, 252Y/254T/256E, 433K/434F/436H, 308T/309P/311S (dall Acqua et al. Journal of Immunology, 2002, 169:5171-5180, Dall'Acqua et al., 2006, Journal of Biological Chemistry 281:23514-23524, entirely incorporated by reference). Other modifications for modulating FcRn binding are described in Yeung et al., 2010, J Immunol, 182:7663-7671.

Other Fc modifications for use in the present invention include variants that reduce or ablate binding to FcγRs and/or complement proteins, thereby reducing or ablating Fc-mediated effector functions such as ADCC, ADCP, and CDC. Such variants are also referred to herein as “knockout variants” or “KO variants”. Variants that reduce binding to FcγRs and complement are useful for reducing unwanted interactions mediated by the Fc region and for tuning the selectivity of the CTLA4-Ig fusion proteins. Preferred knockout variants are described in US 2008-0242845 A1, published on Oct. 2, 2008, entitled “Fc Variants with Optimized Properties, expressly incorporated by reference herein. Preferred modifications include but are not limited substitutions, insertions, and deletions at positions 234, 235, 236, 237, 267, 269, 325, and 328, wherein numbering is according to the EU index. Preferred substitutions include but are not limited to 234G, 235G, 236R, 237K, 267R, 269R, 325L, and 328R, wherein numbering is according to the EU index. A preferred variant comprises 236R/328R. Variants may be used in the context of any IgG isotype or IgG isotype Fc region, including but not limited to human IgG1, IgG2, IgG3, and/or IgG4. Preferred IgG Fc regions for reducing FcγR and complement binding and reducing Fc-mediated effector functions are IgG2 and IgG4 Fc regions. Hybrid isotypes may also be useful, for example hybrid IgG1/IgG2 isotypes as described in U.S. Ser. No. 11/256,060. Other modifications for reducing FcγR and complement interactions include but are not limited to substitutions 297A, 234A, 235A, 237A, 318A, 228P, 236E, 268Q, 309L, 330S, 331S, 220S, 226S, 229S, 238S, 233P, and 234V, as well as removal of the glycosylation at position 297 by mutational or enzymatic means or by production in organisms such as bacteria that do not glycosylate proteins. These and other modifications are reviewed in Strohl, 2009, Current Opinion in Biotechnology 20:685-691, incorporated by reference in its entirety.

In certain embodiments, particularly in accordance with the second aspect of the invention, it is desirable to utilise Fc regions of antibodies retain the ability to homodimerise. Such Fc sequences will be known to the skilled person. For example, in the interface of two IgG1 CH3 domains, there are at least 16 residues in each chain contributing to hydrophobic interactions (eg L351, T366, T/L368, P395, F405, Y407, and K409) involved in homodimeric interactions.

In alternative embodiments, Fc modifications that improve binding to FcγRs and/or complement may also find use in the CTLA4-Ig fusions herein. Such Fc variants may enhance Fc-mediated effector functions such as ADCC, ADCP, and/or CDC. Preferred modifications for improving FcγR and complement binding are described in US 2006-0024298 A1, published on Feb. 2, 2006, and US 2006-0235208 A1, published on Oct. 19, 2006, expressly incorporated herein by reference. Preferred modifications comprise a substitution at a position selected from the group consisting of 236, 239, 268, 324, and 332, wherein numbering is according to the EU index. Preferred substitutions include but are not limited to 236A, 239D, 239E, 268D, 267E, 268E, 268F, 324T, 332D, and 332E. Preferred variants include but are not limited to 239D/332E, 236A/332E, 236A/239D/332E, 268F/324T, 267E/268F, 267E/324T, and 267E/268F/324T. Other modifications for enhancing FcγR and complement interactions include but are not limited to substitutions 298A, 333A, 334A, 326A, 2471, 339D, 339Q, 280H, 290S, 298D, 298V, 243L, 292P, 300L, 396L, 3051, and 396L. These and other modifications are reviewed in Strohl, 2009, ibid.

In one embodiment, the CTLA4-Ig fusions disclosed herein may incorporate Fc variants that enhance affinity for an inhibitory receptor FcγRIIb. Such variants may provide the CTLA4-Ig fusions herein with immunomodulatory activities related to FcγRIIb+ cells, including for example B cells and monocytes. In one embodiment, the Fc variants provide selectively enhanced affinity to FcγRIIb relative to one or more activating receptors. Modifications for altering binding to FcγRIIb are described in U.S. Ser. No. 12/156,183, filed May 30, 2008, entitled “Methods and Compositions for Inhibiting CD32b Expressing Cells”, herein expressly incorporated by reference. In particular, Fc variants that improve binding to FcγRIIb may include one or more modifications at a position selected from the group consisting of 234, 235, 236, 237, 239, 266, 267, 268, 325, 326, 327, 328, and 332, according to the EU index. Preferable substitutions for enhancing FcγRIIb affinity include but are not limited to 234D, 234E, 234W, 235D, 235F, 235R, 235Y, 236D, 236N, 237D, 237N, 239D, 239E, 266M, 267D, 267E, 268D, 268E, 327D, 327E, 328F, 328W, 328Y, and 332E. More preferably, substitutions include but are not limited to 235Y, 236D, 239D, 266M, 267E, 268D, 268E, 328F, 328W, and 328Y. Preferred Fc variants for enhancing binding to FcγRIIb include but are not limited to 235Y/267E, 236D/267E, 239D/268D, 239D/267E, 267E/268D, 267E/268E, and 267E/328F.

CTLA4-Ig fusion proteins described herein can incorporate Fc modifications in the context of any IgG isotype or IgG isotype Fc region, including but not limited to human IgG1, IgG2, IgG3, and/or IgG4. The IgG isotype may be selected such as to alter FcγR- and/or complement-mediated effector function(s). Hybrid IgG isotypes may also be useful. For example, U.S. Ser. No. 11/256,060 describes a number of hybrid IgG1/IgG2 constant regions that may find use in the particular invention. In some embodiments of the invention, CTLA4-Ig fusion proteins may comprise means for isotypic modifications, that is, modifications in a parent IgG to the amino acid type in an alternate IgG. For example, an IgG1/IgG3 hybrid variant may be constructed by a substitutional means for substituting IgG1 positions in the CH2 and/or CH3 region with the amino acids from IgG3 at positions where the two isotypes differ. Thus, a hybrid variant IgG antibody may be constructed that comprises one or more substitutional means, e.g., 274Q, 276K, 300F, 339T, 356E, 358M, 384S, 392N, 397M, 422I, 435R, and 436F. In other embodiments of the invention, an IgG1/IgG2 hybrid variant may be constructed by a substitutional means for substituting IgG2 positions in the CH2 and/or CH3 region with amino acids from IgG1 at positions where the two isotypes differ. Thus, a hybrid variant IgG antibody may be constructed that comprises one or more substitutional means, e.g., one or more of the following amino acid substitutions: 233E, 234L, 235L,-236G (referring to an insertion of a glycine at position 236), and 327A.

### Linkers

CTLA4 variants may be linked to Fc regions via a linker. The term “linker” is used to denote polypeptides comprising two or more amino acid residues joined by peptide bonds and are used to link one or more antigen binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). A variety of linkers may find use in some embodiments described herein to covalently link Fc regions to a fusion partner.

“Linker” herein is also referred to as “linker sequence”, “spacer”, “tethering sequence” or grammatical equivalents thereof. Homo- or hetero-bifunctional linkers as are well known (see, 1994 Pierce Chemical Company catalog, technical section on cross-linkers, pages 155-200, incorporated entirely by reference). A number of strategies may be used to covalently link molecules together. These include, but are not limited to polypeptide linkages between N- and C-termini of proteins or protein domains, linkage via disulfide bonds, and linkage via chemical cross-linking reagents. In one aspect of this embodiment, the linker is a peptide bond, generated by recombinant techniques or peptide synthesis. The linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used. Useful linkers include glycine-serine polymers, including for example (GS)n, (GSGGS)n, (GGGGS)n, and (GGGS)n, where n is an integer of at least one, glycine-alanine polymers, alanine-serine polymers, and other flexible linkers. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers, that is may find use as linkers The fusion proteins of the invention may comprise a linker region (or spacer) located between the first and second portions.

In the context of the present invention, the polypeptide comprising or consisting of the amino acid sequence of a CTLA4 variant is fused via a linker at the C-terminus to the Fc region or Fc receptor binding domain.

A linker is usually a peptide having a length of up to 20 amino acids. The term “linked to” or “fused to” refers to a covalent bond, e.g., a peptide bond, formed between two moieties. Accordingly, in the context of the present invention the linker may have a length of 1, 2, 3, 4, 5, 6, 7, 8, 9, 10, 11, 12, 13, 14, 15, 16, 17, 18, 19, 20, 21 or 22 amino acids. For example, the herein provided fusion protein may comprise a linker between the polypeptide comprising or consisting of an amino acid sequence of a CLTA4 variant and the Fc region of the antibody, such as between the N-terminus of the Fc regions/FcR binding domains and the C-terminus of the polypeptide. Such linkers have the advantage that they can make it more likely that the different polypeptides of the fusion protein fold independently and behave as expected.

Thus, in the context of the present invention the polypeptide comprising or consisting of the first and second portions may be comprised in a single-chain multi-functional polypeptide.

In some aspects, the fusion protein of the present invention includes a peptide linker. The skilled person will be familiar with the design and use of various peptide linkers comprised of various amino acids, and of various lengths, which would be suitable for use as linkers in accordance with the present invention. The linker may comprise various combinations of repeated amino acid sequences.

The linker may be a flexible linker (such as those comprising repeats of glycine and serine residues), a rigid linker (such as those comprising glutamic acid and lysine residues, flanking alanine repeats) and/or a cleavable linker (such as sequences that are susceptible by protease cleavage). Examples of such linkers are known to the skilled person and are described for example, in Chen et al., (2013) Advanced Drug Delivery Reviews, 65:1357-1369.

In some aspects, the peptide linker may include the amino acids glycine and serine in various lengths and combinations. In some aspects, the peptide linker can include the sequence Gly-Gly-Ser (GGS), Gly-Gly-Gly-Ser (GGGS) or Gly-Gly-Gly-Gly-Ser (GGGGS) and variations or repeats thereof. In some aspects, the peptide linker can include the amino acid sequence GGGGS (a linker of 6 amino acids in length) or even longer. The linker may a series of repeating glycine and serine residues (GS) of different lengths, i.e., (GS)n where n is any number from 1 to 15 or more. For example, the linker may be (GS)3 (i.e., GSGSGS) or longer (GS)11 or longer. It will be appreciated that n can be any number including 1, 2, 3, 4, 5, 6, 7, 8, 9, 10, 11 or more. Fusion proteins having linkers of such length are included within the scope of the present invention. Similarly, the linker may be a series of repeating glycine residues separated by serine residues. For example (GGGGS)3 (i.e., the linker may comprise the amino acid sequence GGGGSGGGGSGGGGS (G4S)3.) and variations thereof.

The peptide linker may consist of a series of repeats of Thr-Pro (TP) comprising one or more additional amino acids N and C terminal to the repeat sequence. For example, the linker may comprise or consist of the sequence GTPTPTPTPTGEF (also known as the TP5 linker). In further aspects, the linker may be a short and/or alpha-helical rigid linker (e.g. A (EAAAK) 3A, PAPAP or a dipeptide such as LE).

In certain aspects, the linker may be flexible and cleavable. Such linkers preferably comprise one or more recognition sites for a protease to enable cleavage.

Preferred linkers of the invention comprise sequences from an antibody hinge region. Hinge regions sequences from any antibody isotype may be used, including for example hinge sequences from IgG1, IgG2, IgG3, and/or IgG4. Linker sequences may also include any sequence of any length of CL/CH1 domain but not all residues of CL/CH1 domain; for example, the first 5-12 amino acid residues of the CL/CH1 domains. Linkers can be derived from immunoglobulin heavy chains of any isotype, including for example Cγ1, Cγ2, Cγ3, Cγ4, Cα1, Cα2, Cδ, Cε, and Cμ. Linkers can be derived from immunoglobulin light chain, for example Cκ or Cλ. Linker sequences may also be derived from other proteins such as Ig-like proteins (e.g. TCR, FcR, KIR), hinge region-derived sequences, and other natural sequences from other proteins. Examples of such suitable linker sequences are provided herein in Table 1, non-limiting examples of which are defined in SEQ ID NOs: 26 to 34 and 60.

### Nucleic Acids

The present invention provides various nucleic acid constructs, or isolated nucleic acids encoding the fusion proteins described herein.

An “isolated” nucleic acid molecule is a nucleic acid molecule that is identified and separated from at least one contaminant nucleic acid molecule with which it is ordinarily associated in the natural source. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes nucleic acid molecules contained in cells that ordinarily express, for example, CTLA4, where, for example, the nucleic acid molecule is in a chromosomal location different from that of natural cells.

The terms “nucleic acid molecule” and “polynucleotide” are used interchangeably herein and refer to a polymeric form of nucleotides of any length, either deoxyribonucleotides or ribonucleotides, or analogs thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or purified form. A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at 5′ (amino) terminus and a translation stop codon at 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

Polynucleotides of the invention can be synthesised according to methods well known in the art, as described by way of example in Sambrook et al (1989, Molecular Cloning-a laboratory manual; Cold Spring Harbor Press).

The polynucleotide molecules of the present invention may be provided in the form of an expression cassette which includes control sequences operably linked to the inserted sequence, thus allowing for expression of the polypeptide of the invention in vivo in a targeted subject. These expression cassettes, in turn, are typically provided within vectors (e.g., plasmids or recombinant viral vectors) which are suitable for use as reagents for nucleic acid immunization. Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

The present invention thus includes expression vectors that comprise such polynucleotide sequences.

Furthermore, it will be appreciated that the compositions and products of the invention may comprise a mixture of polypeptides and polynucleotides. Accordingly, the invention provides a composition or product as defined herein, wherein in place of any one of the polypeptides is a polynucleotide capable of expressing said polypeptide.

Expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art.

Thus, the methods of the present invention include delivering such a vector to a cell and allowing transcription from the vector to occur. Preferably, a polynucleotide of the invention or for use in the invention in a vector is operably linked to a control sequence which is capable of providing for the expression of the coding sequence by the host cell, i.e. the vector is an expression vector.

“Operably linked” refers to an arrangement of elements wherein the components so described are configured so as to perform their usual function. Thus, a given regulatory sequence, such as a promoter, operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence.

A number of expression systems have been described in the art, each of which typically consists of a vector containing a gene or nucleotide sequence of interest operably linked to expression control sequences. These control sequences include transcriptional promoter sequences and transcriptional start and termination sequences. The vectors of the invention may be for example, plasmid, virus or phage vectors provided with an origin of replication, optionally a promoter for the expression of the said polynucleotide and optionally a regulator of the promoter. A “plasmid” is a vector in the form of an extra-chromosomal genetic element. The vectors may contain one or more selectable marker genes, for example an ampicillin resistance gene in the case of a bacterial plasmid or a resistance gene for a fungal vector. Vectors may be used in vitro, for example for the production of DNA or RNA or used to transfect or transform a host cell, for example, a mammalian host cell. The vectors may also be adapted to be used in vivo, for example to allow in vivo expression of the polypeptide.

A “promoter” is a nucleotide sequence which initiates and regulates transcription of a polypeptide-encoding polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

Exemplary promoters active in mammalian cells include cytomegalovirus immediate early promoter (CMV-IE), human elongation factor 1-α promoter (EF1), small nuclear RNA promoters (U1a and U1b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

A polynucleotide, expression cassette or vector according to the present invention may additionally comprise a signal peptide sequence. The signal peptide sequence is generally inserted in operable linkage with the promoter such that the signal peptide is expressed and facilitates secretion of a polypeptide encoded by coding sequence also in operable linkage with the promoter.

Typically, a signal peptide sequence encodes a peptide of 10 to 30 amino acids for example 15 to 20 amino acids. Often the amino acids are predominantly hydrophobic. In a typical situation, a signal peptide targets a growing polypeptide chain bearing the signal peptide to the endoplasmic reticulum of the expressing cell. The signal peptide is cleaved off in the endoplasmic reticulum, allowing for secretion of the polypeptide via the Golgi apparatus. Thus, a peptide of the invention may be provided to an individual by expression from cells within the individual, and secretion from those cells.

In any embodiment of the invention, a polypeptide or protein sequence described herein may be expressed as a polypeptide that comprises a signal peptide sequence, such as the sequence MGVLLTQRTLLSLVLALLFPSMASMA, or a sequence at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% identical thereto. It will be appreciated that following expression of the protein, the signal peptide may be cleaved to yield a mature protein sequence, such as recited in Table 1 herein.

Any appropriate expression vector (e.g., as described in Pouwels et al., Cloning Vectors: A Laboratory Manual (Elsevier, N.Y.: 1985)) and corresponding suitable host can be employed for production of recombinant polypeptides. Expression hosts include, but are not limited to, bacterial species within the genera Escherichia, Bacillus, Pseudomonas, Salmonella, mammalian or insect host cell systems including baculovirus systems (e.g., as described by Luckow et al., Bio/Technology 6:47 (1988)), and established cell lines such as the COS-7, C127, 3T3, CHO, HeLa, and BHK cell lines, and the like. The skilled person is aware that the choice of expression host has ramifications for the type of polypeptide produced. For instance, the glycosylation of polypeptides produced in yeast or mammalian cells (e.g., COS-7 cells) will differ from that of polypeptides produced in bacterial cells, such as Escherichia coli.

### Polypeptides

“Isolated,” when used to describe the various polypeptides disclosed herein, means the polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated protein includes polypeptide in situ within recombinant cells, since at least one component of the polypeptide natural environment will not be present. Ordinarily, however, isolated polypeptide will be prepared by at least one purification step.

A “fragment” is a portion of a polypeptide of the present invention that retains substantially similar functional activity or substantially the same biological function or activity as the polypeptide, which can be determined using assays described herein.

“Percent (%) amino acid sequence identity” or “percent (%) identical” with respect to a polypeptide sequence, i.e. a polypeptide of the invention defined herein, is defined as the percentage of amino acid residues in a candidate sequence that are identical with the amino acid residues in the specific polypeptide of the invention, after aligning the sequences and introducing gaps, if necessary, to achieve the maximum percent sequence identity, and not considering any conservative substitutions as part of the sequence identity.

Those skilled in the art can determine appropriate parameters for measuring alignment, including any algorithms (non-limiting examples described below)needed to achieve maximal alignment over the full-length of the sequences being compared. When amino acid sequences are aligned, the percent amino acid sequence identity of a given amino acid sequence A to, with, or against a given amino acid sequence B (which can alternatively be phrased as a given amino acid sequence A that has or comprises a certain percent amino acid sequence identity to, with, or against a given amino acid sequence B) can be calculated as: percent amino acid sequence identity=X/Y100, where X is the number of amino acid residues scored as identical matches by the sequence alignment program's or algorithm's alignment of A and B and Y is the total number of amino acid residues in B. If the length of amino acid sequence A is not equal to the length of amino acid sequence B, the percent amino acid sequence identity of A to B will not equal the percent amino acid sequence identity of B to A.

In calculating percent identity, typically exact matches are counted. The determination of percent identity between two sequences can be accomplished using a mathematical algorithm. A nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLASTN and BLASTX programs of Altschul et al. (1990) J. Mol. Biol. 215:403. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection. Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite (Invitrogen Corporation, Carlsbad, CA). After alignment of amino acid sequences with ClustalW, the percent amino acid identity can be assessed. A non-limiting example of a software program useful for analysis of ClustalW alignments is GENEDOC™ or JalView (http://www.jalview.org/). GENEDOC™ allows assessment of amino acid (or DNA) similarity and identity between multiple proteins. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG Wisconsin Genetics Software Package, Version 10 (available from Accelrys, Inc., 9685 Scranton Rd., San Diego, CA, USA). When utilizing the ALIGN program for comparing amino acid sequences, a PAM 120 weight residue table, a gap length penalty of 12, and a gap penalty of 4 can be used.

The polypeptide desirably comprises an amino end and a carboxyl end. The polypeptide can comprise D-amino acids, L-amino acids or a mixture of D- and L-amino acids. The D-form of the amino acids, however, is particularly preferred since a polypeptide comprised of D-amino acids is expected to have a greater retention of its biological activity in vivo.

The polypeptide can be prepared by any of a number of conventional techniques. The polypeptide can be isolated or purified from a naturally occurring source or from a recombinant source. Recombinant production is preferred. For instance, in the case of recombinant polypeptides, a DNA fragment encoding a desired peptide can be subcloned into an appropriate vector using well-known molecular genetic techniques (see, e.g., Maniatis et al., Molecular Cloning: A Laboratory Manual, 2nd ed. (Cold Spring Harbor Laboratory, 1982); Sambrook et al., Molecular Cloning A Laboratory Manual, 2nd ed. (Cold Spring Harbor Laboratory, 1989). The fragment can be transcribed and the polypeptide subsequently translated in vitro. Commercially available kits also can be employed (e.g., such as manufactured by Clontech, Palo Alto, Calif.; Amersham Pharmacia Biotech Inc., Piscataway, N.J.; InVitrogen, Carlsbad, Calif., and the like). The polymerase chain reaction optionally can be employed in the manipulation of nucleic acids.

The term “conservative substitution” as used herein, refers to the replacement of an amino acid present in the native sequence in the peptide with a naturally or non-naturally occurring amino acid or a peptidomimetic having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid).

Conservative amino acid substitution tables providing functionally similar amino acids are well known to one of ordinary skill in the art. The following six groups are examples of amino acids that may be considered to be conservative substitutions for one another:
1) Alanine (A), Serine(S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

As naturally occurring amino acids are typically grouped according to their properties, conservative substitutions by naturally occurring amino acids can be determined bearing in mind the fact that replacement of charged amino acids by sterically similar non-charged amino acids are considered as conservative substitutions. For producing conservative substitutions by non-naturally occurring amino acids it is also possible to use amino acid analogs (synthetic amino acids) well known in the art. A peptidomimetic of the naturally occurring amino acid is well documented in the literature known to the skilled person and non-natural or unnatural amino acids are described further below. When affecting conservative substitutions, the substituting amino acid should have the same or a similar functional group in the side chain as the original amino acid.

The phrase “non-conservative substitution” or a “non-conservative residue” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[(—CH2)5-COOH]—CO—for aspartic acid. Non-conservative substitution includes any mutation that is not considered conservative.

A non-conservative amino acid substitution can result from changes in: (a) the structure of the amino acid backbone in the area of the substitution; (b) the charge or hydrophobicity of the amino acid; or (c) the bulk of an amino acid side chain. Substitutions generally expected to produce the greatest changes in protein properties are those in which: (a) a hydrophilic residue is substituted for (or by) a hydrophobic residue; (b) a proline is substituted for (or by) any other residue; (c) a residue having a bulky side chain, e.g., phenylalanine, is substituted for (or by) one not having a side chain, e.g., glycine; or (d) a residue having an electropositive side chain, e.g., lysyl, arginyl, or histadyl, is substituted for (or by) an electronegative residue, e.g., glutamyl or aspartyl.

Alterations of the native amino acid sequence to produce mutant polypeptides, such as by insertion, deletion and/or substitution, can be done by a variety of means known to those skilled in the art. For instance, site-specific mutations can be introduced by ligating into an expression vector a synthesized oligonucleotide comprising the modified site. Alternately, oligonucleotide-directed site-specific mutagenesis procedures can be used, such as disclosed in Walder et al., Gene 42:133 (1986); Bauer et al., Gene 37:73 (1985); Craik, Biotechniques, 12-19 (January 1995); and U.S. Pat. Nos. 4,518,584 and 4,737,462. A preferred means for introducing mutations is the QuikChange Site-Directed Mutagenesis Kit (Stratagene, LaJolla, Calif.).

The terms “N-terminal” and “C-terminal” are used herein to designate the relative position of any amino acid sequence or polypeptide domain or structure to which they are applied. The relative positioning will be apparent from the context. That is, an “N-terminal” feature will be located at least closer to the N-terminus of the polypeptide molecule than another feature discussed in the same context (the other feature possible referred to as “C-terminal” to the first feature). Similarly, the terms “5′-” and “3′-” can be used herein to designate relative positions of features of polynucleotides.

A recombinant polypeptide made in accordance with the methods of the present invention may also be modified by, conjugated or fused to another moiety to facilitate purification of the polypeptides, or for use in immunoassays using methods known in the art. For example, a polypeptide of the invention may be modified by glycosylation, acetylation, phosphorylation, amidation, derivatization by known protecting/blocking groups, proteolytic cleavage, etc.

Modifications contemplated herein include, but are not limited to, modification to side chains, incorporating of unnatural amino acids and/or their derivatives during polypeptide synthesis and the use of crosslinkers and other methods which impose conformational constraints on the polypeptides of the invention. Any modification, including post-translational modification, that introduces flexibility or destabilised the homodimerisation interface of the CTLA4 molecule, but wherein the molecule retains its ability to form a dimer, is contemplated herein. An example includes modification incorporated by click chemistry as known in the art. Exemplary modifications include glycosylation.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivatisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 2a.

TABLE 2

| Non-conventional | | Non-conventional | |
|---|---|---|---|
| amino acid | Code | amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl-Nmbc | | | |
| ethylamino)cyclopropane | | | |

Crosslinkers can be used, for example, to stabilise 3D conformations, using homo-bifunctional crosslinkers such as the bifunctional imido esters having (CH2)n spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

### Binding to CD80 and Liberation of PDL1 Cis-Bound to CD80

It will be appreciated that in preferred embodiments, upon bivalent binding of the fusion proteins of the invention to CD80, PDL1 remains cis-bound to CD80 such that there is substantially no liberation of PDL1 upon binding of the fusion protein to CD80.

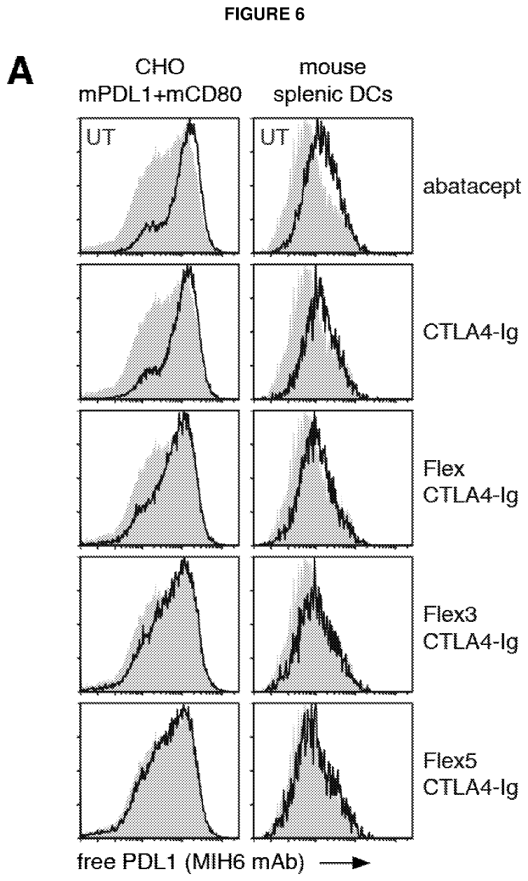

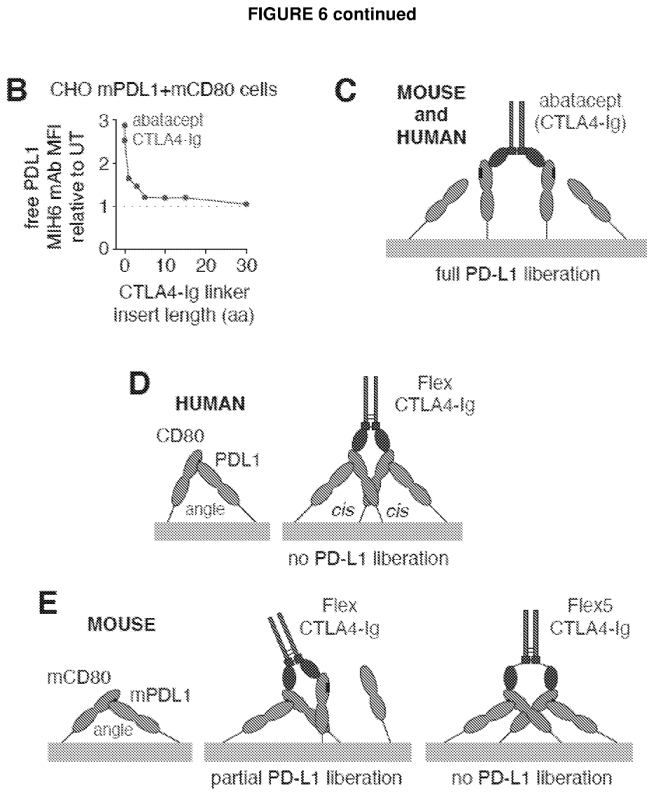

In alternative embodiments, upon bivalent binding, PDL1 is partially liberated from binding to CD80 such that some PDL1 molecules remain cis-bound to CD80. A schematic representation of graded (partial) liberation of PDL1 compared to non-liberation of PDL1 is provided herein in FIG. 6.

As used herein, “substantially remains cis-bound to CD80” refers to a fusion protein for which there is minimal liberation of PDL1 from binding to CD80. Preferably, “substantially remains cis-bound to CD80” suggests that no more than 10%, no more than 9%, no more than 8%, no more than 7%, no more than 6%, no more than 5%, no more than 4%, no more than 3% or no more than 2% of PDL1 is liberated from cis-binding to CD80 upon binding of a fusion protein of the invention.

Where PDL1 is partially liberated from cis-binding to CD80, the percentage of PDL1 molecules liberated is no more than 15%, no more than 20%, no more than 25%, no more than 30%, no more than 40%, no more than 50%, no more than 60%, no more than 70% or no more than 80%, preferably no more than 50%.

The skilled person will be familiar with methods for determining the binding of any fusion protein of the invention to CD80, including according to any of the methods described herein in the Examples.

Moreover, the skilled person will be able to determine the binding of a fusion protein to CD80, including whether a fusion protein of the invention thereby facilitates partial liberation of PDL1 binding to CD80 or substantially no liberation of PDL1 from its binding to CD80. In other words, it will be within the purview of the skilled person to determine whether, upon binding of a fusion protein of the invention to CD80, PDL1 remains bound to CD80. Such methods are also described herein, for example at Examples 1-4.

### Cell Culture

Persons skilled in the art will be familiar with standard methods for transfecting host cells, such as mammalian cells, with a nucleic acid vector and culturing the host cell in suitable conditions for expressing genes encoded by the vector. Representative methods for transfection and culturing of mammalian cells to produce recombinant protein are described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989).

Means for introducing the isolated nucleic acid, vector or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used in accordance with the present invention may be cultured in a variety of media, depending on the cell type used. Commercially available media such as Ham's FI0 (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

Moreover, the skilled person will be familiar with methods for purifying expressed recombinant protein from cell culture media, including using size exclusion and affinity chromatography methods, and combinations thereof.

Where a protein is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The protein prepared from the cells can be purified using, for example, ion exchange, hydroxyapatite chromatography, hydrophobic interaction chromatography, gel electrophoresis, dialysis, affinity chromatography (e.g., lysine affinity column), or any combination of the foregoing. These methods are known in the art and described, for example in WO99/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a poly-histidine tag, e.g., a hexa-histidine tag, or an influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

### Compositions

The recombinant fusion proteins of the invention can be provided in a pharmaceutically acceptable composition for administration to an individual in need thereof. For example the fusion proteins made in accordance with the present invention find utility in the treatment of various conditions in which immunostimulation is desired, such as in the treatment of various cancers, or infectious diseases

Pharmaceutical compositions are also contemplated wherein an CTLA4-Ig fusion protein as disclosed herein, preferably in dimeric form, and one or more additional therapeutically active agents are formulated. Formulations of the CTLA4-Ig fusion proteins disclosed herein are prepared for storage by mixing said CTLA4-Ig fusion protein having the desired degree of purity with optional pharmaceutically acceptable carriers, excipients or stabilizers (Remington's Pharmaceutical Sciences 16th edition, Osol, A. Ed., 1980, incorporated entirely by reference), in the form of lyophilized formulations or aqueous solutions.

Acceptable carriers, excipients, or stabilizers are nontoxic to recipients at the dosages and concentrations employed, and include buffers such as phosphate, citrate, acetate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or CTLA4-Ig fusion proteins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; sweeteners and other flavoring agents; fillers such as microcrystalline cellulose, lactose, corn and other starches; binding agents; additives; coloring agents; salt-forming counter-ions such as sodium; metal complexes (e.g. Zn-protein complexes); and/or non-ionic surfactants such as TWEEN™, PLURONICS™ or polyethylene glycol (PEG).

In one embodiment, the pharmaceutical composition that comprises the CTLA4-Ig fusion proteins disclosed herein may be in a water-soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Some embodiments include at least one of the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine. The formulations to be used for in vivo administration may be sterile. This is readily accomplished by filtration through sterile filtration membranes or other methods.

The CTLA4-Ig fusion proteins disclosed herein may also be formulated as immunoliposomes. A liposome is a small vesicle comprising various types of lipids, phospholipids and/or surfactant that is useful for delivery of a therapeutic agent to a mammal. Liposomes containing the CTLA4-Ig fusion proteins are prepared by methods known in the art. The components of the liposome are commonly arranged in a bilayer formation, similar to the lipid arrangement of biological membranes. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The CTLA4-Ig fusion proteins and other therapeutically active agents may also be entrapped in microcapsules prepared by methods including but not limited to coacervation techniques, interfacial polymerization (for example using hydroxymethylcellulose or gelatin-microcapsules, or poly-(methylmethacylate) microcapsules), colloidal drug delivery systems (for example, liposomes, albumin microspheres, microemulsions, nano-particles and nanocapsules), and macroemulsions. Such techniques are disclosed in Remington's Pharmaceutical Sciences 16th edition, Osol, A. Ed., 1980, incorporated entirely by reference. Sustained-release preparations may be prepared. Suitable examples of sustained-release preparations include semipermeable matrices of solid hydrophobic polymer, which matrices are in the form of shaped articles, e.g. films, or microcapsules. Examples of sustained-release matrices include polyesters, hydrogels (for example poly(2-hydroxyethyl-methacrylate), or poly(vinylalcohol)), polylactides, copolymers of L-glutamic acid and gamma ethyl-L-glutamate, non-degradable ethylene-vinyl acetate, degradable lactic acid-glycolic acid copolymers such as the Lupron Depot® (which are injectable microspheres composed of lactic acid-glycolic acid copolymer and leuprolide acetate), poly-D-(−)-3-hydroxybutyric acid, and ProLease® (commercially available from Alkermes), which is a microsphere-based delivery system composed of the desired bioactive molecule incorporated into a matrix of poly-DL-lactide-co-glycolide (PLG).

### Methods of Treatment

The CTLA4-Ig fusion proteins disclosed herein may find use in a wide range of products. In one embodiment an CTLA4-Ig fusion protein disclosed herein is a therapeutic, a diagnostic, or a research reagent. The CTLA4-Ig fusion proteins may find use in a composition that is monoclonal or polyclonal. The CTLA4-Ig fusion proteins disclosed herein may be used for therapeutic purposes. The CTLA4-Ig fusion proteins may be administered to a patient to treat disorders.

A “patient” for the purposes disclosed herein includes both humans and other animals, e.g., other mammals. Thus the CTLA4-Ig fusion proteins disclosed herein have both human therapy and veterinary applications. The term “treatment” or “treating” as disclosed herein is meant to include therapeutic treatment, as well as prophylactic, measures for a disease or disorder. Thus, for example, successful administration of an CTLA4-Ig fusion proteins prior to onset of the disease results in treatment of the disease. As another example, successful administration of an optimized CTLA4-Ig fusion proteins after clinical manifestation of the disease to combat the symptoms of the disease comprises treatment of the disease. “Treatment” and “treating” also encompasses administration of an optimized CTLA4-Ig fusion proteins after the appearance of the disease in order to eradicate the disease. Successful administration of an agent after onset and after clinical symptoms have developed, with possible abatement of clinical symptoms and perhaps amelioration of the disease, comprises treatment of the disease. Those “in need of treatment” include mammals already having the disease or disorder, as well as those prone to having the disease or disorder, including those in which the disease or disorder is to be prevented.

The dimeric CTLA4-Ig fusion proteins described herein are preferably used to treat a disease or condition in which stimulation of an immune response may be desirable. Such diseases or conditions include cancers, infectious diseases (such as those caused by viruses, fungi, bacteria or other pathogens) and immunodeficiency syndromes.

The dimeric CTLA4-Ig fusion proteins described herein may be used to treat cancer. “Cancer” and “cancerous” herein refer to or describe the physiological condition in mammals that is typically characterized by unregulated cell growth. Examples of cancer include but are not limited to carcinoma, lymphoma, blastoma, sarcoma (including liposarcoma), neuroendocrine tumors, mesothelioma, schwanoma, meningioma, adenocarcinoma, melanoma, and leukemia or lymphoid malignancies.

The CTLA4-Ig fusion proteins described herein, preferably in dimeric form, may be used to treat infectious diseases. By “infectious diseases” herein include diseases caused by pathogens such as viruses, bacteria, fungi, protozoa, and parasites.

Administration of the pharmaceutical composition comprising dimeric CTLA4-Ig fusion protein disclosed herein, e.g., in the form of a sterile aqueous solution, may be done in a variety of ways, including, but not limited to orally, subcutaneously, intravenously, intranasally, intraotically, transdermally, topically (e.g., gels, salves, lotions, creams, etc.), intraperitoneally, intramuscularly, intrapulmonary, vaginally, parenterally, rectally, or intraocularly. In some instances, for example for the treatment of wounds, etc., the CTLA4-Ig fusion protein may be directly applied as a solution or spray. As is known in the art, the pharmaceutical composition may be formulated accordingly depending upon the manner of introduction.

Subcutaneous administration may be used in circumstances where the patient may self-administer the pharmaceutical composition. Many protein therapeutics are not sufficiently potent to allow for formulation of a therapeutically effective dose in the maximum acceptable volume for subcutaneous administration. This problem may be addressed in part by the use of protein formulations comprising arginine-HCl, histidine, and polysorbate. CTLA4-Ig fusion proteins disclosed herein may be more amenable to subcutaneous administration due to, for example, increased potency, improved serum half-life, or enhanced solubility. As is known in the art, protein therapeutics are often delivered by IV infusion or bolus. The CTLA4-Ig fusion proteins disclosed herein may also be delivered using such methods. For example, administration may be by intravenous infusion with 0.9% sodium chloride as an infusion vehicle.

Pulmonary delivery may be accomplished using an inhaler or nebulizer and a formulation comprising an aerosolizing agent. For example, AERx® inhalable technology commercially available from Aradigm, or Inhance™ pulmonary delivery system commercially available from Nektar Therapeutics may be used. Furthermore, CTLA4-Ig fusion proteins disclosed herein may be amenable to oral delivery.

In addition, any of a number of delivery systems are known in the art and may be used to administer the CTLA4-Ig fusion proteins disclosed herein. Examples include, but are not limited to, encapsulation in liposomes, microparticles, microspheres (e.g., PLA/PGA microspheres), and the like. Alternatively, an implant of a porous, non-porous, or gelatinous material, including membranes or fibers, may be used. Sustained release systems may comprise a polymeric material or matrix such as polyesters, hydrogels, poly(vinylalcohol), polylactides, copolymers of L-glutamic acid and ethyl-L-gutamate, ethylene-vinyl acetate, lactic acid-glycolic acid copolymers such as the Lupron Depot®, and poly-D-(−)-3-hydroxyburyric acid. It is also possible to administer a nucleic acid encoding an CTLA4-Ig fusion protein disclosed herein, for example by retroviral infection, direct injection, or coating with lipids, cell surface receptors, or other transfection agents. In all cases, controlled release systems may be used to release the CTLA4-Ig fusion protein disclosed herein, at or close to the desired location of action.

The dosing amounts and frequencies of administration are, in one embodiment, selected to be therapeutically or prophylactically effective. As is known in the art, adjustments for protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

The concentration of the therapeutically active dimeric CTLA4-Ig fusion proteins in the formulation may vary from about 0.1 to 100 weight %. In one embodiment, the concentration of the CTLA4-Ig fusion proteins is in the range of 0.003 to 1.0 molar. In order to treat a patient, a therapeutically effective dose of the CTLA4-Ig fusion protein disclosed herein may be administered. By “therapeutically effective dose” herein is meant a dose that produces the effects for which it is administered. The exact dose will depend on the purpose of the treatment, and will be ascertainable by one skilled in the art using known techniques. Dosages may range from 0.0001 to 100 mg/kg of body weight or greater, for example 0.1, 1, 10, or 50 mg/kg of body weight. In one embodiment, dosages range from 1 to 10 mg/kg.

In some embodiments, only a single dose of the dimeric CTLA4-Ig fusion protein is used. In other embodiments, multiple doses of the dimeric CTLA4-Ig fusion protein are administered. The elapsed time between administrations may be less than 1 hour, about 1 hour, about 1-2 hours, about 2-3 hours, about 3-4 hours, about 6 hours, about 12 hours, about 24 hours, about 48 hours, about 2-4 days, about 4-6 days, about 1 week, about 2 weeks, or more than 2 weeks.

In other embodiments the CTLA4-Ig fusion proteins disclosed herein are administered in metronomic dosing regimens, either by continuous infusion or frequent administration without extended rest periods. Such metronomic administration may involve dosing at constant intervals without rest periods. Typically such regimens encompass chronic low-dose or continuous infusion for an extended period of time, for example 1-2 days, 1-2 weeks, 1-2 months, or up to 6 months or more. The use of lower doses may minimize side effects and the need for rest periods.

### EXAMPLES

### Example 1: Materials and Methods

### Surface Protein Cloning

Mouse or human cDNA was prepared using a Verso cDNA Synthesis Kit (Thermo Fisher Scientific). Open reading frames were PCR amplified using Q5 High Fidelity DNA Polymerase (New England BioLabs). Primers incorporated EcoRI or Xhol restriction sites allowing directional cloning into MSCV-IRES-eBFP2. The primer sequences (as shown in Table 1) were used, based on the indicated transcript variants.

mCD80 and hCTLA4Y201A were cloned into MSCV-IRES-eBFP2 using NEBuilder HiFi DNA Assembly (New England BioLabs), with the Y201A mutation introduced in the PCR primers. All vectors were sequence verified against the NCBI Reference Sequence Database. Sequences encoding membrane-bound anti-human CD3 single chain antibody fragment (OKT3-scFv) were based on the previously described CD5L-OKT3-scFv-CD14 sequence (NCBI GenBank HM208750.1). Its human codon optimised open reading frame was ordered as a gBlock (Integrated DNA Technologies) and cloned into MSCV-IRES-mCherry using NEBuilder HiFi DNA Assembly (New England BioLabs).

### Fluorescent Fusion Protein Cloning

CD80mCherry was generated by fusing full length hCD80 (NP_005182.1) residues 1-288 to a Gly/Ser linker (2×SGGGG) followed by monomeric mCherry. A Xhol restriction site encoding Leu-Glu was included between the C terminus of hCD80 and the first Ser of the linker. PDL1mGFP was generated by fusing full length hPDL1 (NP_054862.1) residues 1-290 to the same Gly/Ser linker, with the same intervening Xhol site, followed by monomeric mEGFP. Sequences encoding both fusion proteins were cloned into MSCV-Puro.

### Recombinant CTLA4-Ig Variant Cloning

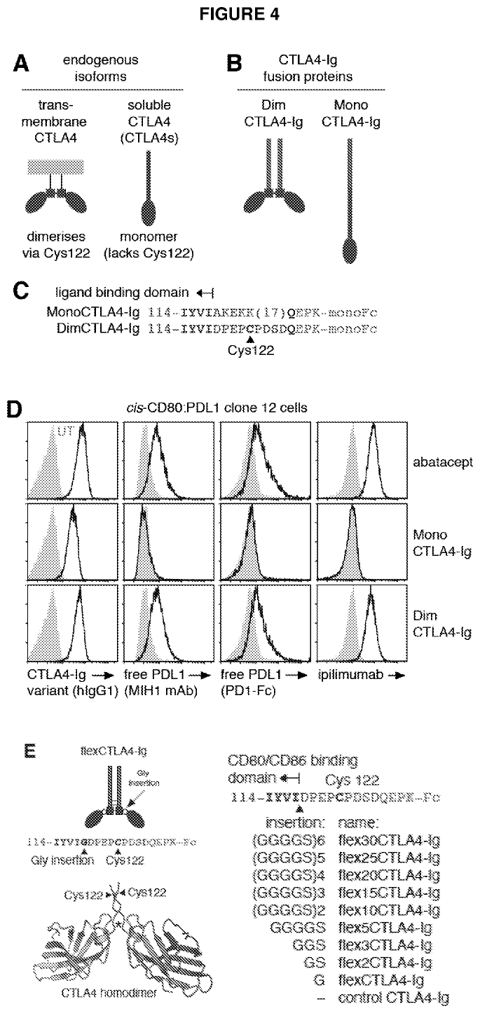

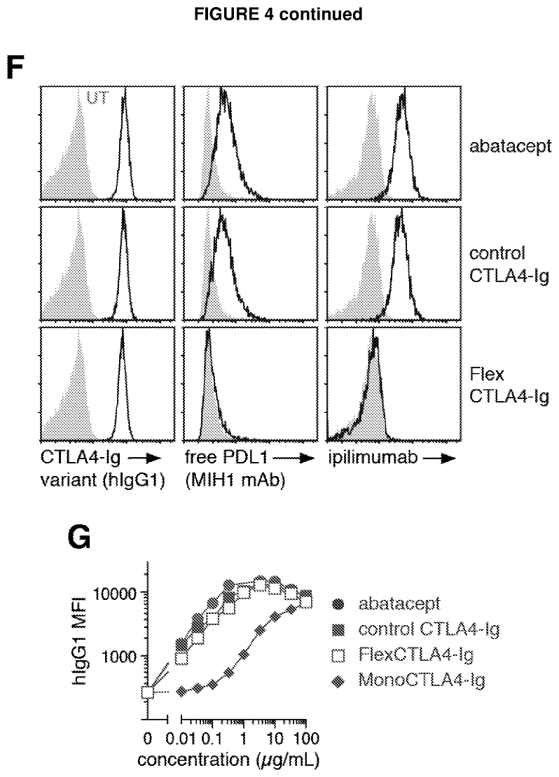

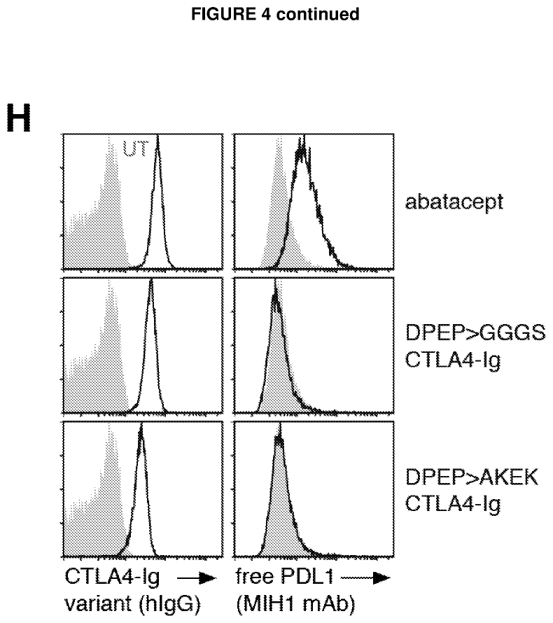

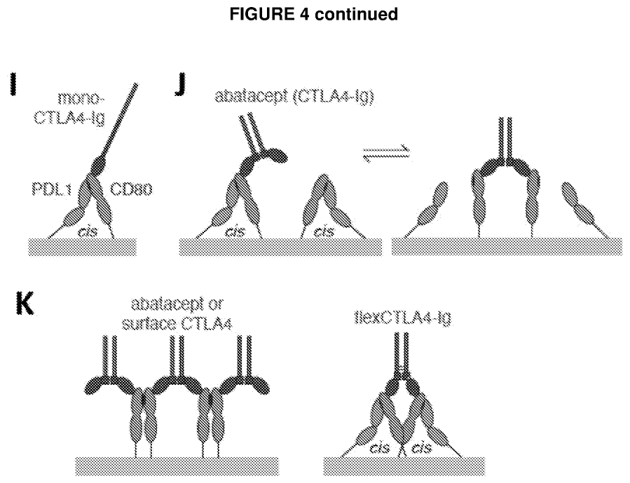

CTLA4-Ig variant sequences were cloned into Xhol/BgIII digested pCAGGS using NEBuilder HiFi DNA Assembly (New England BioLabs). All vectors incorporated a consensus Kozak translation initiation sequence upstream of the human oncostatin M signal peptide (residues 1-25 of NP_065391.1) followed by human CTLA4 sequences, human IgG1 Fc sequences, and a C-terminal FLAG tag. MonoCTLA4-Ig includes residues 37-174 of the soluble sCTLA4 isoform (NP_001032720.1) and dimCTLA4-Ig includes residues 37-161 of the transmembrane CTLA4 isoform (NP_005205.2). DimCTLA4-Ig sequences were ordered as a gBlock (Integrated DNA Technologies), modifying codons within the IgG1 hinge region to reduce G/C content. All CTLA4-Ig variants include a single Gln residue between the last residue of the CTLA4 ectodomain and the first residue of the IgG1 hinge as described for abatacept. MonoCTLA4-Ig and dimCTLA4-Ig include human IgG1 residues Glu216-Gly446 (IgG1 numbering as previously described) with IgG1 hinge domain C220S, C226S, and C229S mutations as described for abatacept and a monoFc region including S364N, Y407N, and K409T mutations that promote N-glycosylation and prevent Fc dimerisation. An IgG1 P238S substitution was also introduced to match abatacept protein sequence. FlexCTLA4-Ig variants were generated by inserting flexible linker peptides between IIe117 and Asp118 of CTLA4, where the soluble and transmembrane CTLA4 isoforms diverge (FIG. 4C). To generate flexCTLA4-Ig variants with varying Gly/Ser linker lengths, a gBlock was designed to encode the longest linker (6×GGGGS repeats) but incorporating different modified codons within each GGGGS repeat to allow subsequent PCR amplification of fragments encoding shorter linkers (5×GGGGS, 4×GGGGS, etc). PCR products were cloned into pCAGGS using NEBuilder HiFi DNA Assembly (New England BioLabs) as described above. FlexCTLA4-Ig variants and their matched control CTLA4-Ig contain human IgG1 with a C220S mutation (IgG1 numbering as previously described) but with other IgG1 hinge residues Cys226 and Cys229 intact to promote Fc dimerisation.

### Cell Culture

Cells were cultured at 37° C. in a 10% CO₂ incubator in medium containing 100 U/mL penicillin, 100 μg/mL streptomycin (Gibco), and 10% FCS (Sigma-Aldrich). Base medium for CHO cells was α-Minimal Essential Medium (α-MEM; Gibco), and for 293T cells Dulbecco's Modified Eagle Medium (DMEM; Gibco). Cells were detached for passaging or harvest with Trypsin-EDTA (Gibco). For retrovirus production, expression and packaging vectors were introduced into 293T cells by calcium phosphate transfection, and retroviral transduction of CHO cells was performed using standard protocols.

### Recombinant Fc Fusion Proteins

The following laboratory grade mouse Fc fusion proteins were used: mCTLA4-Fc (mouse IgG1, Absolute Antibody), mPD1-Fc (mouse IgG2a, Absolute Antibody), mCD28-Fc (human IgG1-6His, Sino Biological). The following commercial human Fc fusion proteins were used: CTLA4-Fc (human IgG1, Absolute Antibody), PD1-Fc (mouse IgG2a, Sapphire Bioscience). Clinical grade abatacept (Bristol-Myers Squibb) was used. Mouse IgG1 and mouse IgG2a Fc fusion proteins were respectively detected with anti-mIgG1-APC or -FITC (clone RMG1-1, BioLegend) or anti-mIgG2a-APC or -PE-Cγ7 (clone m2a-15F8, Thermo Fisher). Human IgG1 fusion proteins were detected with anti-hIgG1-APC (polyclonal, R&D Systems).

### Cell-Cell Interaction Assays

Cells were harvested using TrypLE Express Dissociation Reagent (Gibco), before staining with 1.5 μM of either Cell Trace Far Red (CTFR) or carboxyfluorescein succinimidyl ester (CFSE) following manufacturer protocols. Following staining, 1.5×10⁵ cells from each stained population were incubated together in a 96 well U-bottom plate in a final volume of 200 μL of interaction assay buffer comprising 10% (v/v) heat-inactivated FCS, 50 μM EDTA, and 1% (v/v) HEPES in PBS. For assays using CD28 or CTLA4 expressing cells, 1×10⁵ unlabelled, CFSE and CTFR cells each were incubated together in a 96 well plate. Where required, blocking antibodies were immediately added to combined cells: either 50 μg/mL anti-hCD28 (clone TGN1412), 0.625 μg/mL ipilimumab (Bristol-Myers Squibb) or 0.5 μg/mL nivolumab (Bristol-Myers Squibb). The Fc fusion proteins abatacept, hCTLA4-Fc, or CD28-Fc were added concurrently at 50 μg/mL. For cell-cell interaction assays, cells were spun at 20g for 1 min, before incubating at 37° C., 10% CO₂ for 45 min. Cells were analysed on an LSR Fortessa (BD Biosciences) using the High Throughput Sampler with acquisition settings of 0.5 μL/s sample flow rate, 100 μL sample and mixing volume, 50 μL/s mixing speed, and 2 mixes per sample. For co-culture experiments, stained cells were incubated together in the presence of the relevant blocking Abs, as well as either anti-hPDL1-PE (0.5 μg/mL, clone MIH1) or hPD1-Fc (100 μg/ml) and anti-mIgG2a-APC or -PE-Cγ7 (50 g/mL). Cells were incubated together at 37° C. and 10% CO₂ for 45 min, and analysed on either LSR II or LSR Fortessa instruments (BD Biosciences).

### Flow Cytometry

Harvested cells were washed with PBS. To assess binding of fusion proteins, cells were incubated with 20 μL of 10 μg/mL fusion protein for 20 min unless otherwise stated, then stained with the relevant anti-Fc secondary antibody for a further 20 min. After washing to remove excess fusion protein and antibody, cell pellets were resuspended in 100 μL of FACS buffer (PBS with 10% FCS). Flow cytometry was performed on viable cells (based on SSC/FSC) using a BD LSRII or LSRFortessa (BD Biosciences), and cell sorting was performed using a BD Influx (BD Biosciences) with a 100 μm nozzle. Mouse antigens were detected using the following antibodies: mPDL1-PE-Cγ7 (clone MIH5, Thermo Fisher), mPDL1-AF647 (clone MIH6, Bio-Rad), mCD80-APC (clone 16-10A1, Thermo Fisher), mCD80-PE (clone 1G10, Thermo Fisher), mPD1-PerCP-Cγ5.5 (clone RMP1-30, BioLegend), and mPD1-BV711 (clone 29F1A12, BioLegend). Human antigens were detected using the following antibodies: PDL1-PE (clone MIH1, Thermo Fisher), CD80-BV711 (clone L307.4, Thermo Fisher), CD28-PerCP-Cγ5.5 (clone CD28.2, Biolegend), CD4-BV711 (clone SK3, BioLegend), and CD8-PE-Cγ7 (clone SK1, BioLegend). All antibodies were used at a final dilution of 1:200 from stock. Human CTLA4 was detected with ipilimumab directly conjugated to CF647 using Mix-n-Stain CF647 antibody labelling kit (Merck).

### Transendocytosis Assay

A 2:1 ratio of cis-CD80mCherry:PDL1mGFP clone cells to CD28, CTLA4WT, or CTLA4Y201A cells were co-cultured together for various periods. Cultures were harvested and the suspension thoroughly mixed to disaggregate interacting cells. Samples were stained following the PD1-Fc flow cytometry staining protocol. Flow cytometry gating was used to restrict analysis to singlet cis-CD80mCherry:PDL1mGFP cells, excluding BFP+CD28, CTLA4WT, or CTLA4Y201A cells. For ipilimumab treatment of 24 h transendocytosis co-cultures, CD28 and CTLA4 cells were plated in media containing 1 μg/mL ipilimumab for 5 mins prior to addition of cis-CD80mCherry:PDL1mGFP cells.

### Protein Expression and Purification

Freestyle™ 293-F cells (Thermo Fisher Scientific) were grown to a density of 2×10⁶/mL in FreeStyle 293 expression medium and transiently transfected with plasmid DNA and polyethyleneimine (PEI) at a 3:1 PEI-DNA ratio (1 mg DNA per litre). Cells were grown for 7 days after transfection, supplemented with Glutamax (Thermo Fisher Scientific), 0.2 mM butyric acid (Sigma-Aldrich) and 5 g/L lupin (Solabia) 1 and 4 days after transfection. Secreted recombinant protein was purified using Protein G resin (GE Life Sciences). The protein was subsequently concentrated and applied to a Superdex 200 size exclusion column (GE Life Sciences) equilibrated in TBS, pH 7.5.

### Example 2: Abatacept (CTLA4-Ig) Disrupts Cell Surface Cis-CD80:PDL1 Interactions and Liberates PDL1

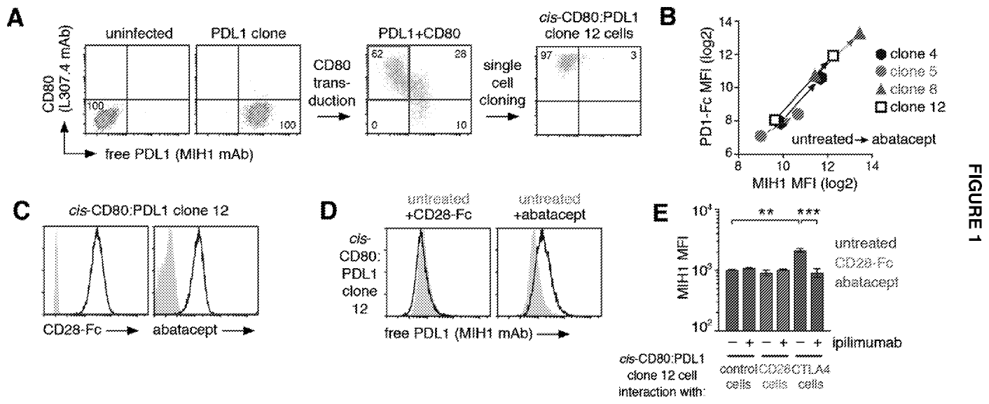

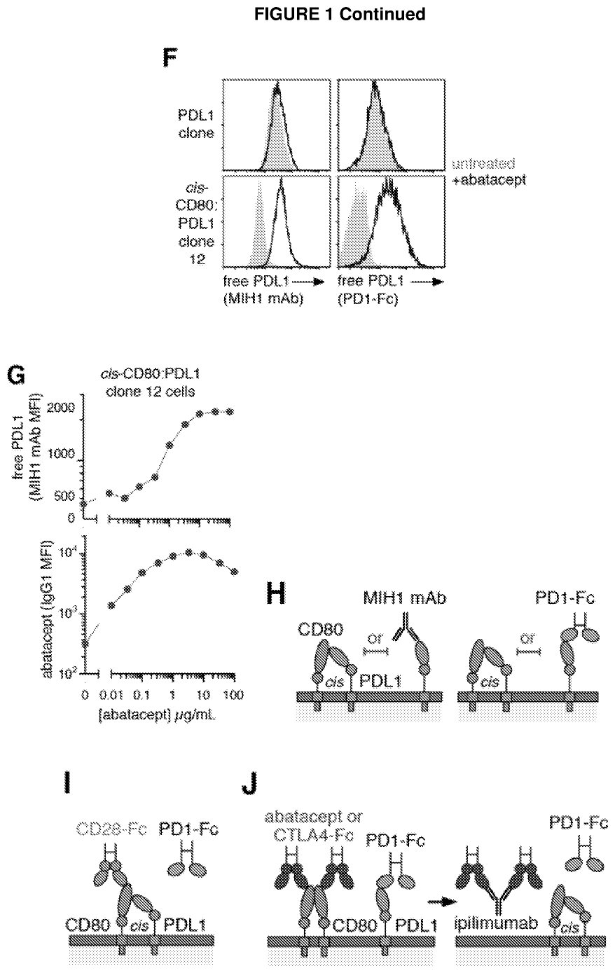

To investigate cell surface cis-CD80:PDL1 interactions the inventors retrovirally expressed human PDL1 in Chinese hamster ovary (CHO) cells, isolated a PDL1^{high} clone, then retrovirally expressed CD80 (FIG. 1A). This produced a population of CD80^{high}PDL1^{high} cells with reduced binding of the anti-PDL1 monoclonal antibody MIH1, indicating that cis-CD80:PDL1 interactions prevent MIH1 binding^{4-6,38} (FIGS. 1A, and H). Single cell sorting produced several CD80^{high}PDL1^{high}/MIH1^{low} clones where binding of recombinant PD1-Fc closely correlated with MIH1 staining, verifying that MIH1 only recognises free PDL1 (FIG. 1A-B, H). Importantly, free surface PDL1 measured by MIH1 or PD1-Fc remained low during continuous culture of these “cis-CD80:PDL1” clones.

To mimic surface CD80 interactions with its receptors CD28 or CTLA4, cis-CD80:PDL1 (clone 12) cells were cultured with recombinant CD28-Fc or abatacept (CTLA4-Ig). These fusion proteins comprise disulphide-linked homodimers resembling the ligand-binding ectodomains of native membrane-bound receptors. CD28-Fc binding to cis-CD80:PDL1 cells did not affect MIH1 or PD1-Fc staining (FIG. 1C-E). In contrast, abatacept binding to cis-CD80:PDL1 cells triggered a significant and population-wide increase in free PDL1 evidenced by closely correlated MIH1 or PD1-Fc staining (FIG. 1B-F). PD1-Fc blockade with the clinical anti-PD1 antibody nivolumab verified assay specificity (not shown). Clinical grade abatacept and laboratory grade CTLA4-Ig reproducibly liberated PDL1 in bulk CD80⁺PDL1⁺ cells and in independent cis-CD80:PDL1 clones (data not shown). Human CTLA4 binds mouse CD80, and accordingly abatacept also induced PDL1 liberation in cells co-expressing murine PDL1 and CD80 orthologs (data not shown). These results are the first to demonstrate that abatacept binding to CD80 displaces cis-bound PDL1, which can then bind PD1.

Importantly, abatacept induces PDL1 liberation at doses as low as 1 μg/ml with maximum effects plateauing at 10 μg/mL (FIG. 1G), below the effective serum concentration in arthritis patients. Notably, ipilimumab prevented abatacept-induced PDL1 liberation (FIG. 1E). Hence, direct blockade of CTLA4:CD80 interactions by ipilimumab can indirectly disengage the PD1 checkpoint by promoting cis-CD80:PDL1 interactions (FIG. 11-J).

### Example 3: CTLA4-Induced PDL1 Liberation Promotes Cell-Cell Interaction Through PDL1:PD1

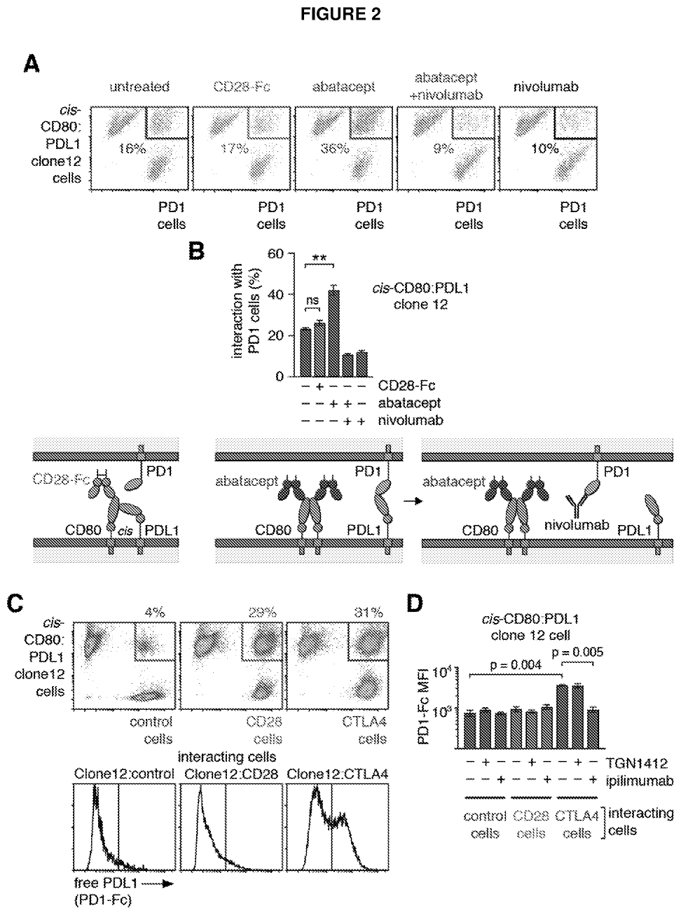

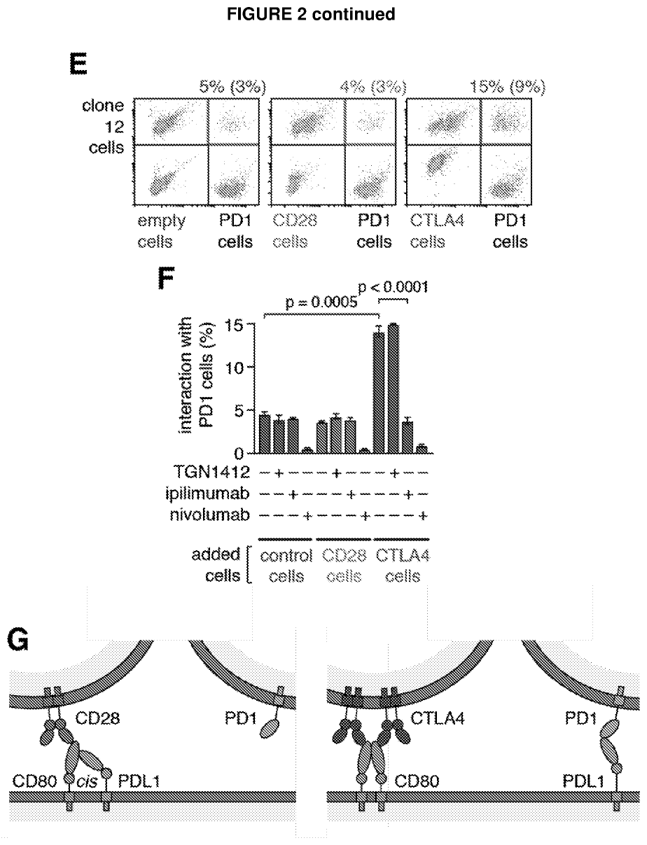

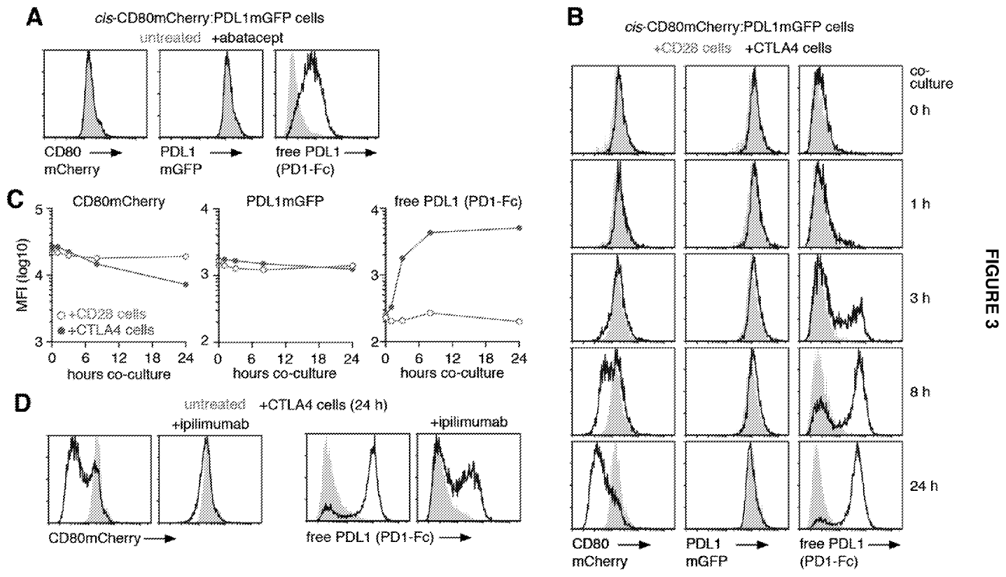

When T cells encounter cognate APCs, trans interactions between surface proteins at the immunological synapse determine whether T cells are activated or anergised. Using flow cytometry the inventors examined associations between CHO cells stably expressing APC or T cell surface proteins. Positive control interactions between PDL1 cells and PD1 cells were disrupted by nivolumab (data not shown). Compared to parental PDL1-only cells, cis-CD80:PDL1 cell interactions with PD1 cells were reduced, consistent with lower free surface PDL1 (data not shown). Whereas CD28-Fc treatment had no effect, abatacept significantly increased the proportion of cis-CD80:PDL1 cells interacting with PD1 cells in a nivolumab-sensitive manner (FIG. 2A-B). Abatacept triggered similar interactions between cells expressing the corresponding mouse proteins (data not shown). Hence, abatacept-induced PDL1 liberation from cis-CD80:PDL1 complexes promotes PDL1 interactions with surface PD1 on neighbouring cells (FIG. 2B).

### Example 4: Cell Surface CTLA4 Induces PDL1 Liberation

The inventors then tested whether cell surface, membrane-bound CTLA4 similarly disrupts cis-CD80:PDL1 complexes on interacting cells. The inventors generated CHO cells stably expressing surface CD28 or CTLA4 with a cytoplasmic domain Y201A mutation that promotes surface retention (data not shown). Co-culture of CFSE-labelled CD28 or CTLA4 cells with CTFR-labelled cis-CD80:PDL1 clone 12 cells resulted in robust cell-cell interactions that were respectively disrupted by TGN1412 antibody blockade of CD28 or ipilimumab blockade of CTLA4 (FIG. 2C). Whereas free surface PDL1 on cis-CD80:PDL1 clone 12 cells was unaltered by CD28 cell binding, CTLA4 cells induced significant PDL1 liberation allowing PD1-Fc binding (FIG. 2C-D) or MIH1 staining (not shown). As an orthogonal approach the inventors added unlabelled CTLA4 cells to co-cultures of CTFR-labelled cis-CD80:PDL1 clone 12 cells and CFSE-labelled PD1 cells. ‘Third party’ CTLA4 cells significantly increased cis-CD80:PDL1 cell interactions with PD1 cells, in an ipilimumab and nivolumab-sensitive manner (FIG. 2E-F). These results demonstrate that dimeric CTLA4 in its native membrane-bound form liberates PDL1 from cis-CD80:PDL1 complexes on interacting cells, enabling PDL1 ligation of surface PD1 on neighbouring cells (as depicted in the schematic in FIG. 2G).

### Example 5: CTLA4-Mediated Transendocytosis of CD80 Releases Free PDL1

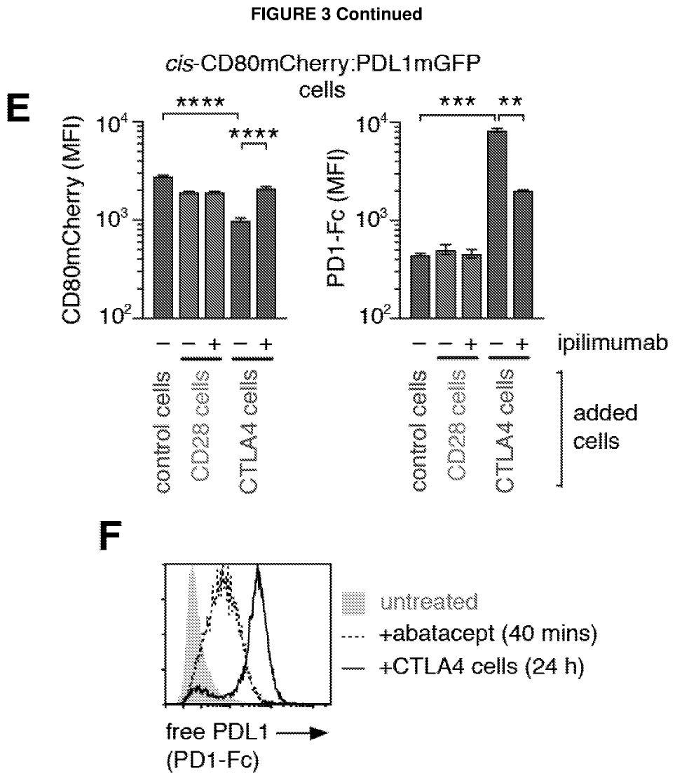

Membrane-bound CTLA4 can capture its ligands CD80 and CD86 from opposing cells through transendocytosis, in immune cells or CHO cell models. To visualise this the inventors stably co-transduced CHO cells with vectors encoding the CD80 ectodomain fused to intracellular mCherry and the PDL1 ectodomain fused to intracellular monomeric green fluorescent protein (mGFP). Flow sorting yielded a cis-CD80mCherry:PDL1mGFP clone where abatacept treatment induced marked PDL1 liberation without affecting CD80mCherry or PDL1mGFP protein levels (FIG. 3A). Using singlet gated flow cytometry the inventors examined cis-CD80mCherry:PDL1mGFP cells co-cultured with cells expressing surface CD28 or wild type CTLA4. Whereas CD28 cells had no effect, CTLA4 cells markedly depleted CD80mCherry from co-cultured cis-CD80mCherry:PDL1mGFP cells (FIG. 3B-C). This was accompanied by dramatic increases in free surface PDL1 measured by PD1-Fc binding, despite unaltered PDL1mGFP levels (FIG. 3B-C). Cells expressing wild type CTLA4 or CTLA4Y201A induced potent PDL1 liberation (data not shown), which was prevented by ipilimumab (FIG. 3D-E). Hence, CTLA4-induced transendocytic depletion of CD80 from cis-CD80:PDL1 complexes results in cells bereft of costimulatory ligand but with abundant free coinhibitory ligand, double-locking an inhibitory state. In contrast to previous findings where PDL1 overexpression on APCs protected CD80 from CTLA4-mediated depletion, the inventors' results suggest that CTLA4 binding and transendocytosis of CD80 is a dominant mechanism that drives PDL1 liberation. Furthermore, CTLA4 cells appear to liberate PDL1 more potently than CTLA4-Ig (FIG. 3F).

### Example 6: Monomeric CTLA4 binds CD80 without displacing PDL1

Endogenous CTLA4 has two isoforms: a transmembrane disulphide-linked homodimer, and a secreted/soluble monomer (sCTLA4) lacking the transmembrane domain (FIG. 4A). To examine sCTLA4 interactions with surface cis-CD80:PDL1 the inventors produced recombinant monoCTLA4-Ig comprising sCTLA4 fused to a human IgG1 Fc domain harboring point mutations that prevent dimerisation. Control dimeric CTLA4-Ig (dimCTLA4-Ig) was made by fusing monomeric Fc to the transmembrane CTLA4 ectodomain that includes Cys 122 involved in covalent homodimerisation (FIG. 4B-C). MonoCTLA4-Ig and dimCTLA4-Ig behaved as monomers and dimers respectively by non-reducing SDS-PAGE (data not shown), and bound cis-CD80:PDL1 cells (FIG. 4D). Whereas abatacept and dimCTLA4-Ig (effectively in-house abatacept) induced population-wide PDL1 liberation as anticipated, monoCTLA4-Ig binding to cis-CD80:PDL1 cells had no effect (FIG. 4D). In light of recent reports that PDL1 binds in cis to the homodimer interface of CD80, opposite its CTLA4/CD28 binding face, the inventors' results suggest that monomeric CTLA4 forms a tripartite complex with surface cis-CD80:PDL1.

### Example 7: Dimeric Flexible CTLA4-Ig Variants do not Liberate CD80-Bound PDL1

Since these results indicate that monoCTLA4-Ig does not liberate PDL1, the inventors reasoned that dimeric CTLA4-Ig with longer ‘arms’ may retain bivalent CD80 binding without liberating PDL1. To test this the inventors inserted flexible Gly/Ser linkers of different lengths adjacent to the ligand-binding domain of CTLA4-Ig, also displacing several highly conserved CTLA4 residues (Asp118 to Pro121) previously implicated in rigid-body homodimerisation (FIG. 4E). Overall fusion homodimerisation was maintained by CTLA4 Cys122 (numbered Cys120 in SEQ ID NO: 2) and two IgG1 hinge domain cysteines (data not shown). This series of variants, designated flex (n) CTLA4-Ig (n=30, 25, 20, 15, 10, 5, 3, 2, or 1 inserted Gly/Ser residues; bound cis-CD80:PDL1 cells but failed to liberate PDL1, in contrast to an otherwise-identical wild type control CTLA4-Ig or abatacept (FIG. 4F). Indeed a single Gly insertion (flexCTLA4-Ig) at the CTLA4 homodimerisation interface disabled its PDL1 liberation activity (FIG. 4E-F). The PDL1 liberation activity of CTLA4-Ig was also disabled by substituting the CTLA4 ‘DPEP’ peptide (Asp188 to Pro121 inclusive) with either a flexible ‘GGGS’ linker peptide or an ‘AKEK’ peptide derived from the monomeric soluble isoform of CTLA4 (FIG. 4H).

Binding of flexCTLA4-Ig to cis-CD80:PDL1 cells was comparable to control CTLA4-Ig and abatacept, suggesting a high-avidity bivalent interaction (FIG. 4G). To verify this the inventors incubated fusion proteins with cis-CD80:PDL1 cells, then cells were washed and unoccupied ligand-binding domains of bound fusion proteins were detected with fluorescently-conjugated ipilimumab (anti-CTLA4). Monovalent binding of monoCTLA4-Ig precluded ipilimumab association (FIGS. 4D and 4I). Surprisingly, cis-CD80:PDL1 cells pre-incubated with wild type CTLA4-Ig proteins such as abatacept or dimCTLA4-Ig showed ipilimumab staining (FIGS. 4D and 4F), indicating some fusion binding was monovalent (FIG. 4J, left). In contrast, abatacept binding to CD80-only cells was uniformly bivalent (data not shown). These results suggest that PDL1 cis interactions with CD80 may partially inhibit the bivalent bridging of CD80 monomers by abatacept. Indeed at low concentrations (0.05-0.5 μg/mL) abatacept binds cis-CD80:PDL1 cells without liberating PDL1 (FIG. 1G), presumably due to monovalent interaction (FIG. 4J, left). This is consistent with a previous report that PDL1 overexpression can inhibit CD80: CTLA4 interactions. However at therapeutically relevant concentrations (>1 μg/mL) the inventors instead find that abatacept dominantly induces PDL1 liberation (FIG. 1G), potentially by shifting the equilibrium towards bivalent crosslinking that reorients CD80 at the expense of PDL1 binding (FIG. 4J, right). Notably, binding of flexCTLA4-Ig variants to cis-CD80:PDL1 cells precluded subsequent detection with labelled ipilimumab (FIG. 4F). This was not due to altered ipilimumab recognition, because pre-incubation with ipilimumab prevented flexCTLA4-Ig cell binding (data not shown). Hence, in contrast to abatacept, flexCTLA4-Ig binding to surface cis-CD80:PDL1 complexes is uniformly bivalent. This implies PDL1 liberation not only requires bivalent CTLA4 binding to CD80, but specific CD80 reorientation enforced by the CTLA4 homodimer arrangement. In crystal structures CD80 and CTLA4 form a linear daisy chain of alternating bivalent homodimers with each rod-shaped CD80 molecule in an ‘upright’ arrangement (FIG. 4K, left). The inventors' flexCTLA4-Ig results suggest that relaxing the rigid-body structural constraints between the two CTLA4 ligand-binding domains allows bivalent CD80 binding without obligatory CD80 reorganisation, allowing cis-PDL1 to remain bound (FIG. 4K, right).

### Example 8: Characterisation of the Nature of Binding of “flexCTLA4-Ig” Compared to Abatacept

The inventors characterised the binding of various fusion proteins to cis-CD80:PDL1 complexes. More specifically, CTLA4-Ig variant was incubated with cells that express cis-CD80:PDL1 complexes, the cells were washed and the association of the fusion protein was tracked over time by anti-IgG1 flow cytometry.

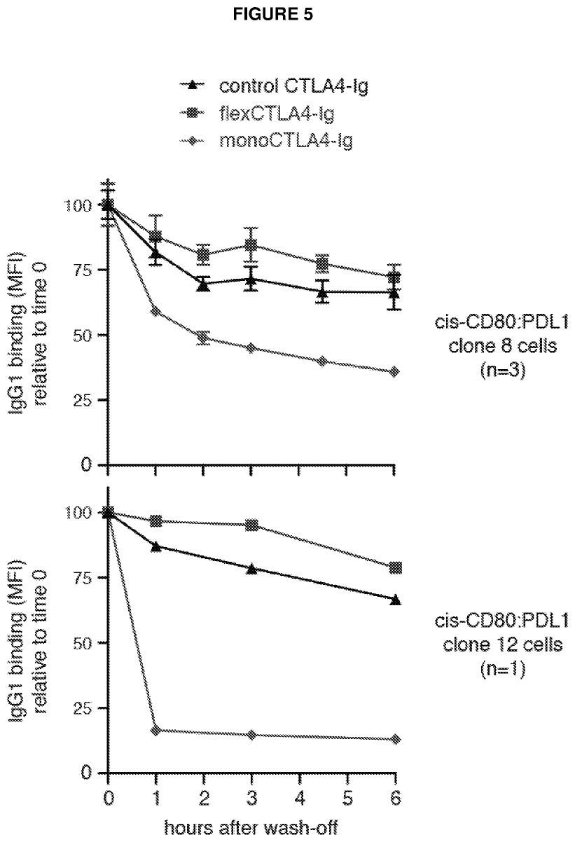

In two independent cis-CD80:PDL1 cell clones, the dissociation of flexCTLA4-Ig was observed to be slower than for control CTLA4-Ig (“in-house abatacept”). In both clones monoCTLA4-Ig dissociation is more rapid, consistent with its inherent monovalent binding (FIG. 5). The results of this assay are consistent with the inventors' observations from previous ipilimumab co-incubation data suggesting that control CTLA4-Ig (essentially abatacept) binding to cis-CD80:PDL1 complexes is in equilibrium between monovalent (low affinity) and bivalent (high affinity/avidity) binding, whereas flexCTLA4-Ig always binds bivalently and is therefore is less likely to dissociate once bound. The results are shown in FIG. 5.

### Results

FlexCTLA4-Ig Variants Induced Graded Liberation of Murine mPD-L1 from Cis-mCD80

In contrast to the inventors' observations with human cis-CD80:PD-L1 complexes, incubation of the Flex (n) CTLA4-Ig series with CHO cells expressing mouse mCD80 and mPD-L1 revealed a graded response where PD-L1 liberating activity was partially retained by FlexCTLA4-Ig (single Gly insertion) and only fully disabled with linker lengths of 5 residues or more (FIG. 6A-B). Similar results were obtained with mouse primary splenic DCs (FIG. 6A). These results suggested potential differences in cis-CD80:PDL1 complex topology between mouse and human (FIG. 6C-E) and raise the possibility of tuning immune responses through the use of different CTLA4-Ig linkers.

### CTLA4-Induced PD-L1 Liberation is Essential for Immunosuppression by Abatacept

In mouse arthritis models and patients with RA or idiopathic arthritis, disease amelioration by abatacept is thought to be mediated by blockade of the costimulatory ligands CD80 and CD86 on APCs. To assess whether PD-L1 liberation by CTLA4-Ig also contributes to immunosuppression, the inventors firstly adapted FlexCTLA4-Ig and matched control CTLA4-Ig for in vivo use. Abatacept harbors mutations in IgG1 hinge domain cysteines that disable Fc receptor binding and minimise Fc-dependent cytotoxicity. Since these cysteines were reinstated in the Flex (n) CTLA4-Ig variant series to ensure covalent homodimerisation, to impair their Fc receptor binding, the inventors introduced Fc “LALA” mutations.

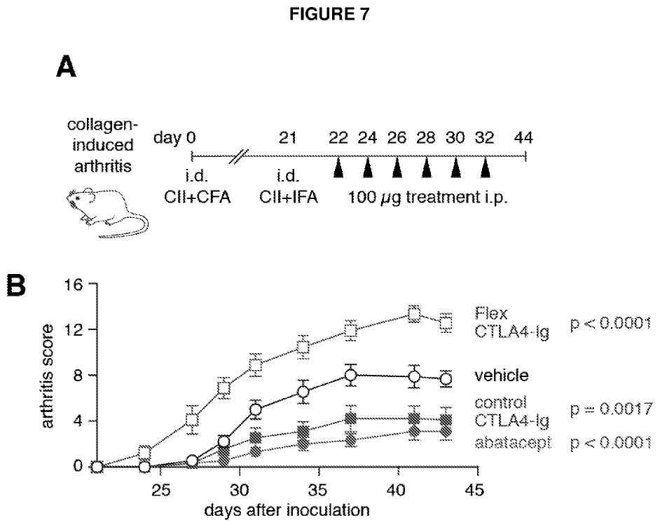

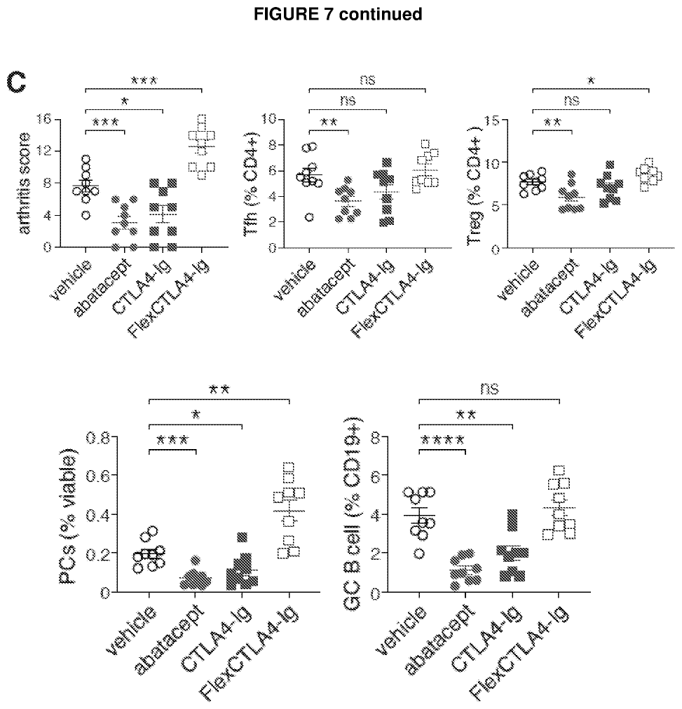

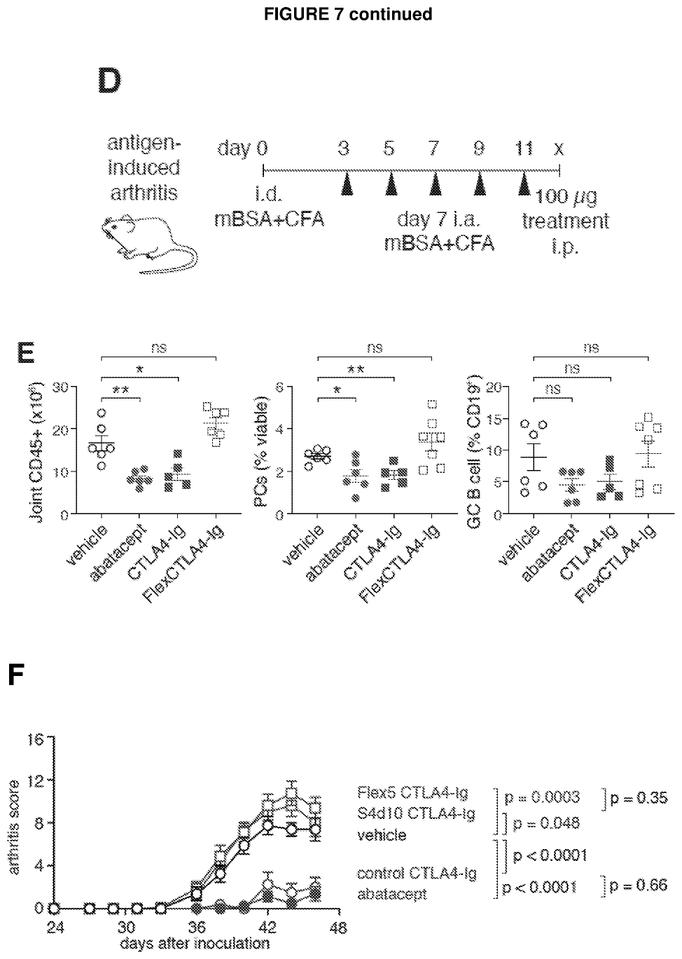

In a mouse model of collagen-induced arthritis, control CTLA4-Ig^{LALA} suppressed disease to a similar extent as clinical grade abatacept verifying Fc-independent effects (FIG. 7A-C). Remarkably, FlexCTLA4-Ig^{LALA} not only failed to suppress arthritis but instead significantly exacerbated disease (FIG. 7B-C).

Similar results were obtained using a FlexCTLA4-Ig^{LALAPG} molecule (Flex5 CTLA4-Ig^{LALAPG} comprising a further mutation in the Fc region at residue 329 (P329G), to disable FcR binding; SEQ ID NO: 57); and using an additional flexible CTLA4-Ig fusion molecule (S4d10 CTLA4-Ig^{LALAPG}, SEQ ID NO: 58). These results are shown in FIG. 7F. The two additional molecules provide further exemplification respectively, of the second aspect of the invention (ie using a CTLA4 sequence derived from the transmembrane form of the protein) and the third aspect of the invention, (ie using a CTLA4 sequence derived from the monomeric soluble form of CTLA4 (SEQ ID NO: 3)). The molecule S4d10 is a C terminally truncated form of the monomeric soluble form of CTLA4 (SEQ ID NO: 3). S4d10, as exemplified in SEQ ID NO: 59, comprises a C terminal truncation to remove a 10 amino acid region that comprises a Cys residue that may result in unwanted disulphide pairing. The resulting CTLA4 sequence (ligand-binding domain plus 12 amino acids downstream, as shown in SEQ ID NO: 59) is then fused to human IgG1 sequence, (SEQ ID NO: 56), forming a homodimeric fusion protein termed S4d10 CTLA4-Ig^{LALAPG} (SEQ ID NO: 58). This sequence was selected for testing because it does not contain Gly/Ser linker sequences, which may render the protein immunogenic.

In another mouse model of T cell-dependent antigen-induced arthritis where abatacept and control CTLA4-Ig^{LALA} effectively reduced inflammation, FlexCTLA4-Ig^{LALA} lacked therapeutic activity (FIG. 7D-E). These data indicate that PD-L1 liberation is essential for in vivo immunosuppression by CTLA4-Ig. Furthermore, they demonstrate a novel in vivo immunostimulatory activity of FlexCTLA4-Ig variants.

### FlexCTLA4-Ig Triggers Steady State Immune Activation In Vivo

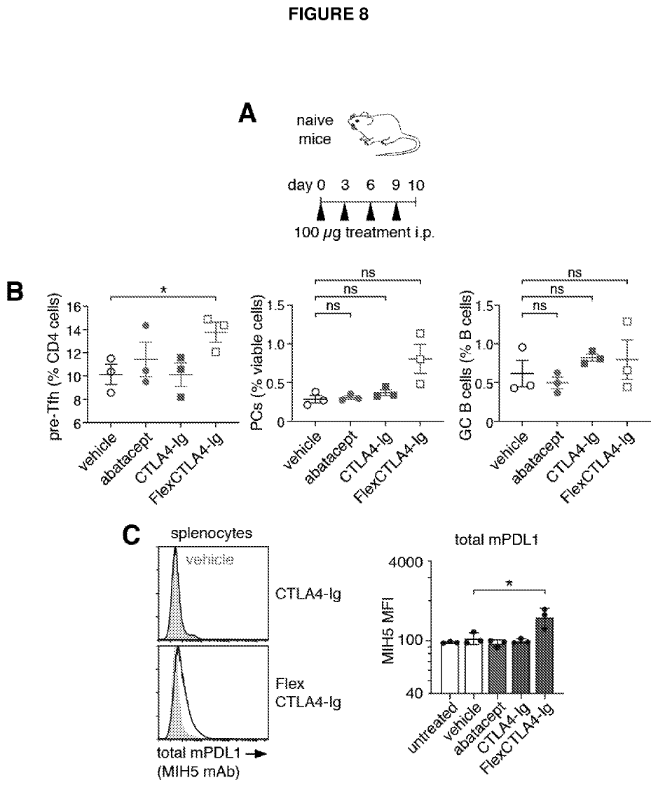

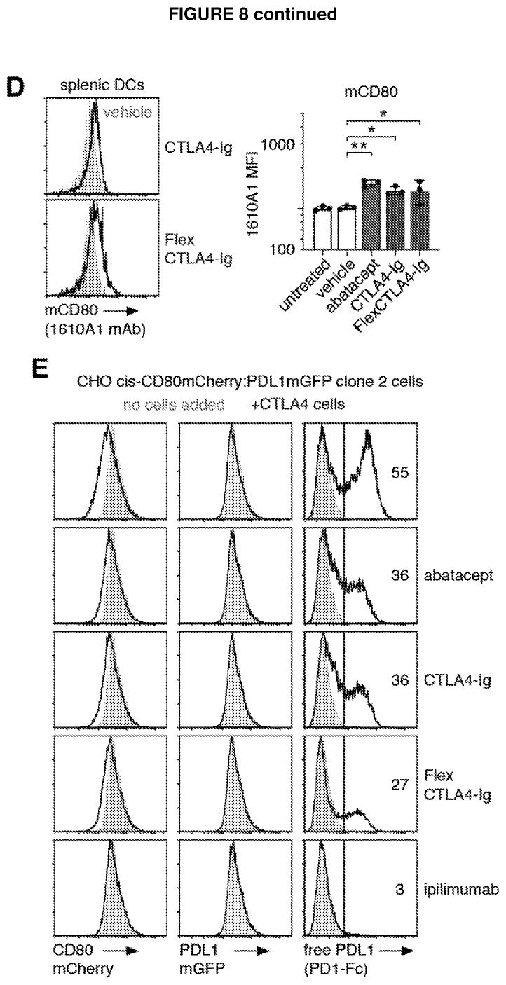

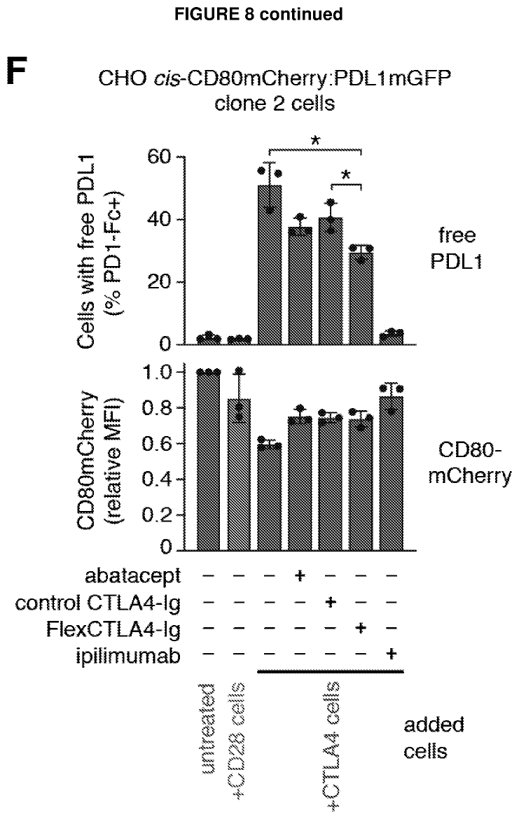

The inventors then examined whether FlexCTLA4-Ig could perturb steady state immune homeostasis (FIG. 8A). Acute treatment of naïve mice with abatacept or control CTLA4-Ig had little effect, however FlexCTLA4-Ig^{LALA} significantly increased the proportion of splenic CXCR5+CD4⁺ T follicular helper (Tfh) cells indicating immune activation (FIG. 8B). Splenocytes from mice treated with FlexCTLA4-Ig^{LALA} also showed widespread induction of surface PD-L1, suggesting a systemic IFN-γ response (FIG. 8C). Notably, all CTLA4-Ig fusion variants increased surface CD80 on splenic DCs in vivo (FIG. 8D). This observation suggests that FlexCTLA4-Ig^{LALA} and control fusions may similarly recognise target APCs in vivo, potentially preventing CD80 transendocytosis by CTLA4-expressing cells including Tregs. Consistent with this, using CHO cis-CD80mCherry:PD-L1mGFP cells, the inventors found that each CTLA4-Ig variant blunted CD80 transendocytosis by CTLA4-expressing cells to a similar extent (FIG. 8E-F). However, when compared to abatacept or control CTLA4-Ig in this assay, FlexCTLA4-Ig significantly reduced PD-L1 liberation from cis-CD80mCherry:PD-L1mGFP complexes (FIG. 8E-F). Together these results suggest that FlexCTLA4-Ig can disrupt immune homeostasis and trigger immune activation, potentially by blocking endogenous CTLA4-induced PD-L1 liberation.

### Y100F and Y100F-Flex Variants Specifically Bind CD80 Proteins but not CD86

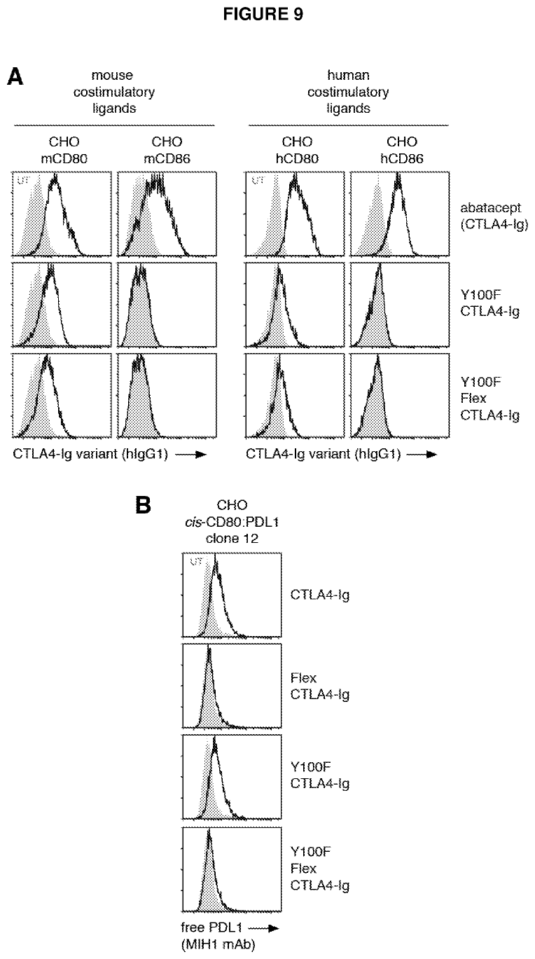

The inventors examined whether the “Flex” version of a known variant of CTLA4, Y100F, which binds to CD80 but not CD86, would trigger liberation of PD-L1. (Y100 corresponds to Y98 in SEQ ID NOs: 1-3 in the present specification). The results in FIG. 9 show binding of abatacept (CTLA4-Ig), Y100F-CTLA4-Ig, and Y100F-Flex-CTLA4-Ig to CHO cells expressing mouse mCD80 or mCD86 (FIG. 9A, left) or human hCD80 or hCD86 (FIG. 9A, right). Across both species the Y100F and Y100F-Flex variants specifically bind CD80 proteins but not CD86 proteins. FIG. 9B shows Free PD-L1 (MIH1 staining) on CHO cis-CD80:PD-L1 cells treated with control CTLA4-Ig, FlexCTLA4-Ig, Y100F-CTLA4-Ig, or Y100F-FlexCTLA4-Ig.

The ‘wild type’ and Y100F versions of CTLA4-Ig both trigger PD-L1 liberation, whereas both versions of FlexCTLA4-Ig fail to liberate PD-L1. These results indicate that binding of CD86 is not required to prevent PD-L1 liberation.

Specific binding of the Y100F and Y100F-Flex variants to CD80 proteins but not CD86 proteins suggests that including the Y100F mutation into CTLA4-Ig variants of the invention may alter their biological functions.

### FlexCTLA4-Ig Induces Anti-Tumor Immunity

Having shown that FlexCTLA4-Ig molecules are immunostimulatory, the inventors next examined whether these molecules could trigger antitumor immunity in the MC38 mouse colon carcinoma model. For these experiments, the inventors utilised Flex5CTLA4-Ig^{LALA} based on its complete lack of mPD-L1 liberation activity.

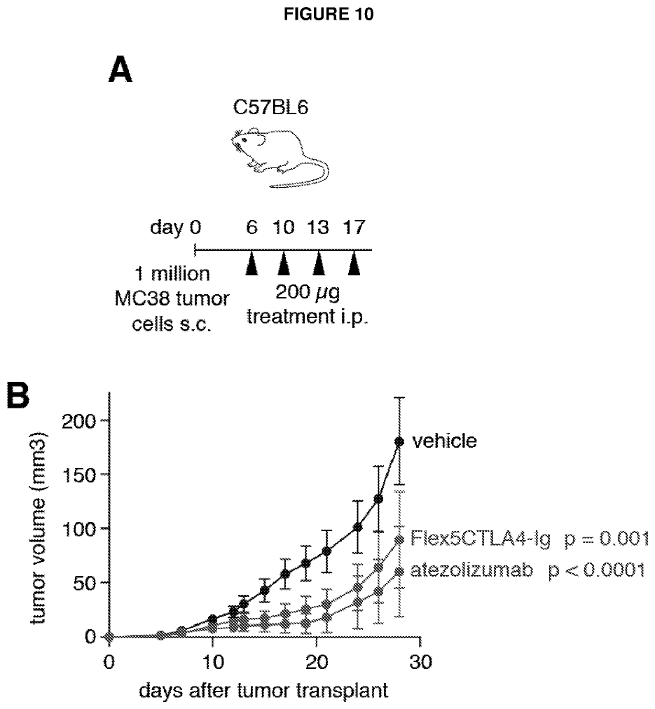

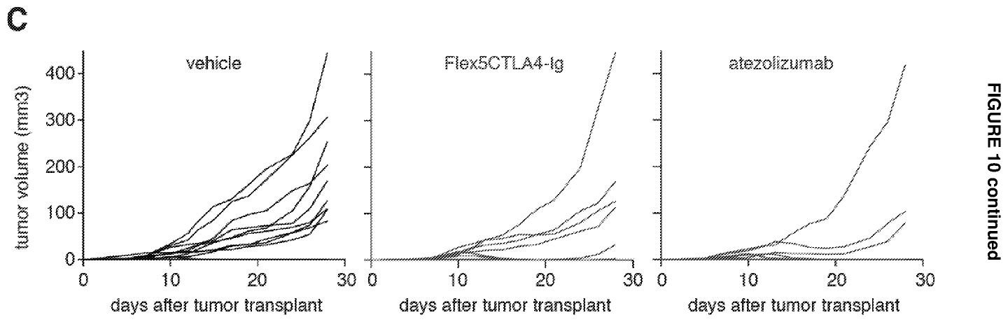

C57BL/6 female mice were injected subcutaneously in one rear flank with 1 million MC38 colon carcinoma cells. Tumors were measured every 2-3 days using electronic calipers and volumes (mm³) calculated as (length×width²×0.5). Treatments were commenced 6 days after tumour cell transplant, with 200 μg of Flex5CTLA4-Ig^{LALA} or atezolizumab administered by intraperitoneal injection twice weekly for two weeks. (FIG. 10A).

Subcutaneously injected MC38 cells rapidly formed tumors, with 9 of 10 vehicle treated mice showing a steady increase in tumor growth (FIG. 10B-C). Only one animal showed spontaneous tumour regression. In contrast, Flex5CTLA4-Ig^{LALA} induced complete tumor regression in 6 of 10 mice within 3 weeks (FIG. 10B-C). Tumor growth suppression by Flex5CTLA4-Ig^{LALA} was comparable to positive control atezolizumab (anti-PDL1), which caused regression in 7 of 10 mice (FIG. 10B-C). Both treatments significantly slowed mean anti-tumour growth but treatment cessation led to tumour re-growth in 1 mouse from the FlexCTLA4-Ig group, providing further evidence of its anti-tumour activity (FIG. 10B-C).

### DISCUSSION

In summary, the inventors show that transmembrane CTLA4 and CTLA4-Ig therapies liberate cell surface PDL1 from cis-bound CD80. This increases PDL1 availability without changing its protein expression, a new mechanism among several regulating its coinhibitory function. PDL1 liberation is triggered by CTLA4-Ig therapies previously thought to simply block costimulation, and may contribute to their potent clinical immunosuppressive effects. PDL1 release also represents a new cell-extrinsic immune-inhibitory mechanism of cell surface CTLA4, alongside CD80/CD86 blockade and transendocytosis.

CTLA4-induced PDL1 liberation does not affect CD86 or PDL2 activity. Furthermore, its effects depend on the relative abundance of CD80 and PDL1 on APCs, which is dynamic in disease states including autoimmunity and cancer. It may also differentially influence the activity of effector T cells and immunosuppressive Tregs. The inventors' data suggest that CTLA4-induced PDL1 liberation is likely to dampen costimulation by dendritic cells, which express abundant cis-CD80:PDL1 complexes and are the primary targets of Treg CTLA4-induced transendocytosis in vivo. CTLA4-induced transendocytosis of CD80 and CD86 can disarm dendritic cells, and simultaneous PDL1 liberation may render them potently tolerogenic.

Cell surface CD80 is normally in a dynamic monomer/dimer equilibrium, but in crystal structure complexes with CTLA4 it is dimeric. Recent mutagenesis studies suggest cis-binding of PDL1 to CD80 occurs at the CD80 homodimerisation interface, implying that CTLA4-induced CD80 dimerisation might displace PDL1. However the inventors' data instead support a model where CTLA4 bridging of two CD80 monomers, not CD80 homodimerisation, displaces PDL1 (FIG. 4I). CTLA4-induced reorganisation of CD80 molecules may subsequently promote interaction of analogous CD80: CTLA4 complexes through CD80 homodimerisation, forming a stable CTLA4:CD80 lattice excluding PDL1 (data not shown). This multi-layered immunosuppressive mechanism may be triggered by therapeutically relevant abatacept concentrations that favour CD80 crosslinking, or at the immune synapse where membrane CTLA4-crosslinked CD80 is enriched.

The inventors have identified a series of flexCTLA4-Ig fusion proteins that block CD80 without liberating PDL1, an effect that may be expected to diminish their immunosuppressive effects relative to abatacept. Surprisingly, rather than simply having a reduced immunosuppressive effect relative, for example, to abatacept, the CTLA4-Ig fusions of the present invention were shown to substantially increase immune activation.

It will be understood that the invention disclosed and defined in this specification extends to all alternative combinations of two or more of the individual features mentioned or evident from the text or drawings. All of these different combinations constitute various alternative aspects of the invention.

1. A fusion protein comprising:
a first portion comprising or consisting of the amino acid sequence of a CTLA4 protein, or functional variant thereof,
a second portion comprising an Fc region of an antibody,
wherein the fusion protein is capable of covalent bonding to form a dimer, preferably a homodimer, and of bivalent ligand binding,
wherein the first portion comprises one or more mutations, insertions, substitutions or deletions for reducing the stability of the CTLA4 homodimerisation interface compared to the homodimerisation interface of wild-type CTLA4 protein,
such that upon bivalent binding of the fusion protein to CD80, PDL1 remains cis-bound to CD80.

2. The fusion protein of claim 1, wherein the covalent bonding is disulphide bonding.

3. The fusion protein of claim 1 or 2, wherein the first portion comprises the amino acid sequence of any of SEQ ID NOs: 1 to 3, or a functional variant thereof having at least 80%, at least 81%, at least 82%, at least 83%, at least 84%, at least 85%, at least 86%, at least 87%, at least 88%, at least 89%, at least 90%, at least 91%, at least 91%, at least 92%, at least 93%, at least 94%, at least 95%, at least 96%, at least 97% at least 98% or at least 99% sequence identity thereto, and one or more mutations, insertions, substitutions or deletions for reducing the stability of the CTLA4 homodimerisation interface compared to the homodimerisation interface of wild-type CTLA4 protein,

4. The fusion protein of any one of claims 1 to 3, wherein the mutation, insertion, substitution or deletion is in a region of the CTLA4 protein involved in formation of hydrophobic interactions between CTLA4 monomers.

5. The fusion protein of any one of claims 1 to 4, wherein the mutation, insertion, substitution or deletion is in a region of the CTLA4 protein corresponding to residues V8 to S13 and/or T110 to C120, or a region equivalent thereto, when numbered according to SEQ ID NO: 2.

6. The fusion protein of any one of claims 1 to 5, wherein the mutation, insertion, substitution or deletion is of or between one or more amino acid residues selected from V8, V9, L10, A11, S12, S13, T110, Q111, 1112, Y113, V114, 1115, D116, P117, E118, and P119, or residues at positions equivalent thereto, in the CTLA4 protein, when numbered according to SEQ ID NO: 2.

7. The fusion protein of any one of claims 1 to 6, wherein the first portion of the fusion protein comprises one or more cysteine residues, capable of forming disulphide bonds to facilitate dimerisation of the fusion protein.

8. The fusion protein of claim 7, wherein the one or more cysteine residues are located in the C-terminal region of the first portion of the fusion protein, preferably within the C-terminal 25 amino acids of the first portion.

9. The fusion protein of claim 8, wherein the one or more cysteine residues is located within 20, 15, 10 or 5 amino acids of the C-terminus of any of SEQ ID NO: 1, 2 or 3, or sequences at least 80%, at least 81%, at least 82%, at least 83%, at least 84%, at least 85%, at least 86%, at least 87%, at least 88%, at least 89%, at least 90%, at least 91%, at least 91%, at least 92%, at least 93%, at least 94%, at least 95%, at least 96%, at least 97% at least 98% or at least 99% sequence identity thereto.

10. The fusion protein of claim 8, wherein the first portion comprises a CTLA4 protein having the amino acid sequence of SEQ ID NO: 2, and or more mutations, insertions, substitutions or deletions for reducing the stability of the CTLA4 homodimerisation interface compared to the homodimerisation interface of wild-type CTLA4 protein, wherein the fusion protein is capable of forming a disulphide bond via the cysteine residue located at residue 120 of SEQ ID NO: 2 or a position equivalent thereto.

11. The fusion protein of any one of claims 1 to 10, wherein the second portion of the fusion protein comprises one or more cysteine residues, capable of forming disulphide bonds to facilitate dimerisation of the fusion protein.

12. The fusion protein of claim 11, wherein the one or more cysteine residues are located in a sequence corresponding to a hinge region of an Fc region of an antibody.

13. The fusion protein of claim 11, wherein the one or more cysteine residues are located in a sequence corresponding to the CH3 domain of an Fc region.

14. A fusion protein comprising:
a first portion comprising an extracellular domain of the transmembrane isoform of CTLA4, or functional variant thereof, wherein the sequence comprises an insertion, substitution or deletion of one or more amino acid residues, located at a position that is within the C-terminal 25 amino acids of the extracellular domain of CTLA4, or position equivalent thereto; and
a second portion comprising an Fc region of an antibody.

15. The fusion protein of claim 14, wherein:
the first portion comprises a variant CTLA4 as compared to the amino acid sequence set forth in SEQ ID NO: 2; and wherein the variant CTLA4 comprises an insertion, substitution or deletion of one or more amino acid residues at a position between residues 100 and 124 of SEQ ID NO: 2, or position equivalent thereto.

16. A fusion protein according to claim 15, wherein the insertion, substitution or deletion results in a flexible domain or destabilisation of the dimerisation interface located at a position of the CTLA4 that is equivalent to the region between residues 100 and 124 of SEQ ID NO: 2.

17. The fusion protein of any one of claims 1 to 16, wherein the insertion, substitution, deletion or flexible domain comprises between about 1 to about 30 amino acid residues.

18. The fusion protein of claim 17, wherein the insertion is at a position of CTLA4 that is equivalent to a position between residues 100 and 101; between residues 101 and 102; between residues 102 and 103; between residues 103 and 104; between residues 104 and 105; between residues 105 and 106; between residues 106 and 107; between residues 107 and 108; between residues 108 and 109; between residues 109 and 110; between residues 110 and 111; between residues 111 and 112; between residues 112 and 113; between residues 113 and 114; between residues 114 and 115; between residues 115 and 116; between residues 116 and 117; between residues 117 and 118; between residues 118 and 119; between residues 119 and 120 of SEQ ID NO: 2; between residues 120 and 121 of SEQ ID NO: 2; between residues 121 and 122 of SEQ ID NO: 2; between residues 122 and 123 of SEQ ID NO: 2, or between residues 123 and 12 of SEQ ID NO: 2.

19. The fusion protein of claim 18, wherein the insertion is at a position of the CTLA4 that is equivalent to between residues 115 and 116 of SEQ ID NO: 2.

20. The fusion protein of any one of claims 1 to 17, wherein the substitution is of a region of CTLA4 that is equivalent to a region comprising any one of residues 110, 111, 112, 113, 114, 115, 116, 117, 118, or residue 119 of SEQ ID NO: 2; or comprises residues 118 and 119 of SEQ ID NO: 2, residues 117 to 119 of SEQ ID NO: 2, residues 116 to 119 of SEQ ID NO: 2, residues 115 to 119 of SEQ ID NO: 2, residues 114 to 119 of SEQ ID NO: 2, residues 113 to 119 of SEQ ID NO: 2, residues 112 to 119 of SEQ ID NO: 2, residues 111 to 119 of SEQ ID NO: 2, residues 110 to 119 of SEQ ID NO: 2, or of regions equivalent thereto.

21. The fusion protein of any one of claims 1 to 17, wherein the deletion is of a region of CTLA4 that is equivalent to a region comprising any one of residues 110, 111, 112, 113, 114, 115, 116, 117, 118, 119, 120, 121, 122, 123 or 124 of SEQ ID NO: 2; or may comprise residues 118 and 119 of SEQ ID NO: 2, residues 117 to 119 of SEQ ID NO: 2, residues 116 to 119 of SEQ ID NO: 2, residues 115 to 119 of SEQ ID NO: 2, residues 114 to 119 of SEQ ID NO: 2, residues 113 to 119 of SEQ ID NO: 2, residues 112 to 119 of SEQ ID NO: 2, residues 111 to 119 of SEQ ID NO: 2, residues 110 to 119 of SEQ ID NO: 2, or of regions equivalent thereto.

22. The fusion protein of any one of claims 1 to 21, wherein the insertion, substitution, or deletion is of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20 or more amino acids.

23. The fusion protein of any one of claims 1 to 22, wherein the insertion or the substitution comprises amino acid residues selected from glycine, serine, alanine, lysine and glutamic acid residues.

24. The fusion protein of any one of claims 14 to 23, wherein the first portion of the fusion protein comprises one or more amino acid residues for enabling covalent bonding to form a dimer, preferably a homodimer.

25. The fusion protein of any one of claims 14 to 23, wherein the second portion of the fusion protein comprises one or more amino acid residues for enabling covalent bonding to form a dimer, preferably a homodimer.

26. The fusion protein of any one of claims 14 to 23, wherein the fusion protein comprises one or more cysteine residues located in the first or second portions of the protein for enabling formation of disulphide bonds to facilitate dimerisation of the fusion protein.

27. The fusion protein of claim 26, wherein the one or more cysteine residues is located in the C-terminal 25 amino acids of the first portion of the fusion protein.

28. The fusion protein of claim 26 or 27 wherein the one or more cysteine residues is located in the N-terminal 20 amino acids of the second portion of the fusion protein, preferably within the hinge region of the Fc region of an antibody.

29. The fusion protein of claim 28, wherein the fusion protein the one or more cysteine residues comprises a cysteine residue located at position 120 of SEQ ID NO: 2, or position equivalent thereto.

30. A fusion protein comprising:
a first portion comprising the sequence of a soluble isoform of CTLA4 or a functional variant or homolog thereof; and
a second portion comprising an Fc region of an antibody.

31. The fusion protein of claim 30, wherein the soluble isoform of CTLA4 comprises an amino acid sequence as set forth in SEQ ID NO: 1 or 3, or a functional variant or homolog thereof.

32. The fusion protein of claim 30 or 31, wherein the fusion protein is capable of covalent bonding to form a dimer, preferably a homodimer, and of bivalent ligand binding.

33. The fusion protein of claim 32, wherein the second portion of the fusion protein comprises one or more amino acid residues for enabling covalent bonding to form a dimer, preferably a homodimer.

34. The fusion protein of any one of claims 30 to 33, wherein the second portion of the fusion protein comprises one or more cysteine residues for enabling formation of disulphide bonds to facilitate dimerisation, preferably homodimerisation of the fusion protein.

35. The fusion protein of claim 34, wherein the one or more cysteine residues is located in the N-terminal 20 amino acids of the second portion of the fusion protein, preferably within the hinge region of the Fc region of an antibody.

36. The fusion protein of any one of claims 30 to 35, wherein the functional variant or homolog of the soluble isoform of CTLA4 comprises or consists of a sequence that is at least 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or at least 99% identical to the amino acid sequence set forth in SEQ ID NO: 1 or 3, wherein the protein retains its ability to bind to a ligand of CTLA4, such as B7-1 (CD80) and/or B7-2 (CD86), optionally wherein the functional variant of the soluble isoform of CTLA4 comprises the amino acid sequence as set forth in SEQ ID NO: 59.

37. The fusion protein of any one of claims 1 to 36, wherein the second portion comprises the sequence of Fc region of an antibody, comprises the amino acid sequence of an Fc region of an immunoglobulin G (Ig), preferably of an IgG1, IgG2, an IgG3 or an IgG4.

38. The fusion protein of claim 37, wherein the second portion comprises the amino acid sequence of an IgG1.

39. The fusion protein of any one of claims 1 to 38, wherein the second portion comprises the amino acid sequence of a variant Fc region.

40. The fusion protein of claim 39, wherein the variant Fc region enhances affinity to the neonatal Fc receptor FcRn and/or extends half-life of the CTLA4-Ig in vivo.

41. The fusion protein of any one of claims 1 to 40, wherein the second portion comprises two heavy chain fragments, more preferably the CH2 and CH3 domains of said heavy chain.

42. The fusion protein of any one of claims 1 to 41, wherein the second portion of the fusion protein comprises the CH2 and CH3 domains of an antibody and an Fc hinge region.

43. The fusion protein of any one of claims 1 to 42, wherein the second portion comprises, consists essentially of or consists of an amino acid sequence set forth in any one of SEQ ID NOs: 17 to 24, or an amino acid sequence having 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence set forth in any one of SEQ ID NOs: 17 to 24.

44. The fusion protein of any one of claims 1 to 29, wherein the second portion comprises, consists essentially of or consists of an amino acid sequence set forth in SEQ ID NO: 24, or an amino acid sequence having 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity to the sequence set forth in SEQ ID NO: 24.

45. The fusion protein of any one of claims 30 to 35, wherein the second portion comprises, consists essentially of or consists of an amino acid sequence set forth in SEQ ID NO: 25, or an amino acid sequence having 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identity the amino acid sequence set forth in SEQ ID NO: 25, 55, 56, 61 or 62.

46. The fusion protein of any one of claims 1 to 45, wherein the first portion is fused at the C-terminus to the second portion.

47. The fusion protein of any one of claims 1 to 46, wherein the first portion of the fusion protein is fused via a linker at the C-terminus to the second portion.

48. The fusion protein of any one of claims 1 to 46, wherein the fusion protein comprises a peptide linker between the first and second portions of the fusion protein.

49. The fusion protein of claim 48, wherein the linker comprises or consists of amino acids.

50. The fusion protein of claim 48 or 49, wherein the linker is selected from flexible linker (such as those comprising repeats of glycine and serine residues), a rigid linker (such as those comprising glutamic acid and lysine residues, flanking alanine repeats) and/or a cleavable linker (such as sequences that are susceptible by protease cleavage).

51. The fusion protein of claim 50, wherein the peptide linker is any one or more repeats of Gly-Gly-Ser (GGS), Gly-Gly-Gly-Ser (GGGS) or Gly-Gly-Gly-Gly-Ser (GGGGS) or variations thereof.

52. The fusion protein of any one of claims 1 to 29, wherein the protein comprises or consists of a sequence as shown in any one of SEQ ID NOs: 7 to 15, 49, 50, 52, 53, 57 or 58.

53. The fusion protein of any one of claims 1 to 29, wherein the protein comprises or consists of the amino acid sequence as set forth in SEQ ID NO: 7.

54. The fusion protein of any one of claims 1 to 29, wherein the protein comprises or consists of the amino acid sequence as set forth in SEQ ID NO: 8.

55. The fusion protein of any one of claims 1 to 29, wherein the protein comprises or consists of the amino acid sequence as set forth in SEQ ID NO: 10.

56. The fusion protein of any one of claims 1 to 29, wherein the protein comprises or consists of the amino acid sequence as set forth in any one of SEQ ID NOs: 7 to 15.

57. The fusion protein of any one of claims 1 to 29, wherein the protein comprises or consists of the amino acid sequence as set forth in SEQ ID NO: 49.

58. The fusion protein of any one of claims 1 to 29, wherein the protein comprises or consists of the amino acid sequence as set forth in SEQ ID NO: 50.

59. The fusion protein of any one of claims 1 to 29, wherein the protein comprises or consists of the amino acid sequence as set forth in SEQ ID NO: 52.

60. The fusion protein of any one of claims 1 to 29, wherein the protein comprises or consists of the amino acid sequence as set forth in SEQ ID NO: 54.

61. The fusion protein of any one of claims 30 to 35, wherein the protein comprises or consists of a sequence as shown in SEQ ID NO: 4.

62. An isolated nucleic acid encoding a fusion protein of any one of claims 1 to 61.

63. A vector comprising a nucleic acid of claim 62, optionally, operably linked to a control sequence.

64. A host cell containing a vector of claim 63 or a nucleic acid of claim 62.

65. A pharmaceutical composition comprising a fusion protein of any one of claims 1 to 61, and a physiologically or pharmaceutically acceptable carrier or diluent.

66. A method of treating cancer or a condition or disorder requiring immunostimulation, in a subject in need thereof, the method comprising administering to said subject, a fusion protein of any one of claims 1 to 61, or pharmaceutical composition of claim 65, thereby treating cancer or a condition or disorder requiring immunostimulation in the subject.

67. Use of a fusion protein of any one of claims 1 to 61, in the manufacture of a medicament for the treatment of cancer or a condition or disorder requiring immunostimulation in a subject.

68. The method of claim 66 or the use of claim 67, wherein the fusion protein comprises the amino acid sequence as set forth in any one of SEQ ID NOs: 7 to 15, 49, 50, 54, 57 or 58.

69. The fusion protein of any one of claims 1 to 61, or pharmaceutical composition of claim 65, for use in the treatment of cancer or a condition or disorder requiring immunostimulation.

70. A dimeric protein formed by covalently bonded, preferably disulphide bonded, monomers of the fusion protein of any one of claims 1 to 61.

71. A dimeric protein formed by covalently bonded, preferably disulphide bonded, monomers of a fusion protein having the amino acid sequence of any one of SEQ ID NOs: 7 to 15, 49, 50, 54, 57 or 58.

72. The dimeric protein of claim 71, wherein the dimeric protein comprises disulphide bonded monomers of proteins having the amino acid sequence of SEQ ID NO: 7.


SEQUENCE LISTING (59 sequences)

SEQ ID NO: 1 - protein, 115 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVI

SEQ ID NO: 2 - protein, 124 aa, synthetic construct [sequence omitted]

SEQ ID NO: 3 - protein, 137 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLCENAPNRARM

SEQ ID NO: 4 - protein, 370 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLCENAPNRARMQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVNLTCLVKGFYPSDIAVEWESNGQPENNYKTTPPVLDSDGSFFLNSTLTVDKSRWQQGNVFSCSVMHEALHNHYTQKSLSLSPGK

SEQ ID NO: 5 - protein, 357 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVNLTCLVKGFYPSDIAVEWESNGQPENNYKTTPPVLDSDGSFFLNSTLTVDKSRWQQGNVFSCSVMHEALHNHYTQKSLSLSPGK

SEQ ID NO: 6 - protein, 357 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 358 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIGDPEPCPDSDQEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 359 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIGSDPEPCPDSDQEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 360 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIGGSDPEPCPDSDQEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 362 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIGGGGSDPEPCPDSDQEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 367 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIGGGGSGGGGSDPEPCPDSDQEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 372 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIGGGGSGGGGSGGGGSDPEPCPDSDQEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 377 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIGGGGSGGGGSGGGGSGGGGSDPEPCPDSDQEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 382 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIGGGGSGGGGSGGGGSGGGGSGGGGSDPEPCPDSDQEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 387 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIGGGGSGGGGSGGGGSGGGGSGGGGSGGGGSDPEPCPDSDQEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 357 aa, synthetic construct [sequence omitted]

SEQ ID NO: 17 - protein, 217 aa, synthetic construct
PAPELLGGPSVFLFPPKPKDTLMISRTPEVTCWVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 217 aa, synthetic construct
PAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 19 - protein, 218 aa, synthetic construct
PAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 20 - protein, 218 aa, synthetic construct
PAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 218 aa, synthetic construct
PAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 217 aa, synthetic construct
PAPELLGGPSVFLFPPKPKDTLMISRTPEVTCWVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 23 - protein, 218 aa, synthetic construct
PAPELLGGSSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 24 - protein, 218 aa, synthetic construct; identical to SEQ ID NO: 21

SEQ ID NO: 25 - protein, 218 aa, synthetic construct
PAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVNLTCLVKGFYPSDIAVEWESNGQPENNYKTTPPVLDSDGSFFLNSTLTVDKSRWQQGNVFSCSVMHEALHNHYTQKSLSLSPGK

SEQ ID NO: 26 - protein, 14 aa, synthetic construct
EPKSCDKTHTCPPC

SEQ ID NO: 27 - protein, 11 aa, synthetic construct
ERKCCVECPPC

SEQ ID NO: 28 - protein, 61 aa, synthetic construct
ELKTPLGDTTHTCPRCPEPKSCDTPPPCPRCPEPKSCDTPPPCPRCPEPKSCDTPPPCPRC

SEQ ID NO: 29 - protein, 11 aa, synthetic construct
ESKYGPPCPSC

SEQ ID NO: 30 - protein, 14 aa, synthetic construct
EPKSSDKTHTSPPS

SEQ ID NO: 31 - protein, 11 aa, synthetic construct
ERKSSVESPPS

SEQ ID NO: 32 - protein, 15 aa, synthetic construct
QEPKSSDKTHTSPPS

SEQ ID NO: 33 - protein, 12 aa, synthetic construct
QERKSSVESPPS

SEQ ID NO: 34 - protein, 15 aa, synthetic construct
QEPKSSDKTHTCPPC

SEQ ID NO: 35 - DNA, 40 bp, synthetic construct
  source 1..40: mol_type = other DNA
ttctctaggcgccggaattcgccaccatggcttgcaattg

SEQ ID NO: 36 - DNA, 44 bp, synthetic construct
  source 1..44: mol_type = other DNA
cggaattgatcccgctcgagctaaaggaagacggtctgttcagc

SEQ ID NO: 37 - DNA, 45 bp, synthetic construct
  source 1..45: mol_type = other DNA
tgtatcgaattcatgaggatatttgctggcattatattcacagcc

SEQ ID NO: 38 - DNA, 43 bp, synthetic construct
  source 1..43: mol_type = other DNA
tctatcctcgagttacgtctcctcgaattgtgtatcatttcgg

SEQ ID NO: 39 - DNA, 32 bp, synthetic construct
  source 1..32: mol_type = other DNA
attacagaattcgccaccatgggccacacacg

SEQ ID NO: 40 - DNA, 34 bp, synthetic construct
  source 1..34: mol_type = other DNA
ctgagactcgagttatacagggcgtacactttcc

SEQ ID NO: 41 - DNA, 45 bp, synthetic construct
  source 1..45: mol_type = other DNA
taagcagaattcgccaccatgaggatatttgctgtctttatattc

SEQ ID NO: 42 - DNA, 36 bp, synthetic construct
  source 1..36: mol_type = other DNA
taagcactcgagttacgtctcctccaaatgtgtatc

SEQ ID NO: 43 - DNA, 36 bp, synthetic construct
  source 1..36: mol_type = other DNA
taagcagaattcgccaccatgcagatcccacaggcg

SEQ ID NO: 44 - DNA, 28 bp, synthetic construct
  source 1..28: mol_type = other DNA
taagcactcgagtcagaggggccaagag

SEQ ID NO: 45 - DNA, 36 bp, synthetic construct
  source 1..36: mol_type = other DNA
taagcagaattcgccaccatgctcaggctgctcttg

SEQ ID NO: 46 - DNA, 30 bp, synthetic construct
  source 1..30: mol_type = other DNA
cctgtactcgagtcaggagcgataggctgc

SEQ ID NO: 47 - DNA, 37 bp, synthetic construct
  source 1..37: mol_type = other DNA
gttctagaattcgccaccatggcttgccttggatttc

SEQ ID NO: 48 - DNA, 45 bp, synthetic construct
  source 1..45: mol_type = other DNA
cctgtactcgagtcaattgatgggaataaaataaggctgaaattg

SEQ ID NO: 49 - protein, 357 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIGGGSCPDSDQEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 357 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKCPDSDQEPKSSDKTHTCPPCPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - DNA, 1077 bp, synthetic construct
  source 1..1077: mol_type = other DNA
gcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgaccaggagcccaagagcagcgacaagacccacacctgccctccttgccctgctccagaggctgctggcggcccttccgtgttcctgtttccccctaagccaaaggacaccctgatgatcagccggaccccagaggtgacatgcgtggtggtggacgtgtcccacgaggaccccgaggtgaagttcaactggtacgtggatggcgtggaggtgcacaatgccaagacaaagcctcgggaggagcagtacaacagcacctatagagtggtgtccgtgctgacagtgctgcaccaggactggctgaacggcaaggagtacaagtgcaaggtgtctaataaggccctgcccgcccctatcgagaagaccatcagcaaggcaaagggacagccaagggagccacaggtgtatacactgccaccctctcgcgacgagctgaccaagaaccaagtgagcctgacatgtctggtgaagggcttctacccctccgacatcgccgtggagtgggagtctaatggccagcctgagaacaattataagaccacacctccagtgctggactccgatggctctttctttctgtactctaagctgacagtggataagagcaggtggcagcagggcaacgtgttcagctgtagcgtgatgcacgaagccctgcacaatcactacactcagaagagcctgtccctgtcacctggaaaatga

SEQ ID NO: 52 - protein, 357 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - DNA, 1080 bp, synthetic construct
  source 1..1080: mol_type = other DNA
gcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattggcgatccagaaccgtgcccagattctgaccaggagcccaagagcagcgacaagacccacacctgccctccttgccctgctccagaggctgctggcggcccttccgtgttcctgtttccccctaagccaaaggacaccctgatgatcagccggaccccagaggtgacatgcgtggtggtggacgtgtcccacgaggaccccgaggtgaagttcaactggtacgtggatggcgtggaggtgcacaatgccaagacaaagcctcgggaggagcagtacaacagcacctatagagtggtgtccgtgctgacagtgctgcaccaggactggctgaacggcaaggagtacaagtgcaaggtgtctaataaggccctgcccgcccctatcgagaagaccatcagcaaggcaaagggacagccaagggagccacaggtgtatacactgccaccctctcgcgacgagctgaccaagaaccaagtgagcctgacatgtctggtgaagggcttctacccctccgacatcgccgtggagtgggagtctaatggccagcctgagaacaattataagaccacacctccagtgctggactccgatggctctttctttctgtactctaagctgacagtggataagagcaggtggcagcagggcaacgtgttcagctgtagcgtgatgcacgaagccctgcacaatcactacactcagaagagcctgtccctgtcacctggaaaatga

SEQ ID NO: 54 - protein, 358 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIGDPEPCPDSDQEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 233 aa, synthetic construct
QEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 233 aa, synthetic construct
QEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 362 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIGGGGSDPEPCPDSDQEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 359 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGLEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 127 aa, synthetic construct
MHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIAKEKKPSYNRGL